(19) United States
(12) Patent Application Publication
(10) Pub. No.: US 2026/0043026 A1
(43) Pub. Date: Feb. 12, 2026

(54) Compositions and Methods for Hydroxyacid Oxidase 1 (HAO1) Gene Editing for Treating Primary Hyperoxaluria Type 1 (PH1)

(71) Applicant: Intellia Therapeutics, Inc., Cambridge, MA (US)

(72) Inventors: Zachary William Dymek, Cambridge, MA (US); Shobu Odate, Arlington, MA (US); Bradley Andrew Murray, Boston, MA (US); Reynald Michael Lescarbeau, Medford, MA (US); Anette Huebner, Somerville, MA (US); Walter Strapps, Dedham, MA (US); Sarah Beth Hesse, Arlington, MA (US)

(73) Assignee: Intellia Therapeutics, Inc., Cambridge, MA (US)

(21) Appl. No.: 19/246,289

(22) Filed: Jun. 23, 2025

Related U.S. Application Data
(63) Continuation of application No. 17/162,377, filed on Jan. 29, 2021, now abandoned.
Application No. 17/162,377 is a continuation of application No. PCT/US2019/044080, filed on Jul. 30, 2019.
(60) Provisional application No. 62/712,904, filed on Jul. 31, 2018.
(60) Provisional application No. 62/738,936, filed on Sep. 28, 2018.
(60) Provisional application No. 62/834,328, filed on Apr. 15, 2019.
(60) Provisional application No. 62/841,734, filed on May 1, 2019.

(51) Int. Cl. C12N 15/113; A61P 13/02; C12N 9/22

(52) U.S. Cl. CPC C12N 15/113; A61P 13/02; C12N 9/22; C12N 2310/122; C12N 2310/313; C12N 2310/321; C12N 2310/322

(57) ABSTRACT

Compositions and methods for editing, e.g., introducing double-stranded breaks, within the HAO1 gene are provided. Compositions and methods for treating subjects having primary hyperoxaluria type 1 (PH1), are provided.

This application is a Continuation of U.S. patent application Ser. No. 17/162,377, filed on Jan. 29, 2021, which is a Continuation of International Application No. PCT/US2019/044080, filed on Jul. 30, 2019, which claims the benefit of U.S. Provisional Patent Application No. 62/712,904, filed Jul. 31, 2018, U.S. Provisional Patent Application No. 62/738,936, filed Sep. 28, 2018, U.S. Provisional Patent Application No. 62/834,328, filed Apr. 15, 2019, and U.S. Provisional Patent Application No. 62/841,734, filed May 1, 2019, the contents of each of which are incorporated herein by reference in their entirety for all purposes.

### REFERENCE TO ELECTRONIC SEQUENCE LISTING

The present application is filed with a Sequence Listing in electronic format. The Sequence Listing is provided as a file entitled “01155-0018-01US-ST26.xml” created on May 7, 2025, which is 304,563 bytes in size. The information in the electronic format of the Sequence Listing is incorporated herein by reference in its entirety.

Primary hyperoxaluria type 1 (PH1) is a genetic disorder characterized by build-up of oxalate. In PH1, mutations are found in the enzyme alanine glyoxylate aminotransferase (AGT or AGT1) that is encoded by the AGXT gene. Normally, AGT converts glyoxylate into glycine in liver peroxisomes. In patients with PH1, mutant AGT is unable to break down glyoxylate, and levels of glyoxylate and its metabolite oxalate increase. Humans cannot oxidize oxalate, and high levels of oxalate in subjects with PH1 cause hyperoxaluria (abnormally high levels of oxalate in the urine).

In PH1, excess oxalate can also combine with calcium to form calcium oxalate in the kidney and other organs. Deposits of calcium oxalate can produce widespread deposition of calcium oxalate (nephrocalcinosis) or formation of kidney and bladder stones (urolithiasis) and lead to kidney damage. Common kidney complications in PH1 include blood in the urine (hematuria), urinary tract infections, kidney damage, and end-stage renal disease (ESRD). Over time, kidneys in patients with PH1 may begin to fail, and levels of oxalate may rise in the blood. Deposition of oxalate in tissues throughout the body, e.g., systemic oxalosis, may occur due to high blood levels of oxalate and can lead to complications in bone, skin, and eye. Patients with PH1 normally have kidney failure at an early age, with renal dialysis or dual kidney/liver organ transplant as the only treatment options.

Hydroxyacid oxidase 1 (HAO1, also known as glycolate oxidase [GOX or GO]) converts glycolate into glyoxylate. It has been proposed that inhibition of HAO1 in individuals with PH1 would block formation of glyoxylate, and excess glycolate would be excreted through the urine. This hypothesis has been tested using knockout animal models, such as those described in Salido E C, et al., PNAS 103(48):18249-18254 (2006). Lumasiran (ALN-GO1), an RNAi therapeutic in clinical trials for the treatment of PH1, targets HAO1 mRNA, and has been shown in early clinical studies to lower urinary oxalate levels.

The idea of treating PH1 by inhibition of HAO1 is further supported by data indicating that a human subject with an abnormal splice variant of HAO1 had asymptomatic glycolic aciduria, whereby there was increased urinary glycolic acid excretion that was not accompanied by apparent kidney pathology (see Frishberg Y et al., J Med Genet 51(8):526-9 (2014). Thus, PH1 could be treated by blocking production of glyoxylate, and thus blocking production of its metabolite oxalate, by inhibition of HAO1 expression.

Approaches using small interfering RNA (siRNA) knockdown or antisense knockdown targeting HAO1 for destruction are also currently being investigated, but while results on short-term suppression of HAO1 expression show encouraging preliminary data (see Liebow et al., J Am Soc Nephrol. 2017 February; 28(2):494-503), a need exists for treatments that can produce long-lasting suppression of HAO1. The present invention provides compositions and methods using the CRISPR/Cas system to knock out the HAO1 gene, thereby reducing the production of HAO1 protein and reducing glyoxylate production in subjects with PH1.

Accordingly, the following embodiments are provided. In some embodiments, the present invention provides compositions and methods using a guide RNA with an RNA-guided DNA binding agent such as the CRISPR/Cas system to substantially reduce or knockout expression of the HAO1 gene, thereby substantially reducing or eliminating the production of GO protein. The substantial reduction or elimination of the production of GO protein through alteration of the HAO1 gene can be a long-term or permanent treatment.

### SUMMARY

The following embodiments are provided.

Embodiment 01 A method of inducing a double-stranded break (DSB) or a single-stranded break (SSB) within the HAO1 gene, comprising delivering a composition to a cell, wherein the composition comprises:
a. a guide RNA comprising
   i. a guide sequence selected from SEQ ID NOs:1-146; or
   ii. at least 17, 18, 19, or 20 contiguous nucleotides of a sequence selected from SEQ ID NOs:1-146; or
   iii. a guide sequence that is at least 99%, 98%, 97%, 96%, 95%, 94%, 93%, 92%, 91%, or 90% identical to a sequence selected from SEQ ID NOs:1-146; or
   iv. a guide sequence comprising any one of SEQ ID Nos: 4, 5, 6, 8, 22, 35, 38, 39, 56, 73, 84, 100, 105, 113, 117, 129, 145; or
   v. a guide sequence comprising any one of SEQ ID No: 8, 22, 35, 39, 73, 84, 100, 105, 113, 145; and optionally
b. an RNA-guided DNA binding agent or a nucleic acid encoding an RNA-guided DNA binding agent.

Embodiment 02 A method of reducing the expression of the HAO1 gene comprising delivering a composition to a cell, wherein the composition comprises:
a. a guide RNA comprising
   i. a guide sequence selected from SEQ ID NOs:1-146; or
   ii. at least 17, 18, 19, or 20 contiguous nucleotides of a sequence selected from SEQ ID NOs:1-146; or
   iii. a guide sequence that is at least 99%, 98%, 97%, 96%, 95%, 94%, 93%, 92%, 91%, or 90% identical to a sequence selected from SEQ ID NOs:1-146; or
   iv. a guide sequence comprising any one of SEQ ID Nos: 4, 5, 6, 8, 22, 35, 38, 39, 56, 73, 84, 100, 105, 113, 117, 129, 145; or
   v. a guide sequence comprising any one of SEQ ID No: 8, 22, 35, 39, 73, 84, 100, 105, 113, 145; and optionally
b. an RNA-guided DNA binding agent or a nucleic acid encoding an RNA-guided DNA binding agent.

Embodiment 03 A method of treating or preventing primary hyperoxaluria type 1 (PH1) comprising administering a composition to a subject in need thereof, wherein the composition comprises:
a. a guide RNA comprising
   i. a guide sequence selected from SEQ ID NOs:1-146; or
   ii. at least 17, 18, 19, or 20 contiguous nucleotides of a sequence selected from SEQ ID NOs:1-146; or
   iii. a guide sequence that is at least 99%, 98%, 97%, 96%, 95%, 94%, 93%, 92%, 91%, or 90% identical to a sequence selected from SEQ ID NOs:1-146; or
   iv. a guide sequence comprising any one of SEQ ID Nos: 4, 5, 6, 8, 22, 35, 38, 39, 56, 73, 84, 100, 105, 113, 117, 129, 145; or
   v. a guide sequence comprising any one of SEQ ID No: 8, 22, 35, 39, 73, 84, 100, 105, 113, 145; and optionally
b. an RNA-guided DNA binding agent or nucleic acid encoding an RNA-guided DNA binding agent, thereby treating or preventing PH1.

Embodiment 04 A method of treating or preventing end stage renal disease (ESRD) caused by PH1 comprising administering a composition to a subject in need thereof, wherein the composition comprises:
a. a guide RNA comprising
   i. a guide sequence selected from SEQ ID NOs:1-146; or
   ii. at least 17, 18, 19, or 20 contiguous nucleotides of a sequence selected from SEQ ID NOs:1-146; or
   iii. a guide sequence that is at least 99%, 98%, 97%, 96%, 95%, 94%, 93%, 92%, 91%, or 90% identical to a sequence selected from SEQ ID NOs:1-146; or
   iv. a guide sequence comprising any one of SEQ ID Nos: 4, 5, 6, 8, 22, 35, 38, 39, 56, 73, 84, 100, 105, 113, 117, 129, 145; or
   v. a guide sequence comprising any one of SEQ ID No: 8, 22, 35, 39, 73, 84, 100, 105, 113, 145; and optionally
b. an RNA-guided DNA binding agent or nucleic acid encoding an RNA-guided DNA binding agent, thereby treating or preventing (ESRD) caused by PH1.

Embodiment 05 A method of treating or preventing any one of calcium oxalate production and deposition, hyperoxaluria, oxalosis, and hematuria comprising administering a composition to a subject in need thereof, wherein the composition comprises:
a. a guide RNA comprising
   i. a guide sequence selected from SEQ ID NOs:1-146; or
   ii. at least 17, 18, 19, or 20 contiguous nucleotides of a sequence selected from SEQ ID NOs:1-146; or
   iii. a guide sequence that is at least 99%, 98%, 97%, 96%, 95%, 94%, 93%, 92%, 91%, or 90% identical to a sequence selected from SEQ ID NOs:1-146; or
   iv. a guide sequence comprising any one of SEQ ID Nos: 4, 5, 6, 8, 22, 35, 38, 39, 56, 73, 84, 100, 105, 113, 117, 129, 145; or
   v. a guide sequence comprising any one of SEQ ID No: 8, 22, 35, 39, 73, 84, 100, 105, 113, 145; and optionally
b. an RNA-guided DNA binding agent or nucleic acid encoding an RNA-guided DNA binding agent, thereby treating or preventing any one of calcium oxalate production and deposition, hyperoxaluria, oxalosis, and hematuria.

Embodiment 06 A method of increasing serum glycolate concentration, comprising administering a composition to a subject in need thereof, wherein the composition comprises:
a. a guide RNA comprising
   i. a guide sequence selected from SEQ ID NOs:1-146; or
   ii. at least 17, 18, 19, or 20 contiguous nucleotides of a sequence selected from SEQ ID NOs:1-146; or
   iii. a guide sequence that is at least 99%, 98%, 97%, 96%, 95%, 94%, 93%, 92%, 91%, or 90% identical to a sequence selected from SEQ ID NOs:1-146; or
   iv. a guide sequence comprising any one of SEQ ID Nos: 4, 5, 6, 8, 22, 35, 38, 39, 56, 73, 84, 100, 105, 113, 117, 129, 145; or
   v. a guide sequence comprising any one of SEQ ID No: 8, 22, 35, 39, 73, 84, 100, 105, 113, 145; and optionally
b. an RNA-guided DNA binding agent or nucleic acid encoding an RNA-guided DNA binding agent, thereby increasing serum glycolate concentration.

Embodiment 07 A method for reducing oxylate in urine in a subject, comprising administering a composition to a subject in need thereof, wherein the composition comprises:
a. a guide RNA comprising
   i. a guide sequence selected from SEQ ID NOs:1-146; or
   ii. at least 17, 18, 19, or 20 contiguous nucleotides of a sequence selected from SEQ ID NOs:1-146; or
   iii. a guide sequence that is at least 99%, 98%, 97%, 96%, 95%, 94%, 93%, 92%, 91%, or 90% identical to a sequence selected from SEQ ID NOs:1-146; or
   iv. a guide sequence comprising any one of SEQ ID Nos: 4, 5, 6, 8, 22, 35, 38, 39, 56, 73, 84, 100, 105, 113, 117, 129, 145; or
   v. a guide sequence comprising any one of SEQ ID No: 8, 22, 35, 39, 73, 84, 100, 105, 113, 145; and optionally
b. an RNA-guided DNA binding agent or nucleic acid encoding an RNA-guided DNA binding agent, thereby reducing oxalate in the urine of a subject.

Embodiment 08 The method of any one of the preceding embodiments, wherein an RNA-guided DNA binding agent or nucleic acid encoding an RNA-guided DNA binding agent is administered.

Embodiment 09 A composition comprising:
a. a guide RNA comprising
   i. a guide sequence selected from SEQ ID NOs:1-146; or
   ii. at least 17, 18, 19, or 20 contiguous nucleotides of a sequence selected from SEQ ID NOs:1-146; or
   iii. a guide sequence that is at least 99%, 98%, 97%, 96%, 95%, 94%, 93%, 92%, 91%, or 90% identical to a sequence selected from SEQ ID NOs:1-146; or
   iv. a guide sequence comprising any one of SEQ ID Nos: 4, 5, 6, 8, 22, 35, 38, 39, 56, 73, 84, 100, 105, 113, 117, 129, 145; or
   v. a guide sequence comprising any one of SEQ ID No: 8, 22, 35, 39, 73, 84, 100, 105, 113, 145; and optionally
b. an RNA-guided DNA binding agent or nucleic acid encoding an RNA-guided DNA binding agent.

Embodiment 10 A composition comprising a short-single guide RNA (short-sgRNA), comprising:
a. a guide sequence comprising:
   i. any one of the guide sequences selected from SEQ ID NOs:1-146; or
   ii. at least 17, 18, 19, or 20 contiguous nucleotides of any one of the guide sequences selected from SEQ ID NOs:1-146; or
   iii. at least 99%, 98%, 97%, 96%, 95%, 94%, 93%, 92%, 91%, or 90% identical to a sequence selected from SEQ ID NOs:1-146; or
   iv. any one of SEQ ID Nos: 4, 5, 6, 8, 22, 35, 38, 39, 56, 73, 84, 100, 105, 113, 117, 129, 145; or
   v. any one of SEQ ID No: 8, 22, 35, 39, 73, 84, 100, 105, 113, 145; and
b. a conserved portion of an sgRNA comprising a hairpin region, wherein the hairpin region lacks at least 5-10 nucleotides and optionally wherein the short-sgRNA comprises one or more of a 5′ end modification and a 3′ end modification.

Embodiment 11 The composition of embodiment 10, comprising the sequence of SEQ ID NO: 202.

Embodiment 12 The composition of embodiment 10 or embodiment 11, comprising a 5′ end modification.

Embodiment 13 The composition of any one of embodiments 10-12, wherein the short-sgRNA comprises a 3′ end modification.

Embodiment 14 The composition of any one of embodiments 10-13, wherein the short-sgRNA comprises a 5′ end modification and a 3′ end modification.

Embodiment 15 The composition of any one of embodiments 10-14, wherein the short-sgRNA further comprises a 3′ tail.

Embodiment 16 The composition of embodiment 15, wherein the 3′ tail comprises 1, 2, 3, 4, 5, 6, 7, 8, 9, or 10 nucleotides.

Embodiment 17 The composition of embodiment 15, wherein the 3′ tail comprises about 1-2, 1-3, 1-4, 1-5, 1-7, 1-10, at least 1-2, at least 1-3, at least 1-4, at least 1-5, at least 1-7, or at least 1-10.

Embodiment 18 The composition of any one of embodiments 10-17, wherein the short-sgRNA does not comprise a 3′ tail.

Embodiment 19 The composition of any one of embodiments 10-18, comprising a modification in the hairpin region.

Embodiment 20 The composition of any one of embodiments 10-19, comprising a 3′ end modification, and a modification in the hairpin region.

Embodiment 21 The composition of any one of embodiments 10-20, comprising a 3′ end modification, a modification in the hairpin region, and a 5′ end modification.

Embodiment 22 The composition of any one of embodiments 10-21, comprising a 5′ end modification, and a modification in the hairpin region.

Embodiment 23 The composition of any one of embodiments 10-22, wherein the hairpin region lacks at least 5 consecutive nucleotides.

Embodiment 24 The composition of any one of embodiments 10-23, wherein the at least 5-10 lacking nucleotides:
a. are within hairpin 1;
b. are within hairpin 1 and the “N” between hairpin 1 and hairpin 2;
c. are within hairpin 1 and the two nucleotides immediately 3′ of hairpin 1;
d. include at least a portion of hairpin 1;
e. are within hairpin 2;
f. include at least a portion of hairpin 2;
g. are within hairpin 1 and hairpin 2;
h. include at least a portion of hairpin 1 and include the “N” between hairpin 1 and hairpin 2;
i. include at least a portion of hairpin 2 and include the “N” between hairpin 1 and hairpin 2;
j. include at least a portion of hairpin 1, include the “N” between hairpin 1 and hairpin 2, and include at least a portion of hairpin 2;
k. are within hairpin 1 or hairpin 2, optionally including the “N” between hairpin 1 and hairpin 2;
l. are consecutive;
m. are consecutive and include the “N” between hairpin 1 and hairpin 2;
n. are consecutive and span at least a portion of hairpin 1 and a portion of hairpin 2;
o. are consecutive and span at least a portion of hairpin 1 and the “N” between hairpin 1 and hairpin 2;
p. are consecutive and span at least a portion of hairpin 1 and two nucleotides immediately 3′ of hairpin 1;
q. consist of 5-10 nucleotides;
r. consist of 6-10 nucleotides;
s. consist of 5-10 consecutive nucleotides;
t. consist of 6-10 consecutive nucleotides; or
u. consist of nucleotides 54-58 of SEQ ID NO:400.

Embodiment 25 The composition of any one of embodiments 10-24, comprising a conserved portion of an sgRNA comprising a nexus region, wherein the nexus region lacks at least one nucleotide.

Embodiment 26 The composition of embodiment 25, wherein the nucleotides lacking in the nexus region comprise any one or more of:
a. at least 2, 3, 4, 5, 6, 7, 8, 9, or 10 nucleotides in the nexus region;
b. at least or exactly 1-2 nucleotides, 1-3 nucleotides, 1-4 nucleotides, 1-5 nucleotides, 1-6 nucleotides, 1-10 nucleotides, or 1-15 nucleotides in the nexus region; and
c. each nucleotide in the nexus region.

Embodiment 27 A composition comprising a modified single guide RNA (sgRNA) comprising
a. a guide sequence comprising:
   i. any one of the guide sequences selected from SEQ ID NOs:1-146; or
   ii. at least 17, 18, 19, or 20 contiguous nucleotides of any one of the guide sequences selected from SEQ ID NOs:1-146; or
   iii. at least 99%, 98%, 97%, 96%, 95%, 94%, 93%, 92%, 91%, or 90% identical to a sequence selected from SEQ ID NOs:1-146; or
   iv. any one of SEQ ID Nos: 4, 5, 6, 8, 22, 35, 38, 39, 56, 73, 84, 100, 105, 113, 117, 129, 145; or
   v. any one of SEQ ID No: 8, 22, 35, 39, 73, 84, 100, 105, 113, 145; and further comprising
b. one or more modifications selected from:
   1. a YA modification at one or more guide region YA sites;
   2. a YA modification at one or more conserved region YA sites;
   3. a YA modification at one or more guide region YA sites and at one or more conserved region YA sites;
   4. i) a YA modification at two or more guide region YA sites;
      ii) a YA modification at one or more of conserved region YA sites 2, 3, 4, and 10; and
      iii) a YA modification at one or more of conserved region YA sites 1 and 8; or
   5. i) a YA modification at one or more guide region YA sites, wherein the guide region YA site is at or after nucleotide 8 from the 5′ end of the 5′ terminus;
      ii) a YA modification at one or more of conserved region YA sites 2, 3, 4, and 10; and optionally;
      iii) a YA modification at one or more of conserved region YA sites 1 and 8; or
   6. i) a YA modification at one or more guide region YA sites, wherein the guide region YA site is within 13 nucleotides of the 3′ terminal nucleotide of the guide region;
      ii) a YA modification at one or more of conserved region YA sites 2, 3, 4, and 10; and
      iii) a YA modification at one or more of conserved region YA sites 1 and 8; or
   7. i) a 5′ end modification and a 3′ end modification;
      ii) a YA modification at one or more of conserved region YA sites 2, 3, 4, and 10; and
      iii) a YA modification at one or more of conserved region YA sites 1 and 8; or
   8. i) a YA modification at a guide region YA site, wherein the modification of the guide region YA site comprises a modification that at least one nucleotide located 5′ of the guide region YA site does not comprise;
      ii) a YA modification at one or more of conserved region YA sites 2, 3, 4, and 10; and
      iii) a YA modification at one or more of conserved region YA sites 1 and 8; or
   9. i) a YA modification at one or more of conserved region YA sites 2, 3, 4, and 10; and
      ii) a YA modification at conserved region YA sites 1 and 8; or
   10. i) a YA modification at one or more guide region YA sites, wherein the YA site is at or after nucleotide 8 from the 5′ terminus;
      ii) a YA modification at one or more of conserved region YA sites 2, 3, 4, and 10; and
      iii) a modification at one or more of H1-1 and H2-1; or
   11. i) a YA modification at one or more of conserved region YA sites 2, 3, 4, and 10; ii) a YA modification at one or more of conserved region YA sites 1, 5, 6, 7, 8, and 9; and iii) a modification at one or more of H1-1 and H2-1; or
   12. i) a modification, such as a YA modification, at one or more nucleotides located at or after nucleotide 6 from the 5′ terminus;
      ii) a YA modification at one or more guide sequence YA sites;
      iii) a modification at one or more of B3, B4, and B5, wherein B6 does not comprise a 2′-OMe modification or comprises a modification other than 2′-OMe;
      iv) a modification at LS10, wherein LS10 comprises a modification other than 2′-fluoro; and/or
      v) a modification at N2, N3, N4, N5, N6, N7, N10, or N11; and wherein at least one of the following is true:
         i. a YA modification at one or more guide region YA sites;
         ii. a YA modification at one or more conserved region YA sites;
         iii. a YA modification at one or more guide region YA sites and at one or more conserved region YA sites;
         iv. at least one of nucleotides 8-11, 13, 14, 17, or 18 from the 5′ end of the 5′ terminus does not comprise a 2′-fluoro modification;
         v. at least one of nucleotides 6-10 from the 5′ end of the 5′ terminus does not comprise a phosphorothioate linkage;
         vi. at least one of B2, B3, B4, or B5 does not comprise a 2′-OMe modification;
         vii. at least one of LS1, LS8, or LS10 does not comprise a 2′-OMe modification;
         viii. at least one of N2, N3, N4, N5, N6, N7, N10, N11, N16, or N17 does not comprise a 2′-OMe modification;
         ix. H1-1 comprises a modification;
         x. H2-1 comprises a modification; or
         xi. at least one of H1-2, H1-3, H1-4, H1-5, H1-6, H1-7, H1-8, H1-9, H1-10, H2-1, H2-2, H2-3, H2-4, H2-5, H2-6, H2-7, H2-8, H2-9, H2-10, H2-11, H2-12, H2-13, H2-14, or H2-15 does not comprise a phosphorothioate linkage.

Embodiment 28 The composition of embodiment 27, comprising SEQ ID NO: 450.

Embodiment 29 The composition of any one of embodiments 9-28, for use in inducing a double-stranded break (DSB) or a single-stranded break within the HAO1 gene in a cell or subject.

Embodiment 30 The composition of any one of embodiments 9-28, for use in reducing the expression of the HAO1 gene in a cell or subject.

Embodiment 31 The composition of any one of embodiments 9-28, for use in treating or preventing PH1 in a subject.

Embodiment 32 The composition of any one of embodiments 9-28, for use in increasing serum and/or plasma glycolate concentration in a subject.

Embodiment 33 The composition of any one of embodiments 9-28, for use in reducing urinary oxalate concentration in a subject.

Embodiment 34 The composition of any one of embodiments 9-28, for use in treating or preventing oxalate production, calcium oxalate deposition in organs, hyperoxaluria, oxalosis, including systemic oxalosis, hematuria, end stage renal disease (ESRD) and/or delaying or ameliorating the need for kidney or liver transplant.

Embodiment 35 The method of any of embodiments 1-8, further comprising:
a. inducing a double-stranded break (DSB) within the HAO1 gene in a cell or subject;
b. reducing the expression of the HAO1 gene in a cell or subject;
c. treating or preventing PH1 in a subject;
d. increasing serum and/or plasma glycolate concentration in a subject;
e. reducing urinary oxalate concentration in a subject;
f. reducing oxalate production;
g. reducing calcium oxalate deposition in organs;
h. reducing hyperoxaluria;
i. treating or preventing oxalosis, including systemic oxalosis;
j. treating or preventing hematuria;
k. preventing end stage renal disease (ESRD); and/or
l. delaying or ameliorating the need for kidney or liver transplant.

Embodiment 36 The method or composition for use of any one of embodiments 1-8 or 29-35, wherein the composition increases serum and/or plasma glycolate levels.

Embodiment 37 The method or composition for use of any one of embodiments 1-8 or 29-35, wherein the composition results in editing of the HAO1 gene.

Embodiment 38 The method or composition for use of embodiment 37, wherein the editing is calculated as a percentage of the population that is edited (percent editing).

Embodiment 39 The method or composition for use of embodiment 38, wherein the percent editing is between 30 and 99% of the population.

Embodiment 40 The method or composition for use of embodiment 38, wherein the percent editing is between 30 and 35%, 35 and 40%, 40 and 45%, 45 and 50%, 50 and 55%, 55 and 60%, 60 and 65%, 65 and 70%, 70 and 75%, 75 and 80%, 80 and 85%, 85 and 90%, 90 and 95%, or 95 and 99% of the population.

Embodiment 41 The method or composition for use of any one of embodiments 1-8 or 29-35, wherein the composition reduces urinary oxalate concentration.

Embodiment 42 The method or composition for use of embodiment 41, wherein a reduction in urinary oxalate results in decreased kidney stones and/or calcium oxalate deposition in the kidney, liver, bladder, heart, skin or eye.

Embodiment 43 The method, composition for use, or composition of any one of the preceding embodiments, wherein the guide sequence is selected from
a. SEQ ID NOs:1-146;
b. SEQ ID Nos: 4, 5, 6, 8, 22, 35, 38, 39, 56, 73, 84, 100, 105, 113, 117, 129, 145; and
c. SEQ ID No: 8, 22, 35, 39, 73, 84, 100, 105, 113, 145.

Embodiment 44 The method, composition for use, or composition of any one of the preceding embodiments, wherein the composition comprises a sgRNA comprising
a. any one of SEQ ID NOs: 151-168; or
b. any one of SEQ ID NOs: 251-268; or
c. a guide sequence selected from SEQ ID Nos: 4, 5, 6, 8, 22, 35, 38, 39, 56, 73, 84, 100, 105, 113, 117, 129, 145; or
d. a guide sequence selected from SEQ ID Nos: 8, 22, 35, 39, 73, 84, 100, 105, 113, 145.

Embodiment 45 The method, composition for use, or composition of any one of the preceding embodiments, wherein the target sequence is in exon 1, 3, 4, 5, 6 or 8 of the human HAO1 gene.

Embodiment 46 The method, composition for use, or composition of embodiment 45, wherein the target sequence is in exon 1 of the human HAO1 gene.

Embodiment 47 The method, composition for use, or composition of embodiment 45, wherein the target sequence is in exon 3 of the human HAO1 gene.

Embodiment 48 The method, composition for use, or composition of embodiment 45, wherein the target sequence is in exon 4 of the human HAO1 gene.

Embodiment 49 The method, composition for use, or composition of embodiment 45, wherein the target sequence is in exon 6 of the human HAO1 gene.

Embodiment 50 The method, composition for use, or composition of embodiment 45, wherein the target sequence is in exon 8 of the human HAO1 gene.

Embodiment 51 The method, composition for use, or composition of any one of embodiments 1-50, wherein the guide sequence is complementary to a target sequence in the positive strand of HAO1.

Embodiment 52 The method, composition for use, or composition of any one of embodiments 1-50, wherein the guide sequence is complementary to a target sequence in the negative strand of HAO1.

Embodiment 53 The method, composition for use, or composition of any one of embodiments 1-50, wherein the first guide sequence is complementary to a first target sequence in the positive strand of the HAO1 gene, and wherein the composition further comprises a second guide sequence that is complementary to a second target sequence in the negative strand of the HAO1 gene.

Embodiment 54 The method, composition for use, or composition of any one of the preceding embodiments, wherein the guide RNA comprises a guide sequence selected from any one of SEQ ID Nos 1-146 and further comprises a nucleotide sequence of SEQ ID NO: 200, wherein the nucleotides of SEQ ID NO: 200 follow the guide sequence at its 3′ end.

Embodiment 55 The method, composition for use, or composition of any one of the preceding embodiments, wherein the guide RNA comprises a guide sequence selected from any one of SEQ ID Nos 1-146 and further comprises a nucleotide sequence of SEQ ID NO: 201, SEQ ID NO: 202, SEQ ID NO: 203, or any one of SEQ ID Nos: 400-450, wherein the nucleotides of SEQ ID NO: 201, SEQ ID NO: 202, SEQ ID NO: 203, or any one of SEQ ID Nos: 400-450 follow the guide sequence at its 3′ end.

Embodiment 56 The method, composition for use, or composition of any one of the preceding embodiments, wherein the guide RNA is a single guide (sgRNA).

Embodiment 57 The method, composition for use, or composition of embodiment 56, wherein the sgRNA comprises a guide sequence comprising any one of SEQ ID Nos: 4, 5, 6, 8, 22, 35, 38, 39, 56, 73, 84, 100, 105, 113, 117, 129, or 145.

Embodiment 58 The method, composition for use, or composition of embodiment 56, wherein the sgRNA comprises any one of SEQ ID Nos: 151-168 or 251-268.

Embodiment 59 The method, composition for use, or composition of any one of the preceding embodiments, wherein the guide RNA is modified according to the pattern of SEQ ID NO: 300, wherein the N's are collectively any one of the guide sequences of Table 1 (SEQ ID Nos 1-146).

Embodiment 60 The method, composition for use, or composition of embodiment 59, wherein each N in SEQ ID NO: 300 is any natural or non-natural nucleotide, wherein the N's form the guide sequence, and the guide sequence targets Cas9 to the HAO1 gene.

Embodiment 61 The method, composition for use, or composition of any one of the preceding embodiments, wherein the sgRNA comprises any one of the guide sequences of SEQ ID NOs:1-146 and the nucleotides of SEQ ID NO: 201, SEQ ID NO: 202, or SEQ ID NO: 203, wherein the nucleotides of SEQ ID NO: 201, SEQ ID NO: 202, or SEQ ID NO: 203 follow the guide sequence at its 3′ end.

Embodiment 62 The method, composition for use, or composition of any one of the preceding embodiments, wherein the sgRNA comprises a guide sequence that is at least 99%, 98%, 97%, 96%, 95%, 94%, 93%, 92%, 91%, or 90% identical to a sequence selected from SEQ ID Nos: 1-146.

Embodiment 63 The method, composition for use, or composition of embodiment 62, wherein the sgRNA comprises a sequence selected from SEQ ID Nos: 8, 22, 35, 39, 73, 84, 100, 105, 113, 145, 151-168, and 251-268.

Embodiment 64 The method, composition for use, or composition of any one of the preceding embodiments, wherein the guide RNA comprises at least one modification.

Embodiment 65 The method, composition for use, or composition of embodiment 64, wherein the at least one modification includes a 2′-O-methyl (2′-O-Me) modified nucleotide.

Embodiment 66 The method, composition for use, or composition of any one of embodiments 63-65, comprising a phosphorothioate (PS) bond between nucleotides.

Embodiment 67 The method, composition for use, or composition of any one of embodiments 63-66, comprising a 2′-fluoro (2′-F) modified nucleotide.

Embodiment 68 The method, composition for use, or composition of any one of embodiments 63-67, comprising a modification at one or more of the first five nucleotides at the 5′ end of the guide RNA.

Embodiment 69 The method, composition for use, or composition of any one of embodiments 63-68, comprising a modification at one or more of the last five nucleotides at the 3′ end of the guide RNA.

Embodiment 70 The method, composition for use, or composition of any one of embodiments 63-69, comprising a PS bond between the first four nucleotides of the guide RNA.

Embodiment 71 The method, composition for use, or composition of any one of embodiments 63-70, comprising a PS bond between the last four nucleotides of the guide RNA.

Embodiment 72 The method, composition for use, or composition of any one of embodiments 63-71, comprising a 2′-O-Me modified nucleotide at the first three nucleotides at the 5′ end of the guide RNA.

Embodiment 73 The method, composition for use, or composition of any one of embodiments 63-72, comprising a 2′-O-Me modified nucleotide at the last three nucleotides at the 3′ end of the guide RNA.

Embodiment 74 The method, composition for use, or composition of any one of embodiments 63-73, wherein the guide RNA comprises the modified nucleotides of SEQ ID NO: 300.

Embodiment 75 The method, composition for use, or composition of any one of embodiments 1-74, wherein the composition further comprises a pharmaceutically acceptable excipient.

Embodiment 76 The method, composition for use, or composition of any one of embodiments 1-75, wherein the guide RNA is associated with a lipid nanoparticle (LNP).

Embodiment 77 The method, composition for use, or composition of embodiment 76, wherein the LNP comprises a cationic lipid.

Embodiment 78 The method, composition for use, or composition of embodiment 77, wherein the cationic lipid is (9Z,12Z)-3-((4,4-bis(octyloxy)butanoyl)oxy)-2-((((3-(diethylamino)propoxy)carbonyl)oxy)methyl)propyl octadeca-9,12-dienoate, also called 3-((4,4-bis(octyloxy)butanoyl)oxy)-2-((((3-(diethylamino)propoxy)carbonyl)oxy)methyl)propyl (9Z,12Z)-octadeca-9,12-dienoate.

Embodiment 79 The method, composition for use, or composition of any one of embodiments 76-78, wherein the LNP comprises a neutral lipid.

Embodiment 80 The method, composition for use, or composition of embodiment 79, wherein the neutral lipid is DSPC.

Embodiment 81 The method, composition for use, or composition of any one of embodiments 76-80, wherein the LNP comprises a helper lipid.

Embodiment 82 The method, composition for use, or composition of embodiment 81, wherein the helper lipid is cholesterol.

Embodiment 83 The method, composition for use, or composition of any one of embodiments 76-82, wherein the LNP comprises a stealth lipid.

Embodiment 84 The method, composition for use, or composition of embodiment 83, wherein the stealth lipid is PEG2k-DMG.

Embodiment 85 The method, composition for use, or composition of any one of the preceding embodiments, wherein the composition further comprises an RNA-guided DNA binding agent.

Embodiment 86 The method, composition for use, or composition of any one of the preceding embodiments, wherein the composition further comprises an mRNA that encodes an RNA-guided DNA binding agent.

Embodiment 87 The method, composition for use, or composition of embodiment 85 or 86, wherein the RNA-guided DNA binding agent is Cas9.

Embodiment 88 The method, composition for use, or composition of any one of the preceding embodiments, wherein the composition is a pharmaceutical formulation and further comprises a pharmaceutically acceptable carrier.

Embodiment 89 The method, composition for use, or composition of any one of embodiments 1-88, wherein the sequence selected from SEQ ID NOs:1-146 is SEQ ID NO: 1.

Embodiment 90 The method, composition for use, or composition of any one of embodiments 1-88, wherein the sequence selected from SEQ ID NOs:1-146 is SEQ ID NO: 2.

Embodiment 91 The method, composition for use, or composition of any one of embodiments 1-88, wherein the sequence selected from SEQ ID NOs:1-146 is SEQ ID NO: 3.

Embodiment 92 The method, composition for use, or composition of any one of embodiments 1-88, wherein the sequence selected from SEQ ID NOs:1-146 is SEQ ID NO: 4.

Embodiment 93 The method, composition for use, or composition of any one of embodiments 1-88, wherein the sequence selected from SEQ ID NOs:1-146 is SEQ ID NO: 5.

Embodiment 94 The method, composition for use, or composition of any one of embodiments 1-88, wherein the sequence selected from SEQ ID NOs:1-146 is SEQ ID NO: 6.

Embodiment 95 The method, composition for use, or composition of any one of embodiments 1-88, wherein the sequence selected from SEQ ID NOs:1-146 is SEQ ID NO: 7.

Embodiment 96 The method, composition for use, or composition of any one of embodiments 1-88, wherein the sequence selected from SEQ ID NOs:1-146 is SEQ ID NO: 8.

Embodiment 97 The method, composition for use, or composition of any one of embodiments 1-88, wherein the sequence selected from SEQ ID NOs:1-146 is SEQ ID NO: 9.

Embodiment 98 The method, composition for use, or composition of any one of embodiments 1-88, wherein the sequence selected from SEQ ID NOs:1-146 is SEQ ID NO: 10.

Embodiment 99 The method, composition for use, or composition of any one of embodiments 1-88, wherein the sequence selected from SEQ ID NOs:1-146 is SEQ ID NO: 11.

Embodiment 100 The method, composition for use, or composition of any one of embodiments 1-88, wherein the sequence selected from SEQ ID NOs:1-146 is SEQ ID NO: 12.

Embodiment 101 The method, composition for use, or composition of any one of embodiments 1-88, wherein the sequence selected from SEQ ID NOs:1-146 is SEQ ID NO: 13.

Embodiment 102 The method, composition for use, or composition of any one of embodiments 1-88, wherein the sequence selected from SEQ ID NOs:1-146 is SEQ ID NO: 14.

Embodiment 103 The method, composition for use, or composition of any one of embodiments 1-88, wherein the sequence selected from SEQ ID NOs:1-146 is SEQ ID NO: 15.

Embodiment 104 The method, composition for use, or composition of any one of embodiments 1-88, wherein the sequence selected from SEQ ID NOs:1-146 is SEQ ID NO: 16.

Embodiment 105 The method, composition for use, or composition of any one of embodiments 1-88, wherein the sequence selected from SEQ ID NOs:1-146 is SEQ ID NO: 17.

Embodiment 106 The method, composition for use, or composition of any one of embodiments 1-88, wherein the sequence selected from SEQ ID NOs:1-146 is SEQ ID NO: 18.

Embodiment 107 The method, composition for use, or composition of any one of embodiments 1-88, wherein the sequence selected from SEQ ID NOs:1-146 is SEQ ID NO: 19.

Embodiment 108 The method, composition for use, or composition of any one of embodiments 1-88, wherein the sequence selected from SEQ ID NOs:1-146 is SEQ ID NO: 20.

Embodiment 109 The method, composition for use, or composition of any one of embodiments 1-88, wherein the sequence selected from SEQ ID NOs:1-146 is SEQ ID NO: 21.

Embodiment 110 The method, composition for use, or composition of any one of embodiments 1-88, wherein the sequence selected from SEQ ID NOs:1-146 is SEQ ID NO: 22.

Embodiment 111 The method, composition for use, or composition of any one of embodiments 1-88, wherein the sequence selected from SEQ ID NOs:1-146 is SEQ ID NO: 23.

Embodiment 112 The method, composition for use, or composition of any one of embodiments 1-88, wherein the sequence selected from SEQ ID NOs:1-146 is SEQ ID NO: 24.

Embodiment 113 The method, composition for use, or composition of any one of embodiments 1-88, wherein the sequence selected from SEQ ID NOs:1-146 is SEQ ID NO: 25.

Embodiment 114 The method, composition for use, or composition of any one of embodiments 1-88, wherein the sequence selected from SEQ ID NOs:1-146 is SEQ ID NO: 26.

Embodiment 115 The method, composition for use, or composition of any one of embodiments 1-88, wherein the sequence selected from SEQ ID NOs:1-146 is SEQ ID NO: 27.

Embodiment 116 The method, composition for use, or composition of any one of embodiments 1-88, wherein the sequence selected from SEQ ID NOs:1-146 is SEQ ID NO: 28.

Embodiment 117 The method, composition for use, or composition of any one of embodiments 1-88, wherein the sequence selected from SEQ ID NOs:1-146 is SEQ ID NO: 29.

Embodiment 118 The method, composition for use, or composition of any one of embodiments 1-88, wherein the sequence selected from SEQ ID NOs:1-146 is SEQ ID NO: 30.

Embodiment 119 The method, composition for use, or composition of any one of embodiments 1-88, wherein the sequence selected from SEQ ID NOs:1-146 is SEQ ID NO: 31.

Embodiment 120 The method, composition for use, or composition of any one of embodiments 1-88, wherein the sequence selected from SEQ ID NOs:1-146 is SEQ ID NO: 32.

Embodiment 121 The method, composition for use, or composition of any one of embodiments 1-88, wherein the sequence selected from SEQ ID NOs:1-146 is SEQ ID NO: 33.

Embodiment 122 The method, composition for use, or composition of any one of embodiments 1-88, wherein the sequence selected from SEQ ID NOs:1-146 is SEQ ID NO: 34.

Embodiment 123 The method, composition for use, or composition of any one of embodiments 1-88, wherein the sequence selected from SEQ ID NOs:1-146 is SEQ ID NO: 35.

Embodiment 124 The method, composition for use, or composition of any one of embodiments 1-88, wherein the sequence selected from SEQ ID NOs:1-146 is SEQ ID NO: 36.

Embodiment 125 The method, composition for use, or composition of any one of embodiments 1-88, wherein the sequence selected from SEQ ID NOs:1-146 is SEQ ID NO: 37.

Embodiment 126 The method, composition for use, or composition of any one of embodiments 1-88, wherein the sequence selected from SEQ ID NOs:1-146 is SEQ ID NO: 38.

Embodiment 127 The method, composition for use, or composition of any one of embodiments 1-88, wherein the sequence selected from SEQ ID NOs:1-146 is SEQ ID NO: 39.

Embodiment 128 The method, composition for use, or composition of any one of embodiments 1-88, wherein the sequence selected from SEQ ID NOs:1-146 is SEQ ID NO: 40.

Embodiment 129 The method, composition for use, or composition of any one of embodiments 1-88, wherein the sequence selected from SEQ ID NOs:1-146 is SEQ ID NO: 41.

Embodiment 130 The method, composition for use, or composition of any one of embodiments 1-88, wherein the sequence selected from SEQ ID NOs:1-146 is SEQ ID NO: 42.

Embodiment 131 The method, composition for use, or composition of any one of embodiments 1-88, wherein the sequence selected from SEQ ID NOs:1-146 is SEQ ID NO: 43.

Embodiment 132 The method, composition for use, or composition of any one of embodiments 1-88, wherein the sequence selected from SEQ ID NOs:1-146 is SEQ ID NO: 44.

Embodiment 133 The method, composition for use, or composition of any one of embodiments 1-88, wherein the sequence selected from SEQ ID NOs:1-146 is SEQ ID NO: 45.

Embodiment 134 The method, composition for use, or composition of any one of embodiments 1-88, wherein the sequence selected from SEQ ID NOs:1-146 is SEQ ID NO: 46.

Embodiment 135 The method, composition for use, or composition of any one of embodiments 1-88, wherein the sequence selected from SEQ ID NOs:1-146 is SEQ ID NO: 47.

Embodiment 136 The method, composition for use, or composition of any one of embodiments 1-88, wherein the sequence selected from SEQ ID NOs:1-146 is SEQ ID NO: 48.

Embodiment 137 The method, composition for use, or composition of any one of embodiments 1-88, wherein the sequence selected from SEQ ID NOs:1-146 is SEQ ID NO: 49.

Embodiment 138 The method, composition for use, or composition of any one of embodiments 1-88, wherein the sequence selected from SEQ ID NOs:1-146 is SEQ ID NO: 50.

Embodiment 139 The method, composition for use, or composition of any one of embodiments 1-88, wherein the sequence selected from SEQ ID NOs:1-146 is SEQ ID NO: 51.

Embodiment 140 The method, composition for use, or composition of any one of embodiments 1-88, wherein the sequence selected from SEQ ID NOs:1-146 is SEQ ID NO: 52.

Embodiment 141 The method, composition for use, or composition of any one of embodiments 1-88, wherein the sequence selected from SEQ ID NOs:1-146 is SEQ ID NO: 53.

Embodiment 142 The method, composition for use, or composition of any one of embodiments 1-88, wherein the sequence selected from SEQ ID NOs:1-146 is SEQ ID NO: 54.

Embodiment 143 The method, composition for use, or composition of any one of embodiments 1-88, wherein the sequence selected from SEQ ID NOs:1-146 is SEQ ID NO: 55.

Embodiment 144 The method, composition for use, or composition of any one of embodiments 1-88, wherein the sequence selected from SEQ ID NOs:1-146 is SEQ ID NO: 56.

Embodiment 145 The method, composition for use, or composition of any one of embodiments 1-88, wherein the sequence selected from SEQ ID NOs:1-146 is SEQ ID NO: 57.

Embodiment 146 The method, composition for use, or composition of any one of embodiments 1-88, wherein the sequence selected from SEQ ID NOs:1-146 is SEQ ID NO: 58.

Embodiment 147 The method, composition for use, or composition of any one of embodiments 1-88, wherein the sequence selected from SEQ ID NOs:1-146 is SEQ ID NO: 59.

Embodiment 148 The method, composition for use, or composition of any one of embodiments 1-88, wherein the sequence selected from SEQ ID NOs:1-146 is SEQ ID NO: 60.

Embodiment 149 The method, composition for use, or composition of any one of embodiments 1-88, wherein the sequence selected from SEQ ID NOs:1-146 is SEQ ID NO: 61.

Embodiment 150 The method, composition for use, or composition of any one of embodiments 1-88, wherein the sequence selected from SEQ ID NOs:1-146 is SEQ ID NO: 62.

Embodiment 151 The method, composition for use, or composition of any one of embodiments 1-88, wherein the sequence selected from SEQ ID NOs:1-146 is SEQ ID NO: 63.

Embodiment 152 The method, composition for use, or composition of any one of embodiments 1-88, wherein the sequence selected from SEQ ID NOs:1-146 is SEQ ID NO: 64.

Embodiment 153 The method, composition for use, or composition of any one of embodiments 1-88, wherein the sequence selected from SEQ ID NOs:1-146 is SEQ ID NO: 65.

Embodiment 154 The method, composition for use, or composition of any one of embodiments 1-88, wherein the sequence selected from SEQ ID NOs:1-146 is SEQ ID NO: 66.

Embodiment 155 The method, composition for use, or composition of any one of embodiments 1-88, wherein the sequence selected from SEQ ID NOs:1-146 is SEQ ID NO: 67.

Embodiment 156 The method, composition for use, or composition of any one of embodiments 1-88, wherein the sequence selected from SEQ ID NOs:1-146 is SEQ ID NO: 68.

Embodiment 157 The method, composition for use, or composition of any one of embodiments 1-88, wherein the sequence selected from SEQ ID NOs:1-146 is SEQ ID NO: 69.

Embodiment 158 The method, composition for use, or composition of any one of embodiments 1-88, wherein the sequence selected from SEQ ID NOs:1-146 is SEQ ID NO: 70.

Embodiment 159 The method, composition for use, or composition of any one of embodiments 1-88, wherein the sequence selected from SEQ ID NOs:1-146 is SEQ ID NO: 71.

Embodiment 160 The method, composition for use, or composition of any one of embodiments 1-88, wherein the sequence selected from SEQ ID NOs:1-146 is SEQ ID NO: 72.

Embodiment 161 The method, composition for use, or composition of any one of embodiments 1-88, wherein the sequence selected from SEQ ID NOs:1-146 is SEQ ID NO: 73.

Embodiment 162 The method, composition for use, or composition of any one of embodiments 1-88, wherein the sequence selected from SEQ ID NOs:1-146 is SEQ ID NO: 74.

Embodiment 163 The method, composition for use, or composition of any one of embodiments 1-88, wherein the sequence selected from SEQ ID NOs:1-146 is SEQ ID NO: 75.

Embodiment 164 The method, composition for use, or composition of any one of embodiments 1-88, wherein the sequence selected from SEQ ID NOs:1-146 is SEQ ID NO: 76.

Embodiment 165 The method, composition for use, or composition of any one of embodiments 1-88, wherein the sequence selected from SEQ ID NOs:1-146 is SEQ ID NO: 77.

Embodiment 166 The method, composition for use, or composition of any one of embodiments 1-88, wherein the sequence selected from SEQ ID NOs:1-146 is SEQ ID NO: 78.

Embodiment 167 The method, composition for use, or composition of any one of embodiments 1-88, wherein the sequence selected from SEQ ID NOs:1-146 is SEQ ID NO: 79.

Embodiment 168 The method, composition for use, or composition of any one of embodiments 1-88, wherein the sequence selected from SEQ ID NOs:1-146 is SEQ ID NO: 80.

Embodiment 169 The method, composition for use, or composition of any one of embodiments 1-88, wherein the sequence selected from SEQ ID NOs:1-146 is SEQ ID NO: 81.

Embodiment 170 The method, composition for use, or composition of any one of embodiments 1-88, wherein the sequence selected from SEQ ID NOs:1-146 is SEQ ID NO: 82.

Embodiment 171 The method, composition for use, or composition of any one of embodiments 1-88, wherein the sequence selected from SEQ ID NOs:1-146 is SEQ ID NO: 83.

Embodiment 172 The method, composition for use, or composition of any one of embodiments 1-88, wherein the sequence selected from SEQ ID NOs:1-146 is SEQ ID NO: 84.

Embodiment 173 The method, composition for use, or composition of any one of embodiments 1-88, wherein the sequence selected from SEQ ID NOs:1-146 is SEQ ID NO: 85.

Embodiment 174 The method, composition for use, or composition of any one of embodiments 1-88, wherein the sequence selected from SEQ ID NOs:1-146 is SEQ ID NO: 86.

Embodiment 175 The method, composition for use, or composition of any one of embodiments 1-88, wherein the sequence selected from SEQ ID NOs:1-146 is SEQ ID NO: 87.

Embodiment 176 The method, composition for use, or composition of any one of embodiments 1-88, wherein the sequence selected from SEQ ID NOs:1-146 is SEQ ID NO: 88.

Embodiment 177 The method, composition for use, or composition of any one of embodiments 1-88, wherein the sequence selected from SEQ ID NOs:1-146 is SEQ ID NO: 89.

Embodiment 178 The method, composition for use, or composition of any one of embodiments 1-88, wherein the sequence selected from SEQ ID NOs:1-146 is SEQ ID NO: 90.

Embodiment 179 The method, composition for use, or composition of any one of embodiments 1-88, wherein the sequence selected from SEQ ID NOs:1-146 is SEQ ID NO: 91.

Embodiment 180 The method, composition for use, or composition of any one of embodiments 1-88, wherein the sequence selected from SEQ ID NOs:1-146 is SEQ ID NO: 92.

Embodiment 181 The method, composition for use, or composition of any one of embodiments 1-88, wherein the sequence selected from SEQ ID NOs:1-146 is SEQ ID NO: 93.

Embodiment 182 The method, composition for use, or composition of any one of embodiments 1-88, wherein the sequence selected from SEQ ID NOs:1-146 is SEQ ID NO: 94.

Embodiment 183 The method, composition for use, or composition of any one of embodiments 1-88, wherein the sequence selected from SEQ ID NOs:1-146 is SEQ ID NO: 95.

Embodiment 184 The method, composition for use, or composition of any one of embodiments 1-88, wherein the sequence selected from SEQ ID NOs:1-146 is SEQ ID NO: 96.

Embodiment 185 The method, composition for use, or composition of any one of embodiments 1-88, wherein the sequence selected from SEQ ID NOs:1-146 is SEQ ID NO: 97.

Embodiment 186 The method, composition for use, or composition of any one of embodiments 1-88, wherein the sequence selected from SEQ ID NOs:1-146 is SEQ ID NO: 98.

Embodiment 187 The method, composition for use, or composition of any one of embodiments 1-88, wherein the sequence selected from SEQ ID NOs:1-146 is SEQ ID NO: 99.

Embodiment 188 The method, composition for use, or composition of any one of embodiments 1-88, wherein the sequence selected from SEQ ID NOs:1-146 is SEQ ID NO: 100.

Embodiment 189 The method, composition for use, or composition of any one of embodiments 1-88, wherein the sequence selected from SEQ ID NOs:1-146 is SEQ ID NO: 101.

Embodiment 190 The method, composition for use, or composition of any one of embodiments 1-88, wherein the sequence selected from SEQ ID NOs:1-146 is SEQ ID NO: 102.

Embodiment 191 The method, composition for use, or composition of any one of embodiments 1-88, wherein the sequence selected from SEQ ID NOs:1-146 is SEQ ID NO: 103.

Embodiment 192 The method, composition for use, or composition of any one of embodiments 1-88, wherein the sequence selected from SEQ ID NOs:1-146 is SEQ ID NO: 104.

Embodiment 193 The method, composition for use, or composition of any one of embodiments 1-88, wherein the sequence selected from SEQ ID NOs:1-146 is SEQ ID NO: 105.

Embodiment 194 The method, composition for use, or composition of any one of embodiments 1-88, wherein the sequence selected from SEQ ID NOs:1-146 is SEQ ID NO: 106.

Embodiment 195 The method, composition for use, or composition of any one of embodiments 1-88, wherein the sequence selected from SEQ ID NOs:1-146 is SEQ ID NO: 107.

Embodiment 196 The method, composition for use, or composition of any one of embodiments 1-88, wherein the sequence selected from SEQ ID NOs:1-146 is SEQ ID NO: 108.

Embodiment 197 The method, composition for use, or composition of any one of embodiments 1-88, wherein the sequence selected from SEQ ID NOs:1-146 is SEQ ID NO: 109.

Embodiment 198 The method, composition for use, or composition of any one of embodiments 1-88, wherein the sequence selected from SEQ ID NOs:1-146 is SEQ ID NO: 110.

Embodiment 199 The method, composition for use, or composition of any one of embodiments 1-88, wherein the sequence selected from SEQ ID NOs:1-146 is SEQ ID NO: 111.

Embodiment 200 The method, composition for use, or composition of any one of embodiments 1-88, wherein the sequence selected from SEQ ID NOs:1-146 is SEQ ID NO: 112.

Embodiment 201 The method, composition for use, or composition of any one of embodiments 1-88, wherein the sequence selected from SEQ ID NOs:1-146 is SEQ ID NO: 113.

Embodiment 202 The method, composition for use, or composition of any one of embodiments 1-88, wherein the sequence selected from SEQ ID NOs:1-146 is SEQ ID NO: 114.

Embodiment 203 The method, composition for use, or composition of any one of embodiments 1-88, wherein the sequence selected from SEQ ID NOs:1-146 is SEQ ID NO: 115.

Embodiment 204 The method, composition for use, or composition of any one of embodiments 1-88, wherein the sequence selected from SEQ ID NOs:1-146 is SEQ ID NO: 116.

Embodiment 205 The method, composition for use, or composition of any one of embodiments 1-88, wherein the sequence selected from SEQ ID NOs:1-146 is SEQ ID NO: 117.

Embodiment 206 The method, composition for use, or composition of any one of embodiments 1-88, wherein the sequence selected from SEQ ID NOs:1-146 is SEQ ID NO: 118.

Embodiment 207 The method, composition for use, or composition of any one of embodiments 1-88, wherein the sequence selected from SEQ ID NOs:1-146 is SEQ ID NO: 119.

Embodiment 208 The method, composition for use, or composition of any one of embodiments 1-88, wherein the sequence selected from SEQ ID NOs:1-146 is SEQ ID NO: 120.

Embodiment 209 The method, composition for use, or composition of any one of embodiments 1-88, wherein the sequence selected from SEQ ID NOs:1-146 is SEQ ID NO: 121.

Embodiment 210 The method, composition for use, or composition of any one of embodiments 1-88, wherein the sequence selected from SEQ ID NOs:1-146 is SEQ ID NO: 122.

Embodiment 211 The method, composition for use, or composition of any one of embodiments 1-88, wherein the sequence selected from SEQ ID NOs:1-146 is SEQ ID NO: 123.

Embodiment 212 The method, composition for use, or composition of any one of embodiments 1-88, wherein the sequence selected from SEQ ID NOs:1-146 is SEQ ID NO: 124.

Embodiment 213 The method, composition for use, or composition of any one of embodiments 1-88, wherein the sequence selected from SEQ ID NOs:1-146 is SEQ ID NO: 125.

Embodiment 214 The method, composition for use, or composition of any one of embodiments 1-88, wherein the sequence selected from SEQ ID NOs:1-146 is SEQ ID NO: 126.

Embodiment 215 The method, composition for use, or composition of any one of embodiments 1-88, wherein the sequence selected from SEQ ID NOs:1-146 is SEQ ID NO: 127.

Embodiment 216 The method, composition for use, or composition of any one of embodiments 1-88, wherein the sequence selected from SEQ ID NOs:1-146 is SEQ ID NO: 128.

Embodiment 217 The method, composition for use, or composition of any one of embodiments 1-88, wherein the sequence selected from SEQ ID NOs:1-146 is SEQ ID NO: 129.

Embodiment 218 The method, composition for use, or composition of any one of embodiments 1-88, wherein the sequence selected from SEQ ID NOs:1-146 is SEQ ID NO: 130.

Embodiment 219 The method, composition for use, or composition of any one of embodiments 1-88, wherein the sequence selected from SEQ ID NOs:1-146 is SEQ ID NO: 131.

Embodiment 220 The method, composition for use, or composition of any one of embodiments 1-88, wherein the sequence selected from SEQ ID NOs:1-146 is SEQ ID NO: 132.

Embodiment 221 The method, composition for use, or composition of any one of embodiments 1-88, wherein the sequence selected from SEQ ID NOs:1-146 is SEQ ID NO: 133.

Embodiment 222 The method, composition for use, or composition of any one of embodiments 1-88, wherein the sequence selected from SEQ ID NOs:1-146 is SEQ ID NO: 134.

Embodiment 223 The method, composition for use, or composition of any one of embodiments 1-88, wherein the sequence selected from SEQ ID NOs:1-146 is SEQ ID NO: 135.

Embodiment 224 The method, composition for use, or composition of any one of embodiments 1-88, wherein the sequence selected from SEQ ID NOs:1-146 is SEQ ID NO: 136.

Embodiment 225 The method, composition for use, or composition of any one of embodiments 1-88, wherein the sequence selected from SEQ ID NOs:1-146 is SEQ ID NO: 137.

Embodiment 226 The method, composition for use, or composition of any one of embodiments 1-88, wherein the sequence selected from SEQ ID NOs:1-146 is SEQ ID NO: 138.

Embodiment 227 The method, composition for use, or composition of any one of embodiments 1-88, wherein the sequence selected from SEQ ID NOs:1-146 is SEQ ID NO: 139.

Embodiment 228 The method, composition for use, or composition of any one of embodiments 1-88, wherein the sequence selected from SEQ ID NOs:1-146 is SEQ ID NO: 140.

Embodiment 229 The method, composition for use, or composition of any one of embodiments 1-88, wherein the sequence selected from SEQ ID NOs:1-146 is SEQ ID NO: 141.

Embodiment 230 The method, composition for use, or composition of any one of embodiments 1-88, wherein the sequence selected from SEQ ID NOs:1-146 is SEQ ID NO: 142.

Embodiment 231 The method, composition for use, or composition of any one of embodiments 1-88, wherein the sequence selected from SEQ ID NOs:1-146 is SEQ ID NO: 143.

Embodiment 232 The method, composition for use, or composition of any one of embodiments 1-88, wherein the sequence selected from SEQ ID NOs:1-146 is SEQ ID NO: 144.

Embodiment 233 The method, composition for use, or composition of any one of embodiments 1-88, wherein the sequence selected from SEQ ID NOs:1-146 is SEQ ID NO: 145.

Embodiment 234 The method, composition for use, or composition of any one of embodiments 1-88, wherein the sequence selected from SEQ ID NOs:1-146 is SEQ ID NO: 146.

Embodiment 235 The method, composition for use, or composition of any one of embodiments 1-88, wherein the sequence selected from SEQ ID NOs:1-146 is SEQ ID NO: 146.

Embodiment 236 The method, composition for use, or composition of any one of embodiments 1-88, wherein the guide RNA is an sgRNA comprising SEQ ID NO: 251.

Embodiment 237 The method, composition for use, or composition of any one of embodiments 1-88, wherein the guide RNA is an sgRNA comprising SEQ ID NO: 252.

Embodiment 238 The method, composition for use, or composition of any one of embodiments 1-88, wherein the guide RNA is an sgRNA comprising SEQ ID NO: 253.

Embodiment 239 The method, composition for use, or composition of any one of embodiments 1-88, wherein the guide RNA is an sgRNA comprising SEQ ID NO: 254.

Embodiment 240 The method, composition for use, or composition of any one of embodiments 1-88, wherein the guide RNA is an sgRNA comprising SEQ ID NO: 255.

Embodiment 241 The method, composition for use, or composition of any one of embodiments 1-88, wherein the guide RNA is an sgRNA comprising SEQ ID NO: 256.

Embodiment 242 The method, composition for use, or composition of any one of embodiments 1-88, wherein the guide RNA is an sgRNA comprising SEQ ID NO: 257.

Embodiment 243 The method, composition for use, or composition of any one of embodiments 1-88, wherein the guide RNA is an sgRNA comprising SEQ ID NO: 258.

Embodiment 244 The method, composition for use, or composition of any one of embodiments 1-88, wherein the guide RNA is an sgRNA comprising SEQ ID NO: 259.

Embodiment 245 The method, composition for use, or composition of any one of embodiments 1-88, wherein the guide RNA is an sgRNA comprising SEQ ID NO: 260.

Embodiment 246 The method or composition of any one of embodiments 1-245, wherein the composition is administered as a single dose.

Embodiment 247 The method or composition of any one of embodiments 1-246, wherein the composition is administered one time.

Embodiment 248 The method or composition of any one of embodiments 246 or 247, wherein the single dose or one time administration:
a. induces a DSB; and/or
b. reduces expression of HAO1 gene; and/or
c. treats or prevents PH1; and/or
d. treats or prevents ESRD caused by PH1; and/or
e. treats or prevents calcium oxalate production and deposition; and/or
f. treats or prevents hyperoxaluria; and/or
g. treats or prevents oxalosis; and/or
h. treats or prevents hematuria; and/or
i. increases serum glycolate concentration; and/or
j. reduces oxylate in urine.

Embodiment 249 The method or composition of embodiment 248, wherein the single dose or one time administration achieves any one or more of a)-j) for 3, 4, 5, 6, 7, 8, 9, 10, 11, 12, 13, 14, or 15 weeks.

Embodiment 250 The method or composition of embodiment 248, wherein the single dose or one time administration achieves a durable effect.

Embodiment 251 The method or composition of any one of embodiments 1-250, further comprising achieving a durable effect.

Embodiment 252 The method or composition of embodiment 251, wherein the durable effect persists at least 1 month, at least 3 months, at least 6 months, at least one year, or at least 5 years.

Embodiment 253 The method or composition of any one one of embodiments 1-252, wherein administration of the composition results in a therapeutically relevant reduction of oxalate in urine.

Embodiment 254 The method or composition of any one of embodiments 1-253, wherein administration of the composition results in urinary oxalate levels within a therapeutic range.

Embodiment 255 The method or composition of any one of embodiments 1-254, wherein administration of the composition results in oxalate levels within 100, 120, or 150% of normal range.

Embodiment 256 Use of a composition or formulation of any of embodiments 9-255 for the preparation of a medicament for treating a human subject having PH1.

Also disclosed is the use of a composition or formulation of any of the foregoing embodiments for the preparation of a medicament for treating a human subject having PH1. Also disclosed are any of the foregoing compositions or formulations for use in treating PH1 or for use in modifying (e.g., forming an indel in, or forming a frameshift or nonsense mutation in) a HAO1 gene.

### BRIEF DESCRIPTION OF THE DRAWINGS

FIGS. 1A-1C show correlations of dgRNA editing % in PHH with HEK293_Cas9 (FIG. 1A), HUH7 (FIG. 1B), and PCH (FIG. 1C) editing.

FIG. 2 shows off-target analysis of certain dgRNAs targeting HAO1.

FIG. 3 shows off-target analysis of certain sgRNAs targeting HAO1.

FIG. 4 shows dose response curves of editing % of certain sgRNAs targeting HAO1 in PHH.

FIG. 5 shows dose response curves of editing % of certain sgRNAs targeting HAO1 in PCH.

FIG. 6 shows Western Blot analysis of HAO1-targeted modified sgRNAs (listed in Table 2) in PHH.

FIG. 7 shows Western Blot analysis of HAO1-targeted modified sgRNAs (listed in Table 2) in PCH.

FIG. 8 shows GO protein quantification values and indel frequency from PHH treated with HAO1-targeting modified sgRNAs (listed in Table 2).

FIG. 9 shows GO protein quantification and indel frequency from PCH treated with HAO1-targeting modified sgRNAs (listed in Table 2).

FIG. 10 shows HAO1 editing percentage for various modified sgRNAs (listed in Table 17) in vivo in mice.

FIG. 11 shows urine oxalate levels after treatment with LNPs comprising a modified sgRNA (G723 listed in Table 17) in vivo in AGT-deficient mice in a 5-week study.

FIG. 12 shows urine oxalate levels after treatment with LNPs comprising a modified sgRNA in vivo in AGT-deficient mice in a 15-week study.

FIG. 13 shows Western Blot analysis after treatment with LNPs comprising a modified sgRNA in vivo in AGT-deficient mice in a 15-week study.

FIG. 14 shows the correlation between the editing and protein levels depicted in Table 20.

FIG. 15 labels the 10 conserved region YA sites in an exemplary sgRNA sequence (SEQ ID NO: 201) from 1 to 10. The numbers 25, 45, 50, 56, 64, 67, and 83 indicate the position of the pyrimidine of YA sites 1, 5, 6, 7, 8, 9, and 10 in an sgRNA with a guide region indicated as (N)x, e.g., wherein x is optionally 20.

FIG. 16 shows an exemplary sgRNA (SEQ ID NO: 401; not all modifications are shown) in a possible secondary structure with labels designating individual nucleotides of the conserved region of the sgRNA, including the lower stem, bulge, upper stem, nexus (the nucleotides of which can be referred to as N1 through N18, respectively, in the 5′ to 3′ direction), hairpin 1, and hairpin 2 regions. A nucleotide between hairpin 1 and hairpin 2 is labeled n. A guide region may be present on an sgRNA and is indicated in this figure as “(N)x” preceding the conserved region of the sgRNA.

### DETAILED DESCRIPTION

Reference will now be made in detail to certain embodiments of the invention, examples of which are illustrated in the accompanying drawings. While the invention is described in conjunction with the illustrated embodiments, it will be understood that they are not intended to limit the invention to those embodiments. On the contrary, the invention is intended to cover all alternatives, modifications, and equivalents, which may be included within the invention as defined by the appended claims and included embodiments.

Before describing the present teachings in detail, it is to be understood that the disclosure is not limited to specific compositions or process steps, as such may vary. It should be noted that, as used in this specification and the appended claims, the singular form “a”, “an” and “the” include plural references unless the context clearly dictates otherwise. Thus, for example, reference to “a conjugate” includes a plurality of conjugates and reference to “a cell” includes a plurality of cells and the like. Numeric ranges are inclusive of the numbers defining the range. Measured and measureable values are understood to be approximate, taking into account significant digits and the error associated with the measurement. Also, the use of “comprise”, “comprises”, “comprising”, “contain”, “contains”, “containing”, “include”, “includes”, and “including” are not intended to be limiting. It is to be understood that both the foregoing general description and detailed description are exemplary and explanatory only and are not restrictive of the teachings.

Unless specifically noted in the specification, embodiments in the specification that recite “comprising” various components are also contemplated as “consisting of” or “consisting essentially of” the recited components; embodiments in the specification that recite “consisting of” various components are also contemplated as “comprising” or “consisting essentially of” the recited components; and embodiments in the specification that recite “consisting essentially of” various components are also contemplated as “consisting of” or “comprising” the recited components (this interchangeability does not apply to the use of these terms in the claims). The term “or” is used in an inclusive sense, i.e., equivalent to “and/or,” unless the context clearly indicates otherwise.

The section headings used herein are for organizational purposes only and are not to be construed as limiting the desired subject matter in any way. In the event that any material incorporated by reference contradicts any term defined in this specification or any other express content of this specification, this specification controls. While the present teachings are described in conjunction with various embodiments, it is not intended that the present teachings be limited to such embodiments. On the contrary, the present teachings encompass various alternatives, modifications, and equivalents, as will be appreciated by those of skill in the art.

### I. Definitions

Unless stated otherwise, the following terms and phrases as used herein are intended to have the following meanings:

“Polynucleotide” and “nucleic acid” are used herein to refer to a multimeric compound comprising nucleosides or nucleoside analogs which have nitrogenous heterocyclic bases or base analogs linked together along a backbone, including conventional RNA, DNA, mixed RNA-DNA, and polymers that are analogs thereof. A nucleic acid “backbone” can be made up of a variety of linkages, including one or more of sugar-phosphodiester linkages, peptide-nucleic acid bonds (“peptide nucleic acids” or PNA; PCT No. WO 95/32305), phosphorothioate linkages, methylphosphonate linkages, or combinations thereof. Sugar moieties of a nucleic acid can be ribose, deoxyribose, or similar compounds with substitutions, e.g., 2′ methoxy or 2′ halide substitutions. Nitrogenous bases can be conventional bases (A, G, C, T, U), analogs thereof (e.g., modified uridines such as 5-methoxyuridine, pseudouridine, or N1-methylpseudouridine, or others); inosine; derivatives of purines or pyrimidines (e.g., N⁴-methyl deoxyguanosine, deaza- or aza-purines, deaza- or aza-pyrimidines, pyrimidine bases with substituent groups at the 5 or 6 position (e.g., 5-methylcytosine), purine bases with a substituent at the 2, 6, or 8 positions, 2-amino-6-methylaminopurine, O⁶-methylguanine, 4-thio-pyrimidines, 4-amino-pyrimidines, 4-dimethylhydrazine-pyrimidines, and O⁴-alkyl-pyrimidines; U.S. Pat. No. 5,378,825 and PCT No. WO 93/13121). For general discussion see The Biochemistry of the Nucleic Acids 5-36, Adams et al., ed., 11^{th} ed., 1992). Nucleic acids can include one or more “abasic” residues where the backbone includes no nitrogenous base for position(s) of the polymer (U.S. Pat. No. 5,585,481). A nucleic acid can comprise only conventional RNA or DNA sugars, bases and linkages, or can include both conventional components and substitutions (e.g., conventional bases with 2′ methoxy linkages, or polymers containing both conventional bases and one or more base analogs). Nucleic acid includes “locked nucleic acid” (LNA), an analogue containing one or more LNA nucleotide monomers with a bicyclic furanose unit locked in an RNA mimicking sugar conformation, which enhance hybridization affinity toward complementary RNA and DNA sequences (Vester and Wengel, 2004, Biochemistry 43(42):13233-41). RNA and DNA have different sugar moieties and can differ by the presence of uracil or analogs thereof in RNA and thymine or analogs thereof in DNA.

“Guide RNA”, “gRNA”, and simply “guide” are used herein interchangeably to refer to either a crRNA (also known as CRISPR RNA), or the combination of a crRNA and a trRNA (also known as tracrRNA). The crRNA and trRNA may be associated as a single RNA molecule (single guide RNA, sgRNA) or in two separate RNA molecules (dual guide RNA, dgRNA). “Guide RNA” or “gRNA” refers to each type. The trRNA may be a naturally-occurring sequence, or a trRNA sequence with modifications or variations compared to naturally-occurring sequences.

As used herein, a “guide sequence” refers to a sequence within a guide RNA that is complementary to a target sequence and functions to direct a guide RNA to a target sequence for binding or modification (e.g., cleavage) by an RNA-guided DNA binding agent. A “guide sequence” may also be referred to as a “targeting sequence,” or a “spacer sequence.” A guide sequence can be 20 base pairs in length, e.g., in the case of Streptococcus pyogenes (i.e., Spy Cas9) and related Cas9 homologs/orthologs. Shorter or longer sequences can also be used as guides, e.g., 15-, 16-, 17-, 18-, 19-, 21-, 22-, 23-, 24-, or 25-nucleotides in length. For example, in some embodiments, the guide sequence comprises at least 17, 18, 19, or 20 contiguous nucleotides of a sequence selected from SEQ ID NOs:1-146. In some embodiments, the target sequence is in a gene or on a chromosome, for example, and is complementary to the guide sequence. In some embodiments, the degree of complementarity or identity between a guide sequence and its corresponding target sequence may be about 75%, 80%, 85%, 90%, 95%, 96%, 97%, 98%, 99%, or 100%. For example, in some embodiments, the guide sequence comprises a sequence with about 75%, 80%, 85%, 90%, 95%, 96%, 97%, 98%, 99%, or 100% identity to at least 17, 18, 19, or 20 contiguous nucleotides of a sequence selected from SEQ ID NOs:1-146. In some embodiments, the guide sequence and the target region may be 100% complementary or identical. In other embodiments, the guide sequence and the target region may contain at least one mismatch. For example, the guide sequence and the target sequence may contain 1, 2, 3, or 4 mismatches, where the total length of the target sequence is at least 17, 18, 19, 20 or more base pairs. In some embodiments, the guide sequence and the target region may contain 1-4 mismatches where the guide sequence comprises at least 17, 18, 19, 20 or more nucleotides. In some embodiments, the guide sequence and the target region may contain 1, 2, 3, or 4 mismatches where the guide sequence comprises 20 nucleotides.

Target sequences for RNA-guided DNA binding agents include both the positive and negative strands of genomic DNA (i.e., the sequence given and the sequence's reverse compliment), as a nucleic acid substrate for an RNA-guided DNA binding agent is a double stranded nucleic acid. Accordingly, where a guide sequence is said to be “complementary to a target sequence”, it is to be understood that the guide sequence may direct a guide RNA to bind to the reverse complement of a target sequence. Thus, in some embodiments, where the guide sequence binds the reverse complement of a target sequence, the guide sequence is identical to certain nucleotides of the target sequence (e.g., the target sequence not including the PAM) except for the substitution of U for T in the guide sequence.

As used herein, an “RNA-guided DNA binding agent” means a polypeptide or complex of polypeptides having RNA and DNA binding activity, or a DNA-binding subunit of such a complex, wherein the DNA binding activity is sequence-specific and depends on the sequence of the RNA. Exemplary RNA-guided DNA binding agents include Cas cleavases/nickases and inactivated forms thereof (“dCas DNA binding agents”). “Cas nuclease”, also called “Cas protein” as used herein, encompasses Cas cleavases, Cas nickases, and dCas DNA binding agents. Cas cleavases/nickases and dCas DNA binding agents include a Csm or Cmr complex of a type III CRISPR system, the Cas10, Csm1, or Cmr2 subunit thereof, a Cascade complex of a type I CRISPR system, the Cas3 subunit thereof, and Class 2 Cas nucleases. As used herein, a “Class 2 Cas nuclease” is a single-chain polypeptide with RNA-guided DNA binding activity. Class 2 Cas nucleases include Class 2 Cas cleavases/nickases (e.g., H840A, D10A, or N863A variants), which further have RNA-guided DNA cleavases or nickase activity, and Class 2 dCas DNA binding agents, in which cleavase/nickase activity is inactivated. Class 2 Cas nucleases include, for example, Cas9, Cpf1, C2c1, C2c2, C2c3, HF Cas9 (e.g., N497A, R661A, Q695A, Q926A variants), HypaCas9 (e.g., N692A, M694A, Q695A, H698A variants), eSPCas9(1.0) (e.g, K810A, K1003A, R1060A variants), and eSPCas9(1.1) (e.g., K848A, K1003A, R1060A variants) proteins and modifications thereof. Cpf1 protein, Zetsche et al., Cell, 163: 1-13 (2015), is homologous to Cas9, and contains a RuvC-like nuclease domain. Cpf1 sequences of Zetsche are incorporated by reference in their entirety. See, e.g., Zetsche, Tables S1 and S3. See, e.g., Makarova et al., Nat Rev Microbiol, 13(11): 722-36 (2015); Shmakov et al., Molecular Cell, 60:385-397 (2015).

As used herein, “ribonucleoprotein” (RNP) or “RNP complex” refers to a guide RNA together with an RNA-guided DNA binding agent, such as a Cas nuclease, e.g., a Cas cleavase, Cas nickase, or dCas DNA binding agent (e.g., Cas9). In some embodiments, the guide RNA guides the RNA-guided DNA binding agent such as Cas9 to a target sequence, and the guide RNA hybridizes with and the agent binds to the target sequence; in cases where the agent is a cleavase or nickase, binding can be followed by cleaving or nicking.

As used herein, a first sequence is considered to “comprise a sequence with at least X % identity to” a second sequence if an alignment of the first sequence to the second sequence shows that X % or more of the positions of the second sequence in its entirety are matched by the first sequence. For example, the sequence AAGA comprises a sequence with 100% identity to the sequence AAG because an alignment would give 100% identity in that there are matches to all three positions of the second sequence. The differences between RNA and DNA (generally the exchange of uridine for thymidine or vice versa) and the presence of nucleoside analogs such as modified uridines do not contribute to differences in identity or complementarity among polynucleotides as long as the relevant nucleotides (such as thymidine, uridine, or modified uridine) have the same complement (e.g., adenosine for all of thymidine, uridine, or modified uridine; another example is cytosine and 5-methylcytosine, both of which have guanosine or modified guanosine as a complement). Thus, for example, the sequence 5′-AXG where X is any modified uridine, such as pseudouridine, N1-methyl pseudouridine, or 5-methoxyuridine, is considered 100% identical to AUG in that both are perfectly complementary to the same sequence (5′-CAU). Exemplary alignment algorithms are the Smith-Waterman and Needleman-Wunsch algorithms, which are well-known in the art. One skilled in the art will understand what choice of algorithm and parameter settings are appropriate for a given pair of sequences to be aligned; for sequences of generally similar length and expected identity >50% for amino acids or >75% for nucleotides, the Needleman-Wunsch algorithm with default settings of the Needleman-Wunsch algorithm interface provided by the EBI at the www.ebi.ac.uk web server is generally appropriate.

“mRNA” is used herein to refer to a polynucleotide that is not DNA and comprises an open reading frame that can be translated into a polypeptide (i.e., can serve as a substrate for translation by a ribosome and amino-acylated tRNAs). mRNA can comprise a phosphate-sugar backbone including ribose residues or analogs thereof, e.g., 2′-methoxy ribose residues. In some embodiments, the sugars of an mRNA phosphate-sugar backbone consist essentially of ribose residues, 2′-methoxy ribose residues, or a combination thereof.

Guide sequences useful in the guide RNA compositions and methods described herein are shown in Table 1 and throughout the application.

As used herein, “indels” refer to insertion/deletion mutations consisting of a number of nucleotides that are either inserted or deleted at the site of double-stranded breaks (DSBs) in a target nucleic acid.

As used herein, “knockdown” refers to a decrease in expression of a particular gene product (e.g., protein, mRNA, or both). Knockdown of a protein can be measured by detecting total cellular amount of the protein from a tissue or cell population of interest. Methods for measuring knockdown of mRNA are known and include sequencing of mRNA isolated from a tissue or cell population of interest. In some embodiments, “knockdown” may refer to some loss of expression of a particular gene product, for example a decrease in the amount of of mRNA transcribed or a decrease in the amount of protein expressed by a population of cells (including in vivo populations such as those found in tissues).

As used herein, “knockout” refers to a loss of expression of a particular protein in a cell. Knockout can be measured either by detecting total cellular amount of a protein in a cell, a tissue or a population of cells. In some embodiments, the methods of the invention “knockout” HAO1 in one or more cells (e.g., in a population of cells including in vivo populations such as those found in tissues). In some embodiments, a knockout is not the formation of mutant HAO1 protein, for example, created by indels, but rather the complete loss of expression of HAO1 protein in a cell. As used herein, “HAO1” refers to hydroxyacid oxidase 1, which is the gene product of a HAO1 gene. The human wild-type HAO1 sequence is available at NCBI Gene ID: 54363; Ensembl: ENSG00000101323. “GOX” and “GOX1” are gene synonyms.

“Primary Hyperoxaluria Type 1 (PH1)” is an an autosomal recessive disorder due to mutation of the AGXT gene, which encodes the liver peroxisomal alanine-glyoxylate aminotransferase (AGT) enzyme. AGT metabolizes glyoxylate to glycine. The lack of AGT activity, or its mistargeting to mitochondria, allows the oxidation of glyoxylate to oxalate, which can only be excreted in the urine. High oxalate levels lead to calcium oxalate stone formation and renal parenchyma damage, which results in progressive deterioration of renal function and, eventually, end-stage renal disease. Thus, a hallmark of PH1 is excessive oxalate production and deposition of calcium oxalate crystals in the kidneys and urinary tract. Renal damage from oxalate is caused by a combination of tubular toxicity, calcium oxalate deposition in the kidneys, and urinary obstruction by calcium oxalate stones. Compromised kidney function exacerbates the disease as the excess oxalate can no longer be effectively excreted, resulting in subsequent accumulation and crystallization of oxalate in bones, eyes, skin, and heart, and other organs leading to severe illness and death. Kidney failure and end stage renal disease are hallmarks. There are no approved pharmaceutical therapies for PH1.

Glycolate oxidase (GO), a hepatic, peroxisomal enzyme upstream of AGT, is one possible mechanism for depleting diseased livers of substrate for oxalate synthesis, to potentially prevent the pathology that develops in PH1. GO, encoded by the hydroxyacid oxidase (HAO1) gene, catalyzes the oxidation of glycolate to glyoxylate. Suppression of GO activity should inhibit oxalate production while causing an accumulation of glycolate. Unlike oxalate, glycolate is soluble and readily excreted in the urine. Thus, in some embodiments, methods for inhibiting GO activity are provided, wherein once inhibited, oxalate production is inhibited and glycolate production is increased.

Oxalate, an oxidation product of glyoxylate, can only be excreted in the urine. High levels of oxalate in the urine (“hyperoxaluria”) is a symptom of PH1. Thus, increased oxalate in the urine is a symptom of PH1. Oxalate can combine with calcium to form calcium oxalate, which is the main component of kidney and bladder stones. Deposits of calcium oxalate in the kidneys and other tissues can lead to blood in the urine (hematuria), urinary track infections, kidney damage, end stage renal disease and others. Over time, oxalate levels in the blood may rise and calcium oxalate may be deposited in other organs throughout the body (oxalosis or systemic oxalosis).

As used herein, a “target sequence” refers to a sequence of nucleic acid in a target gene that has complementarity to the guide sequence of the gRNA. The interaction of the target sequence and the guide sequence directs an RNA-guided DNA binding agent to bind, and potentially nick or cleave (depending on the activity of the agent), within the target sequence.

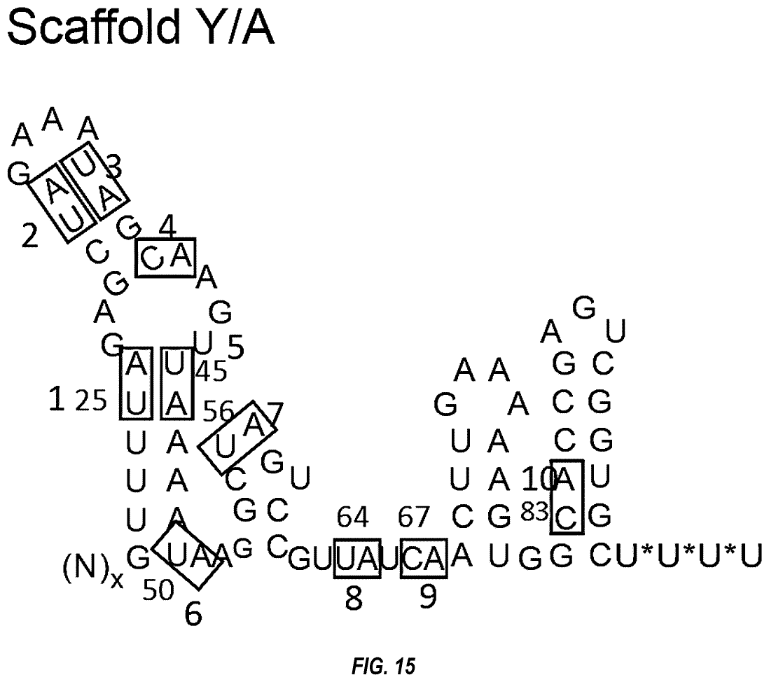

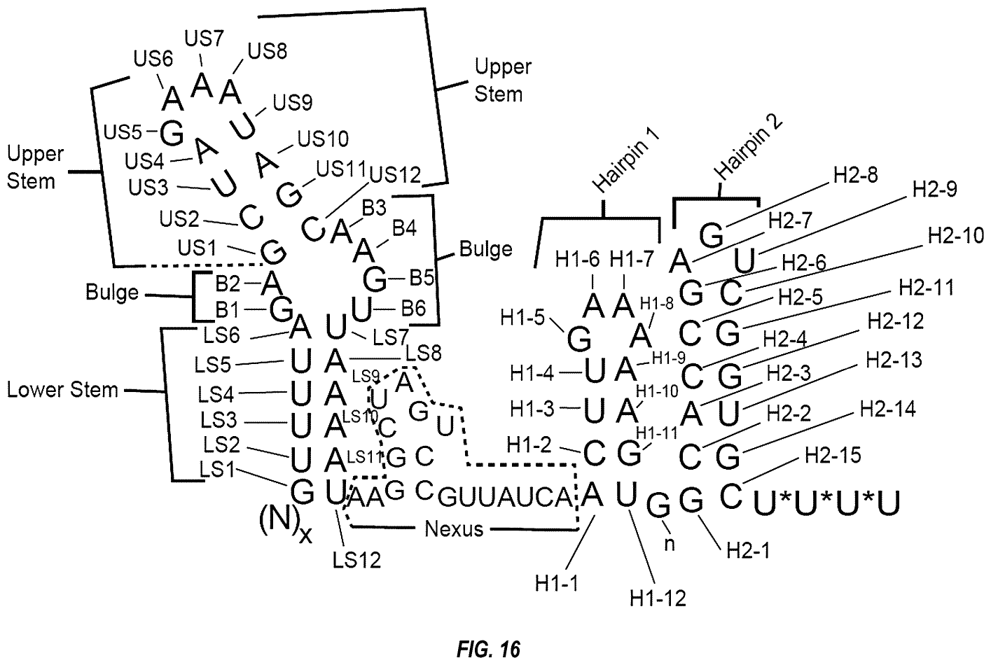

As used herein, a “YA site” refers to a 5′-pyrimidine-adenine-3′ dinucleotide. A “conserved region YA site” is present in the conserved region of an sgRNA. A “guide region YA site” is present in the guide region of an sgRNA. An unmodified YA site in an sgRNA may be susceptible to cleavage by RNase-A like endonucleases, e.g., RNase A. In some embodiments, an sgRNA comprises about 10 YA sites in its conserved region. In some embodiments, an sgRNA comprises 1, 2, 3, 4, 5, 6, 7, 8, 9, or 10 YA sites in its conserved region. Exemplary conserved region YA sites are indicated in FIG. 15. Exemplary guide region YA sites are not shown in FIG. 15, as the guide region may be any sequence, including any number of YA sites. In some embodiments, an sgRNA comprises 1, 2, 3, 4, 5, 6, 7, 8, 9, or 10 of the YA sites indicated in FIG. 15. In some embodiments, an sgRNA comprises 1, 2, 3, 4, 5, 6, 7, 8, 9, or 10 YA sites at the following positions or a subset thereof: LS5-LS6; US3-US4; US9-US10; US12-B3; LS7-LS8; LS12-N1; N6-N7; N14-N15; N17-N18; and H2-2 to H2-3. In some embodiments, a YA site comprises a modification, meaning that at least one nucleotide of the YA site is modified. In some embodiments, the pyrimidine (also called the pyrimidine position) of the YA site comprises a modification (which includes a modification altering the internucleoside linkage immediately 3′ of the sugar of the pyrimidine). In some embodiments, the adenine (also called the adenine position) of the YA site comprises a modification (which includes a modification altering the internucleoside linkage immediately 3′ of the sugar of the adenine). In some embodiments, the pyrimidine position and the adenine position of the YA site comprise modifications.

As used herein, “treatment” refers to any administration or application of a therapeutic for disease or disorder in a subject, and includes inhibiting the disease, arresting its development, relieving one or more symptoms of the disease, curing the disease, or preventing reoccurrence of one or more symptoms of the disease. For example, treatment of PH1 may comprise alleviating symptoms of PH1.

The term “therapeutically relevant reduction of oxalate,” or “oxalate levels within a therapeutic range,” as used herein, means a greater than 30% reduction of urinary oxalate excretion as compared to baseline. See, Leumann and Hoppe (1999) Nephrol Dial Transplant 14:2556-2558 at 2557, second column. For example, achieving oxalate levels within a therapeutic range means reducing urinary oxalate greater than 30% from baseline. In some embodiments, a “normal oxalate level” or a “normal oxalate range” is between about 80 to about 122 μg oxalate/mg creatinine. See, Li et al. (2016) Biochim Biophys Acta 1862(2):233-239. In some embodiments, a therapeutically relevant reduction of oxalate achieves levels of less than or within 200%, 150%, 125%, 120%, 115%, 110%, 105%, or 100% of normal.

The term “about” or “approximately” means an acceptable error for a particular value as determined by one of ordinary skill in the art, which depends in part on how the value is measured or determined.

### II. Compositions

A. Compositions Comprising Guide RNA (gRNAs)

Provided herein are compositions useful for inducing a double-stranded break (DSB) within the HAO1 gene, e.g., using a guide RNA with an RNA-guided DNA binding agent (e.g., a CRISPR/Cas system). The compositions may be administered to subjects having or suspected of having PH1. The compositions may be administered to subjects having increased urinary oxalate output or decreased serum glycolate output. Guide sequences targeting the HAO1 gene are shown in Table 1 at SEQ ID NOs:1-146.

Each of the guide sequences shown in Table 1 at SEQ ID NOs:1-146 may further comprise additional nucleotides to form a crRNA, e.g., with the following exemplary nucleotide sequence following the guide sequence at its 3′ end: GUUUUAGAGCUAUGCUGUUUUG (SEQ ID NO: 200) in 5′ to 3′ orientation. In the case of a sgRNA, the above guide sequences may further comprise additional nucleotides to form a sgRNA, e.g., with the following exemplary nucleotide sequence following the 3′ end of the guide sequence: GUUUUAGAGCUAGAAAUAGCAAGUUAAAAUAAGGCUAGUCCGUUAUCAACUU GAAAAAGUGGCACCGAGUCGGUGCUUUU (SEQ ID NO: 201) or GUUUUAGAGCUAGAAAUAGCAAGUUAAAAUAAGGCUAGUCCGUUAUCAACUU GAAAAAGUGGCACCGAGUCGGUGC (SEQ ID NO: 203, which is SEQ ID NO: 201 without the four terminal U's) in 5′ to 3′ orientation. In some embodiments, the four terminal U's of SEQ ID NO: 201 are not present. In some embodiments, only 1, 2, or 3 of the four terminal U's of SEQ ID NO: 201 are present.

In some embodiments, HAO1 short-single guide RNAs (HAO1 short-sgRNAs) are provided comprising a guide sequence as described herein and a “conserved portion of an sgRNA” comprising a hairpin region, wherein the hairpin region lacks at least 5-10 nucleotides or 6-10 nucleotides. In certain embodiments, a hairpin region of the HAO1 short-single guide RNAs lacks 5-10 nucleotides with reference to the conserved portion of an sgRNA, e.g. nucleotides H1-1 to H2-15 in Table 2B. In certain embodiments, a hairpin 1 region of the HAO1 short-single guide RNAs lacks 5-10 nucleotides with reference to the conserved portion of an sgRNA, e.g. nucleotides H1-1 to H1-12 in Table 2B.

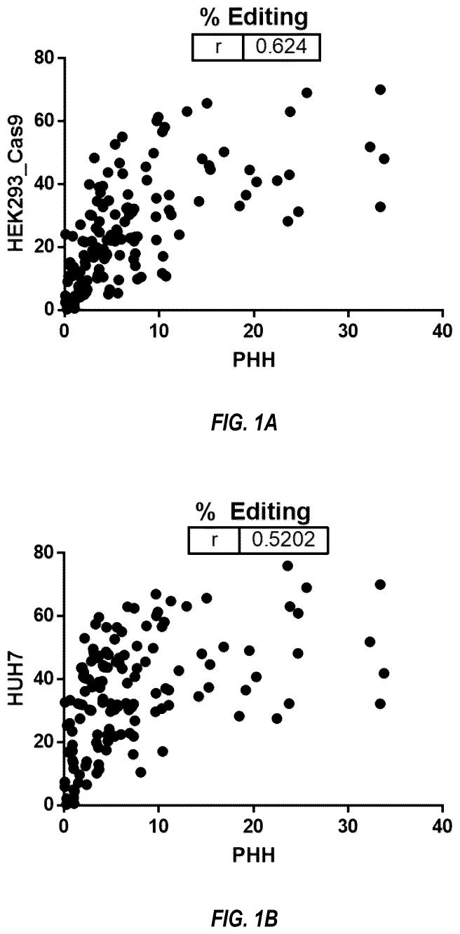

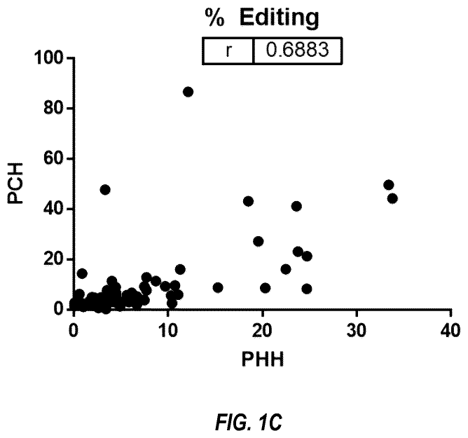

An exemplary “conserved portion of an sgRNA” is shown in Table 2A, which shows a “conserved region” of a S. pyogenes Cas9 (“spyCas9” (also referred to as “spCas9”)) sgRNA. The first row shows the numbering of the nucleotides, the second row shows the sequence (SEQ ID NO: 400); and the third row shows “domains.” Briner A E et al., Molecular Cell 56:333-339 (2014) describes functional domains of sgRNAs, referred to herein as “domains”, including the “spacer” domain responsible for targeting, the “lower stem”, the “bulge”, “upper stem” (which may include a tetraloop), the “nexus”, and the “hairpin 1” and “hairpin 2” domains. See, Briner et al. at page 334, FIG. 1A.

Table 2B provides a schematic of the domains of an sgRNA as used herein. In Table 2B, the “n” between regions represents a variable number of nucleotides, for example, from 0 to 1, 2, 3, 4, 5, 6, 7, 8, 9, 10, 11, 12, 13, 14, 15, 16, 17, 18, 19, 20, or more. In some embodiments, n equals 0. In some embodiments, n equals 1.

In some embodiments, the HAO1 sgRNA is from S. pyogenes Cas9 (“spyCas9”) or a spyCas9 equivalent. In some embodiments, the sgRNA is not from S. pyogenes (“non-spyCas9”). In some embodiments, the 5-10 nucleotides or 6-10 nucleotides are consecutive.

In some embodiments, an HAO1 short-sgRNA lacks at least nucleotides 54-58 (AAAAA) of the conserved portion of a S. pyogenes Cas9 (“spyCas9”) sgRNA, as shown in Table 2A. In some embodiments, an HAO1 short-sgRNA is a non-spyCas9 sgRNA that lacks at least nucleotides corresponding to nucleotides 54-58 (AAAAA) of the conserved portion of a spyCas9 as determined, for example, by pairwise or structural alignment. In some embodiments, the non-spyCas9 sgRNA is Staphylococcus aureus Cas9 (“saCas9”) sgRNA.

In some embodiments, an HAO1 short-sgRNA lacks at least nucleotides 54-61 (AAAAAGUG) of the conserved portion of a spyCas9 sgRNA. In some embodiments, an HAO1 short-sgRNA lacks at least nucleotides 53-60 (GAAAAAGU) of the conserved portion of a spyCas9 sgRNA. In some embodiments, an HAO1 short-sgRNA lacks 4, 5, 6, 7, or 8 nucleotides of nucleotides 53-60 (GAAAAAGU) or nucleotides 54-61 (AAAAAGUG) of the conserved portion of a spyCas9 sgRNA, or the corresponding nucleotides of the conserved portion of a non-spyCas9 sgRNA as determined, for example, by pairwise or structural alignment.

In some embodiments, the sgRNA comprises any one of the guide sequences of SEQ ID Nos: 1-146 and additional nucleotides to form a crRNA, e.g., with the following exemplary nucleotide sequence following the guide sequence at its 3′ end: GUUUUAGAGCUAGAAAUAGCAAGUUAAAAUAAGGCUAGUCCGUUAUCAACUU GGCACCGAGUCGGUGC (SEQ ID NO: 202) in 5′ to 3′ orientation. SEQ ID NO: 202 lacks 8 nucleotides with reference to a wild-type guide RNA conserved sequence:

(SEQ ID NO: 203)
GUUUUAGAGCUAGAAAUAGCAAGUUAAAAUAAGGCUAGUCCGUUAUCAAC
UUGAAAAAGUGGCACCGAGUCGGUGC.

TABLE 1
HAO1 targeted and control guide sequences and chromosomal coordinates

| | | | | SEQ |
|---|---|---|---|---|
| | | | | ID |
| Guide ID | Guide Sequence | Exon | Genomic Coordinates (hg38) | NO: |
| CR002857 | UAAUAGUCAUAUAUAGACUU | Exon 1 | chr20:7940342-7940362 | 1 |
| CR002858 | AAUCAUUGAUACAAAUUAGC | Exon 1 | chr20:7940391-7940411 | 2 |
| CR002859 | AUCAUUGAUACAAAUUAGCC | Exon 1 | chr20:7940392-7940412 | 3 |
| CR002860 | UCAUUGAUACAAAUUAGCCG | Exon 1 | chr20:7940393-7940413 | 4 |
| CR002861 | CAUUGAUACAAAUUAGCCGG | Exon 1 | chr20:7940394-7940414 | 5 |
| CR002862 | AGCCGGGGGAGCAUUUUCAC | Exon 1 | chr20:7940408-7940428 | 6 |
| CR002863 | GGCAAAUGAUGAAGAAACUU | Exon 1 | chr20:7940313-7940333 | 7 |
| CR002864 | AACCUGUGAAAAUGCUCCCC | Exon 1 | chr20:7940413-7940433 | 8 |
| CR002865 | CACAUGAGCCAUGCGCUGCA | Exon 2 | chr20:7934508-7934528 | 9 |
| CR002866 | GCCGUAGCCCCCACACAUAU | Exon 2 | chr20:7934530-7934550 | 10 |
| CR002867 | GAUCUGUUUCAGCAACAUUC | Exon 2 | chr20:7934588-7934608 | 11 |
| CR002868 | GCAACAUUCCGGAGCAUCCU | Exon 2 | chr20:7934599-7934619 | 12 |
| CR002869 | CAUGCAGCGCAUGGCUCAUG | Exon 2 | chr20:7934510-7934530 | 13 |
| CR002870 | GAUCUGUCGACUUCUGUUUU | Exon 2 | chr20:7934572-7934592 | 14 |
| CR002871 | AGCUGUAUCCAAGGAUGCUC | Exon 2 | chr20:7934610-7934630 | 15 |
| CR002872 | CUAGAUGGAAGCUGUAUCCA | Exon 2 | chr20:7934619-7934639 | 16 |
| CR002873 | UGUGUCCACUGUCACAAAUA | Exon 3 | chr20:7914225-7914245 | 17 |
| CR002874 | CGCCACUUCUUCAAUUGAGG | Exon 3 | chr20:7914351-7914371 | 18 |
| CR002875 | CACUUCUUCAAUUGAGGAGG | Exon 3 | chr20:7914354-7914374 | 19 |
| CR002876 | UCAACAUCAUGCCCGUUCCC | Exon 3 | chr20:7914386-7914406 | 20 |
| CR002877 | CAACAUCAUGCCCGUUCCCA | Exon 3 | chr20:7914387-7914407 | 21 |
| CR002878 | GCCCGUUCCCAGGGACUGAC | Exon 3 | chr20:7914396-7914416 | 22 |
| CR002879 | ACAGUGGACACACCUUACCU | Exon 3 | chr20:7914217-7914237 | 23 |
| CR002880 | GACAGUGGACACACCUUACC | Exon 3 | chr20:7914218-7914238 | 24 |
| CR002881 | CAAGGCCAUAUUUGUGACAG | Exon 3 | chr20:7914233-7914253 | 25 |
| CR002882 | CUGGUCCUGAGGCACUUCGU | Exon 3 | chr20:7914327-7914347 | 26 |
| CR002883 | CACCUCCUCAAUUGAAGAAG | Exon 3 | chr20:7914356-7914376 | 27 |
| CR002884 | GGGCAUGAUGUUGAGUUCCU | Exon 3 | chr20:7914380-7914400 | 28 |
| CR002885 | CGGGCAUGAUGUUGAGUUCC | Exon 3 | chr20:7914381-7914401 | 29 |
| CR002886 | GCCUGUCAGUCCCUGGGAAC | Exon 3 | chr20:7914400-7914420 | 30 |
| CR002887 | AGCCUGUCAGUCCCUGGGAA | Exon 3 | chr20:7914401-7914421 | 31 |
| CR002888 | UUCUCAGCCUGUCAGUCCCU | Exon 3 | chr20:7914406-7914426 | 32 |
| CR002889 | UUUCUCAGCCUGUCAGUCCC | Exon 3 | chr20:7914407-7914427 | 33 |
| CR002890 | AAAAUGCCCUUUGCAACAAU | Exon 4 | chr20:7906158-7906178 | 34 |
| CR002891 | AGGAGAAAAUGAUAAAGUAC | Exon 4 | chr20:7906289-7906309 | 35 |
| CR002892 | UCAUUGCCAAUUGUUGCAAA | Exon 4 | chr20:7906167-7906187 | 36 |
| CR002893 | AUCAUUGCCAAUUGUUGCAA | Exon 4 | chr20:7906168-7906188 | 37 |
| CR002894 | UCAGCUGGGAAGAUAUCAAA | Exon 4 | chr20:7906205-7906225 | 38 |
| CR002895 | AAUAGACCCAUCUAUCAGCU | Exon 4 | chr20:7906219-7906239 | 39 |
| CR002896 | CAGUGGACUUGCUGCAUAUG | Exon 4 | chr20:7906249-7906269 | 40 |
| CR002897 | UUUUCUCCUGAGGAAAAUUU | Exon 4 | chr20:7906278-7906298 | 41 |
| CR002898 | UACUUUAUCAUUUUCUCCUG | Exon 4 | chr20:7906288-7906308 | 42 |
| CR002899 | CUGCCAAAACUCACAGUGGC | Exon 5 | chr20:7895118-7895138 | 43 |
| CR002900 | GCCAUGUUUAACAGCCUCCC | Exon 5 | chr20:7895192-7895212 | 44 |
| CR002901 | UGGGGCUCGACAACUCGAUG | Exon 5 | chr20:7895146-7895166 | 45 |
| CR002902 | AUGGGGCUCGACAACUCGAU | Exon 5 | chr20:7895147-7895167 | 46 |
| CR002903 | CAUGGGGCUCGACAACUCGA | Exon 5 | chr20:7895148-7895168 | 47 |
| CR002904 | GAUCUUGGUGUCGAAUCAUG | Exon 5 | chr20:7895164-7895184 | 48 |
| CR002905 | GGAUCUUGGUGUCGAAUCAU | Exon 5 | chr20:7895165-7895185 | 49 |
| CR002906 | GGGAUCUUGGUGUCGAAUCA | Exon 5 | chr20:7895166-7895186 | 50 |
| CR002907 | ACAUGGCUUGAAUGGGAUCU | Exon 5 | chr20:7895179-7895199 | 51 |
| CR002908 | CUGUUAAACAUGGCUUGAAU | Exon 5 | chr20:7895186-7895206 | 52 |
| CR002909 | GCUGUUAAACAUGGCUUGAA | Exon 5 | chr20:7895187-7895207 | 53 |
| CR002910 | GCCAGGGAGGCUGUUAAACA | Exon 5 | chr20:7895196-7895216 | 54 |
| CR002911 | UCCAGGUGAUGAUGCCAGGG | Exon 5 | chr20:7895209-7895229 | 55 |
| CR002912 | CUCUCCAGGUGAUGAUGCCA | Exon 5 | chr20:7895212-7895232 | 56 |
| CR002913 | UCUCUCCAGGUGAUGAUGCC | Exon 5 | chr20:7895213-7895233 | 57 |
| CR002914 | UCUCCCCACAAACACAGCCU | Exon 6 | chr20:7885732-7885752 | 58 |
| CR002915 | CUUUCCGCACACCCCCGUCC | Exon 6 | chr20:7885788-7885808 | 59 |
| CR002916 | GCGCCAAGGCUGUGUUUGUG | Exon 6 | chr20:7885738-7885758 | 60 |
| CR002917 | GGCGCCAAGGCUGUGUUUGU | Exon 6 | chr20:7885739-7885759 | 61 |
| CR002918 | UGGCGCCAAGGCUGUGUUUG | Exon 6 | chr20:7885740-7885760 | 62 |
| CR002919 | AGCUCUGGCUCUUGGCGCCA | Exon 6 | chr20:7885752-7885772 | 63 |
| CR002920 | GUUCUGAAAGCUCUGGCUCU | Exon 6 | chr20:7885760-7885780 | 64 |
| CR002921 | CACUGAUGUUCUGAAAGCUC | Exon 6 | chr20:7885767-7885787 | 65 |
| CR002922 | CUGGACGGGGGUGUGCGGAA | Exon 6 | chr20:7885790-7885810 | 66 |
| CR002923 | UCUUCCUGGACGGGGGUGUG | Exon 6 | chr20:7885795-7885815 | 67 |
| CR002924 | GUGGAAGUCUUCCUGGACGG | Exon 6 | chr20:7885802-7885822 | 68 |
| CR002925 | GGUGGAAGUCUUCCUGGACG | Exon 6 | chr20:7885803-7885823 | 69 |
| CR002926 | AGGUGGAAGUCUUCCUGGAC | Exon 6 | chr20:7885804-7885824 | 70 |
| CR002927 | AAGGUGGAAGUCUUCCUGGA | Exon 6 | chr20:7885805-7885825 | 71 |
| CR002928 | AGGGAAGGUGGAAGUCUUCC | Exon 6 | chr20:7885809-7885829 | 72 |
| CR002929 | UGUUCUGCCAGAAAUUGUGG | Exon 6 | chr20:7885839-7885859 | 73 |
| CR002930 | UGAUGUUCUGCCAGAAAUUG | Exon 6 | chr20:7885842-7885862 | 74 |
| CR002931 | GGAAGAAUUCCGGUUGGCCA | Exon 7 | chr20:7885532-7885552 | 75 |
| CR002932 | AGAUACUAAAGGAAGAAUUC | Exon 7 | chr20:7885542-7885562 | 76 |
| CR002933 | UCACUUGGUUAGGGGGAGAA | Exon 7 | chr20:7885582-7885602 | 77 |
| CR002934 | GCACUGUCAGAUCUUGGAAA | Exon 8 | chr20:7883586-7883606 | 78 |
| CR002935 | CAGAUCUUGGAAACGGCCAA | Exon 8 | chr20:7883593-7883613 | 79 |
| CR002936 | UGUCGAUGACUUUCACAUUC | Exon 8 | chr20:7883636-7883656 | 80 |
| CR002937 | UCAUCGACAAGACAUUGGUG | Exon 8 | chr20:7883625-7883645 | 81 |
| CR002938 | GAAAGUCAUCGACAAGACAU | Exon 8 | chr20:7883630-7883650 | 82 |
| CR006092 | AGUCUAUAUAUGACUAUUAC | Exon 1 | chr20:7940341-7940361 | 83 |
| CR006093 | AUAUAUGACUAUUACAGGUC | Exon 1 | chr20:7940336-7940356 | 84 |
| CR006094 | UAUAUGACUAUUACAGGUCU | Exon 1 | chr20:7940335-7940355 | 85 |
| CR006095 | AUAUGACUAUUACAGGUCUG | Exon 1 | chr20:7940334-7940354 | 86 |
| CR006096 | AAAAAAUAAAUUUUCUUACC | Exon 1 | chr20:7940266-7940286 | 87 |
| CR006097 | UUUUAUUUUUUAAUUCUAGA | Exon 2 | chr20:7934634-7934654 | 88 |
| CR006098 | CGACUUCUGUUUUAGGACAG | Exon 2 | chr20:7934565-7934585 | 89 |
| CR006099 | GACUUCUGUUUUAGGACAGA | Exon 2 | chr20:7934564-7934584 | 90 |
| CR006100 | GGUCAGCAUGCCAAUAUGUG | Exon 2 | chr20:7934543-7934563 | 91 |
| CR006101 | GUCAGCAUGCCAAUAUGUGU | Exon 2 | chr20:7934542-7934562 | 92 |
| CR006102 | UCAGCAUGCCAAUAUGUGUG | Exon 2 | chr20:7934541-7934561 | 93 |
| CR006103 | CAGCAUGCCAAUAUGUGUGG | Exon 2 | chr20:7934540-7934560 | 94 |
| CR006104 | GCCAAUAUGUGUGGGGGCUA | Exon 2 | chr20:7934534-7934554 | 95 |
| CR006105 | GGCUACGGCCAUGCAGCGCA | Exon 2 | chr20:7934519-7934539 | 96 |
| CR006106 | CAGCGCAUGGCUCAUGUGGA | Exon 2 | chr20:7934506-7934526 | 97 |
| CR006107 | CUUCCUCCUACCUCUCACAG | Exon 2 | chr20:7934472-7934492 | 98 |
| CR006108 | UUCAAUUGAGGAGGUGGCCC | Exon 3 | chr20:7914360-7914380 | 99 |
| CR006109 | CUCCUCAAUUGAAGAAGUGG | Exon 3 | chr20:7914353-7914373 | 100 |
| CR006110 | UUCCGCCACUUCUUCAAUUG | Exon 3 | chr20:7914348-7914368 | 101 |
| CR006111 | AUUGAAGAAGUGGCGGAAGC | Exon 3 | chr20:7914346-7914366 | 102 |
| CR006112 | AGUGGCGGAAGCUGGUCCUG | Exon 3 | chr20:7914338-7914358 | 103 |
| CR006113 | UGCAGCCAACGAAGUGCCUC | Exon 3 | chr20:7914319-7914339 | 104 |
| CR006114 | GCUGCAACUGUAUAUCUACA | Exon 3 | chr20:7914305-7914325 | 105 |
| CR006115 | CUAGCUUCUUGGUGACUUCU | Exon 3 | chr20:7914278-7914298 | 106 |
| CR006116 | AAGUCACCAAGAAGCUAGUG | Exon 3 | chr20:7914276-7914296 | 107 |
| CR006117 | CACCAAGAAGCUAGUGCGGC | Exon 3 | chr20:7914272-7914292 | 108 |
| CR006118 | UGCCUGCCGCACUAGCUUCU | Exon 3 | chr20:7914267-7914287 | 109 |
| CR006119 | AGUGCGGCAGGCAGAGAAGA | Exon 3 | chr20:7914260-7914280 | 110 |
| CR006120 | GUGCGGCAGGCAGAGAAGAU | Exon 3 | chr20:7914259-7914279 | 111 |
| CR006121 | GGCAGAGAAGAUGGGCUACA | Exon 3 | chr20:7914251-7914271 | 112 |
| CR006122 | ACCUUACCUGGGCAACCGUC | Exon 3 | chr20:7914206-7914226 | 113 |
| CR006123 | UCCAGACGGUUGCCCAGGUA | Exon 3 | chr20:7914202-7914222 | 114 |
| CR006124 | CAUCAUCCAGACGGUUGCCC | Exon 3 | chr20:7914197-7914217 | 115 |
| CR006125 | UGUUACGCACAUCAUCCAGA | Exon 3 | chr20:7914188-7914208 | 116 |
| CR006126 | CAUGGUUACCUGAGUUGUGG | Exon 3 | chr20:7914154-7914174 | 117 |
| CR006127 | GAUCAUGGUUACCUGAGUUG | Exon 3 | chr20:7914151-7914171 | 118 |
| CR006128 | UCGUCUCCAAAAUUUUCCUC | Exon 4 | chr20:7906269-7906289 | 119 |
| CR006129 | GAAAAUUUUGGAGACGACAG | Exon 4 | chr20:7906266-7906286 | 120 |
| CR006130 | CAAUAGACCCAUCUAUCAGC | Exon 4 | chr20:7906220-7906240 | 121 |
| CR006131 | UAUCUUCCCAGCUGAUAGAU | Exon 4 | chr20:7906210-7906230 | 122 |
| CR006132 | AUAUCUUCCCAGCUGAUAGA | Exon 4 | chr20:7906209-7906229 | 123 |
| CR006133 | GCGUCUGCCAAAACUCACAG | Exon 5 | chr20:7895114-7895134 | 124 |
| CR006134 | GCCAGAAAUUGUGGAGGCUG | Exon 6 | chr20:7885833-7885853 | 125 |
| CR006135 | UCCACAGCCUCCACAAUUUC | Exon 6 | chr20:7885829-7885849 | 126 |
| CR006136 | GAAAUUGUGGAGGCUGUGGA | Exon 6 | chr20:7885829-7885849 | 127 |
| CR006137 | AAAUUGUGGAGGCUGUGGAA | Exon 6 | chr20:7885828-7885848 | 128 |
| CR006138 | UGUGGAGGCUGUGGAAGGGA | Exon 6 | chr20:7885824-7885844 | 129 |
| CR006139 | GGAGGCUGUGGAAGGGAAGG | Exon 6 | chr20:7885821-7885841 | 130 |
| CR006140 | UUGUGGGGAGACCAAUCGUU | Exon 6 | chr20:7885723-7885743 | 131 |
| CR006141 | UGUGGGGAGACCAAUCGUUU | Exon 6 | chr20:7885722-7885742 | 132 |
| CR006142 | GUGGGGAGACCAAUCGUUUG | Exon 6 | chr20:7885721-7885741 | 133 |
| CR006143 | AAAGCUAAGCCCCAAACGAU | Exon 6 | chr20:7885709-7885729 | 134 |
| CR006144 | CAUUUCUUUGUCCAGUUACC | Exon 6 | chr20:7885686-7885706 | 135 |
| CR006145 | UGUAUCUUUUCACUUGGUUA | Exon 7 | chr20:7885591-7885611 | 136 |
| CR006146 | GUAUCUUUUCACUUGGUUAG | Exon 7 | chr20:7885590-7885610 | 137 |
| CR006147 | UAUCUUUUCACUUGGUUAGG | Exon 7 | chr20:7885589-7885609 | 138 |
| CR006148 | AGAUGUCCUCGAGAUACUAA | Exon 7 | chr20:7885553-7885573 | 139 |
| CR006149 | AUUCUUCCUUUAGUAUCUCG | Exon 7 | chr20:7885544-7885564 | 140 |
| CR006150 | ACUAAAGGAAGAAUUCCGGU | Exon 7 | chr20:7885538-7885558 | 141 |
| CR006151 | CACUCAGAGCCAUGGCCAAC | Exon 7 | chr20:7885520-7885540 | 142 |
| CR006152 | AGUCUUACCACUCAGAGCCA | Exon 7 | chr20:7885512-7885532 | 143 |
| CR006153 | AUGUAUGCAUUAUUUUUUCA | Exon 8 | chr20:7883664-7883684 | 144 |
| CR006154 | AUUGGUGAGGAAAAAUCCUU | Exon 8 | chr20:7883612-7883632 | 145 |
| CR006155 | UAUUGUGCACUGUCAGAUCU | Exon 8 | chr20:7883580-7883600 | 146 |

TABLE 2
HAO1 targeted crRNA and sgRNA nomenclature and sequence

| | Guide | | |
|---|---|---|---|
| Guide ID | ID | | |
| (crRNA) | (sgRNA) | sgRNA Sequence - unmodified | sRNA Sequence - modified |
| CR002864 | G009428 | AACCUGUGAAAAUGCUCCCCGUU | mA*mA*mC*CUGUGAAAAUGCUCC |
| | | UUAGAGCUAGAAAUAGCAAGUUA | CCGUUUUAGAmGmCmUmAmGmA |
| | | AAAUAAGGCUAGUCCGUUAUCAA | mAmAmUmAmGmCAAGUUAAAAU |
| | | CUUGAAAAAGUGGCACCGAGUCG | AAGGCUAGUCCGUUAUCAmAmCm |
| | | GUGCUUUU (SEQ ID NO: 151) | UmUmGmAmAmAmAmAmGmUmGm |
| | | | GmCmAmCmCmGmAmGmUmCmGm |
| | | | GmUmGmCmU*mU*mU*mU (SEQ ID |
| | | | NO: 251) |
| CR002929 | G009429 | UGUUCUGCCAGAAAUUGUGGGUU | mU*mG*mU*CUCUGCCAGAAAUUG |
| | | UUAGAGCUAGAAAUAGCAAGUUA | UGGGUUUUAGAmGmCmUmAmGm |
| | | AAAUAAGGCUAGUCCGUUAUCAA | AmAmAmUmAmGmCAAGUUAAAA |
| | | CUUGAAAAAGUGGCACCGAGUCG | UAAGGCUAGUCCGUUAUCAmAmC |
| | | GUGCUUUU (SEQ ID NO: 152) | mUmUmGmAmAmAmAmAmGmUmG |
| | | | mGmCmAmCmCmGmAmGmUmCmG |
| | | | mGmUmGmCmU*mU*mU*mU (SEQ |
| | | | ID NO: 252) |
| CR002878 | G009430 | GCCCGUUCCCAGGGACUGACGUU | mG*mC*mC*CGUUCCCAGGGACUG |
| | | UUAGAGCUAGAAAUAGCAAGUUA | ACGUUUUAGAmGmCmUmAmGmA |
| | | AAAUAAGGCUAGUCCGUUAUCAA | mAmAmUmAmGmCAAGUUAAAAU |
| | | CUUGAAAAAGUGGCACCGAGUCG | AAGGCUAGUCCGUUAUCAmAmCm |
| | | GUGCUUUU (SEQ ID NO: 153) | UmUmGmAmAmAmAmAmGmUmGm |
| | | | GmCmAmCmCmGmAmGmUmCmGm |
| | | | GmUmGmCmU*mU*mU*mU (SEQ ID |
| | | | NO: 253) |
| CR002891 | G009431 | AGGAGAAAAUGAUAAAGUACGUU | mA*mG*mG*AGAAAAUGAUAAAG |
| | | UUAGAGCUAGAAAUAGCAAGUUA | UACGUUUUAGAmGmCmUmAmGm |
| | | AAAUAAGGCUAGUCCGUUAUCAA | AmAmAmUmAmGmCAAGUUAAAA |
| | | CUUGAAAAAGUGGCACCGAGUCG | UAAGGCUAGUCCGUUAUCAmAmC |
| | | GUGCUUUU (SEQ ID NO: 154) | mUmUmGmAmAmAmAmAmGmUmG |
| | | | mGmCmAmCmCmGmAmGmUmCmG |
| | | | mGmUmGmCmU*mU*mU*mU (SEQ |
| | | | ID NO: 254) |
| CR002895 | G009432 | AAUAGACCCAUCUAUCAGCUGUU | mA*mA*mU*AGACCCAUCUAUCAG |
| | | UUAGAGCUAGAAAUAGCAAGUUA | CUGUUUUAGAmGmCmUmAmGmA |
| | | AAAUAAGGCUAGUCCGUUAUCAA | mAmAmUmAmGmCAAGUUAAAAU |
| | | CUUGAAAAAGUGGCACCGAGUCG | AAGGCUAGUCCGUUAUCAmAmCm |
| | | GUGCUUUU (SEQ ID NO: 155) | UmUmGmAmAmAmAmAmGmUmGm |
| | | | GmCmAmCmCmGmAmGmUmCmGm |
| | | | GmUmGmCmU*mU*mU*mU (SEQ ID |
| | | | NO: 255) |
| CR006093 | G009433 | AUAUAUGACUAUUACAGGUCGUU | mA*mU*mA*UAUGACUAUUACAG |
| | | UUAGAGCUAGAAAUAGCAAGUUA | GUCGUUUUAGAmGmCmUmAmGm |
| | | AAAUAAGGCUAGUCCGUUAUCAA | AmAmAmUmAmGmCAAGUUAAAA |
| | | CUUGAAAAAGUGGCACCGAGUCG | UAAGGCUAGUCCGUUAUCAmAmC |
| | | GUGCUUUU (SEQ ID NO: 156) | mUmUmGmAmAmAmAmAmGmUmG |
| | | | mGmCmAmCmCmGmAmGmUmCmG |
| | | | mGmUmGmCmU*mU*mU*mU (SEQ |
| | | | ID NO: 256) |
| CR006109 | G009434 | CUCCUCAAUUGAAGAAGUGGGUU | mC*mU*mC*CUCAAUUGAAGAAGU |
| | | UUAGAGCUAGAAAUAGCAAGUUA | GGGUUUUAGAmGmCmUmAmGmA |
| | | AAAUAAGGCUAGUCCGUUAUCAA | mAmAmUmAmGmCAAGUUAAAAU |
| | | CUUGAAAAAGUGGCACCGAGUCG | AAGGCUAGUCCGUUAUCAmAmCm |
| | | GUGCUUUU (SEQ ID NO: 157) | UmUmGmAmAmAmAmAmGmUmGm |
| | | | GmCmAmCmCmGmAmGmUmCmGm |
| | | | GmUmGmCmU*mU*mU*mU (SEQ ID |
| | | | NO: 257) |
| CR006114 | G009435 | GCUGCAACUGUAUAUCUACAGUU | mG*mC*mU*GCAACUGUAUAUCUA |
| | | UUAGAGCUAGAAAUAGCAAGUUA | CAGUUUUAGAmGmCmUmAmGmA |
| | | AAAUAAGGCUAGUCCGUUAUCAA | mAmAmUmAmGmCAAGUUAAAAU |
| | | CUUGAAAAAGUGGCACCGAGUCG | AAGGCUAGUCCGUUAUCAmAmCm |
| | | GUGCUUUU (SEQ ID NO: 158) | UmUmGmAmAmAmAmAmGmUmGm |
| | | | GmCmAmCmCmGmAmGmUmCmGm |
| | | | GmUmGmCmU*mU*mU*mU (SEQ ID |
| | | | NO: 258) |
| CR006122 | G009436 | ACCUUACCUGGGCAACCGUCGUU | mA*mC*mC*UUACCUGGGCAACCG |
| | | UUAGAGCUAGAAAUAGCAAGUUA | UCGUUUUAGAmGmCmUmAmGmA |
| | | AAAUAAGGCUAGUCCGUUAUCAA | mAmAmUmAmGmCAAGUUAAAAU |
| | | CUUGAAAAAGUGGCACCGAGUCG | AAGGCUAGUCCGUUAUCAmAmCm |
| | | GUGCUUUU (SEQ ID NO: 159) | UmUmGmAmAmAmAmAmGmUmGm |
| | | | GmCmAmCmCmGmAmGmUmCmGm |
| | | | GmUmGmCmU*mU*mU*mU (SEQ ID |
| | | | NO: 259) |
| CR006154 | G009437 | AUUGGUGAGGAAAAAUCCUUGUU | mA*mU*mU*GGUGAGGAAAAAUC |
| | | UUAGAGCUAGAAAUAGCAAGUUA | CUUGUUUUAGAmGmCmUmAmGm |
| | | AAAUAAGGCUAGUCCGUUAUCAA | AmAmAmUmAmGmCAAGUUAAAA |
| | | CUUGAAAAAGUGGCACCGAGUCG | UAAGGCUAGUCCGUUAUCAmAmC |
| | | GUGCUUUU (SEQ ID NO: 160) | mUmUmGmAmAmAmAmAmGmUmG |
| | | | mGmCmAmCmCmGmAmGmUmCmG |
| | | | mGmUmGmCmU*mU*mU*mU (SEQ |
| | | | ID NO: 260) |
| CR002864 | G013964 | AACCUGUGAAAAUGCUCCCCGUU | mA*mA*mC*mCUG*U*fG*fA*fA*fA |
| | | UUAGAGCUAGAAAUAGCAAGUUA | AfUfGCUfCfCCCmGUUUfUAGmAm |
| | | AAAUAAGGCUAGUCCGUUAUCAA | GmCmUmAmGmAmAmAmUmAmGm |
| | | CUUGAAAAAGUGGCACCGAGUCG | CmAmAGUfUmAfAmAfAmUAmAmG |
| | | GUGCUUUU (SEQ ID NO: 161) | mGmCmUmAGUmCmCGUfUAmUmC |
| | | | AmAmCmUmUmGmAmAmAmAmAm |
| | | | GmUmGmGmCmAmCmCmGmAmGm |
| | | | UmCmGmGmUmGmCmU*mU*mU*m |
| | | | U (SEQ ID NO: 261) |
| CR002929 | G013965 | UGUUCUGCCAGAAAUUGUGGGUU | mU*mG*mU*mUCU*G*fC*fC*fA*fG |
| | | UUAGAGCUAGAAAUAGCAAGUUA | AfAfAUUfGfUGGmGUUUfUAGmAm |
| | | AAAUAAGGCUAGUCCGUUAUCAA | GmCmUmAmGmAmAmAmUmAmGm |
| | | CUUGAAAAAGUGGCACCGAGUCG | CmAmAGUfUmAfAmAfAmUAmAmG |
| | | GUGCUUUU (SEQ ID NO: 162) | mGmCmUmAGUmCmCGUfUAmUmC |
| | | | AmAmCmUmUmGmAmAmAmAmAm |
| | | | GmUmGmGmCmAmCmCmGmAmGm |
| | | | UmCmGmGmUmGmCmU*mU*mU*m |
| | | | U (SEQ ID NO: 262) |
| CR002878 | G013966 | GCCCGUUCCCAGGGACUGACGUU | mG*mC*mC*mCGU*U*fC*fC*fC*fA |
| | | UUAGAGCUAGAAAUAGCAAGUUA | GfGfGACfUfGACmGUUUfUAGmAm |
| | | AAAUAAGGCUAGUCCGUUAUCAA | GmCmUmAmGmAmAmAmUmAmGm |
| | | CUUGAAAAAGUGGCACCGAGUCG | CmAmAGUfUmAfAmAfAmUAmAmG |
| | | GUGCUUUU (SEQ ID NO: 163) | mGmCmUmAGUmCmCGUfUAmUmC |
| | | | AmAmCmUmUmGmAmAmAmAmAm |
| | | | GmUmGmGmCmAmCmCmGmAmGm |
| | | | UmCmGmGmUmGmCmU*mU*mU*m |
| | | | U (SEQ ID NO: 263) |
| CR002895 | G013967 | AAUAGACCCAUCUAUCAGCUGUU | mA*mA*mU*mAGA*C*fC*fC*fA*fU |
| | | UUAGAGCUAGAAAUAGCAAGUUA | CfUfAUC*fAfGCUmGUUUfUAGmAm |
| | | AAAUAAGGCUAGUCCGUUAUCAA | GmCmUmAmGmAmAmAmUmAmGm |
| | | CUUGAAAAAGUGGCACCGAGUCG | CmAmAGUfUmAfAmAfAmUAmAmG |
| | | GUGCUUUU (SEQ ID NO: 164) | mGmCmUmAGUmCmCGUfUAmUmC |
| | | | AmAmCmUmUmGmAmAmAmAmAm |
| | | | GmUmGmGmCmAmCmCmGmAmGm |
| | | | UmCmGmGmUmGmCmU*mU*mU*m |
| | | | U (SEQ ID NO: 264) |
| CR002864 | G013968 | AACCUGUGAAAAUGCUCCCCGUU | mA*mA*mC*CUGUGAAAAUGCUCC |
| | | UUAGAGCUAGAAAUAGCAAGUUA | CCGUUUUAGAmGmCmUmAmGmA |
| | | AAAUAAGGCUAGUCCGUUAUCAA | mAmAmUmAmGmCAAGUUAAAAU |
| | | CUUGGCACCGAGUCGGUGC (SEQ | AAGGCUAGUCCGUUAUCAACUUG |
| | | ID NO: 165) | GCACCGAGUCGG*mU*mG*mC |
| | | | (SEQ ID NO: 265) |
| CR002929 | G013969 | UGUUCUGCCAGAAAUUGUGGGUU | mU*mG*mU*UCUGCCAGAAAUUG |
| | | UUAGAGCUAGAAAUAGCAAGUUA | UGGGUUUUAGAmGmCmUmAmGm |
| | | AAAUAAGGCUAGUCCGUUAUCAA | AmAmAmUmAmGmCAAGUUAAAA |
| | | CUUGGCACCGAGUCGGUGC (SEQ | UAAGGCUAGUCCGUUAUCAACUU |
| | | ID NO: 166) | GGCACCGAGUCGG*mU*mG*mC |
| | | | (SEQ ID NO: 266) |
| CR002878 | G013970 | GCCCGUUCCCAGGGACUGACGUU | mG*mC*mC*CGUUCCCAGGGACUG |
| | | UUAGAGCUAGAAAUAGCAAGUUA | ACGUUUUAGAmGmCmUmAmGmA |
| | | AAAUAAGGCUAGUCCGUUAUCAA | mAmAmUmAmGmCAAGUUAAAAU |
| | | CUUGGCACCGAGUCGGUGC (SEQ | AAGGCUAGUCCGUUAUCAACUUG |
| | | ID NO: 167) | GCACCGAGUCGG*mU*mG*mC |
| | | | (SEQ ID NO: 267) |
| CR002895 | G013971 | AAUAGACCCAUCUAUCAGCUGUU | mA*mA*mU*AGACCCAUCUAUCAG |
| | | UUAGAGCUAGAAAUAGCAAGUUA | CUGUUUUAGAmGmCmUmAmGmA |
| | | AAAUAAGGCUAGUCCGUUAUCAA | mAmAmUmAmGmCAAGUUAAAAU |
| | | CUUGGCACCGAGUCGGUGC (SEQ | AAGGCUAGUCCGUUAUCAACUUG |
| | | ID NO: 168) | GCACCGAGUCGG*mU*mG*mC |
| | | | (SEQ ID NO: 268) |

TABLE 2A
(Conserved Portion of a spyCas9 sgRNA; SEQ ID NO: 400)

| 1 | 2 | 3 | 4 | 5 | 6 | 7 | 8 | 9 | 10 | 11 | 12 | 13 | 14 | 15 | 16 |
|---|---|---|---|---|---|---|---|---|---|---|---|---|---|---|---|
| G | U | U | U | U | A | G | A | G | C | U | A | G | A | A | A |

| LS1-LS6 | | | | | B1-B2 | | US1-US12 | | | | | | |
|---|---|---|---|---|---|---|---|---|---|---|---|---|---|
| 17 | 18 | 19 | 20 | 21 | 22 | 23 | 24 | 25 | 26 | 27 | 28 | 29 | 30 |
| U | A | G | C | A | A | G | U | U | A | A | A | A | U |

| | | |
|---|---|---|
| US1-US12 | B2-B6 | LS7-LS12 |

| 31 | 32 | 33 | 34 | 35 | 36 | 37 | 38 | 39 | 40 | 41 | 42 | 43 | 44 | 45 | 46 |
|---|---|---|---|---|---|---|---|---|---|---|---|---|---|---|---|
| A | A | G | G | C | U | A | G | U | C | C | G | U | U | A | U |

Nexus

| 47 | 48 | 49 | 50 | 51 | 52 | 53 | 54 | 55 | 56 | 57 | 58 | 59 | 60 | 61 |
|---|---|---|---|---|---|---|---|---|---|---|---|---|---|---|
| C | A | A | C | U | U | G | A | A | A | A | A | G | U | G |

| | | |
|---|---|---|
| Nexus | H1-1 through H1-12 | N |

| 62 | 63 | 64 | 65 | 66 | 67 | 68 | 69 | 70 | 71 | 72 | 73 | 74 | 75 | 76 |
|---|---|---|---|---|---|---|---|---|---|---|---|---|---|---|
| G | C | A | C | C | G | A | G | U | C | G | G | U | G | C |

H2-1 through H2-15

TABLE 2B

| | LS1-6 | | B1 -2 | | US1-12 | | B3-6 | |
|---|---|---|---|---|---|---|---|---|
| 5′ terminus (n) | lower stem | n | bulge | n | upper stem | n | bulge | n |

| | | | | H1-1 thru | | H2-1 thru | |
|---|---|---|---|---|---|---|---|
| LS7-12 | | N1-18 | | H1-12 | | H2-15 | |
| lower stem | n | nexus | n | hairpin 1 | n | hairpin 2 | 3′ terminus |

In some embodiments, the invention provides a composition comprising one or more guide RNA (gRNA) comprising guide sequences that direct an RNA-guided DNA binding agent, which can be a nuclease (e.g., a Cas nuclease such as Cas9), to a target DNA sequence in HAO1. The gRNA may comprise a crRNA comprising a guide sequence shown in Table 1. The gRNA may comprise a crRNA comprising 17, 18, 19, or 20 contiguous nucleotides of a guide sequence shown in Table 1. In some embodiments, the gRNA comprises a crRNA comprising a sequence with about 75%, 80%, 85%, 90%, 95%, 96%, 97%, 98%, 99%, or 100% identity to at least 17, 18, 19, or 20 contiguous nucleotides of a guide sequence shown in Table 1. In some embodiments, the gRNA comprises a crRNA comprising a sequence with about 75%, 80%, 85%, 90%, 95%, 96%, 97%, 98%, 99%, or 100% identity to a guide sequence shown in Table 1. The gRNA may further comprise a trRNA. In each composition and method embodiment described herein, the crRNA and trRNA may be associated as a single RNA (sgRNA) or may be on separate RNAs (dgRNA). In the context of sgRNAs, the crRNA and trRNA components may be covalently linked, e.g., via a phosphodiester bond or other covalent bond.

In each of the composition, use, and method embodiments described herein, the guide RNA may comprise two RNA molecules as a “dual guide RNA” or “dgRNA”. The dgRNA comprises a first RNA molecule comprising a crRNA comprising, e.g., a guide sequence shown in Table 1, and a second RNA molecule comprising a trRNA. The first and second RNA molecules may not be covalently linked, but may form a RNA duplex via the base pairing between portions of the crRNA and the trRNA.

In each of the composition, use, and method embodiments described herein, the guide RNA may comprise a single RNA molecule as a “single guide RNA” or “sgRNA”. The sgRNA may comprise a crRNA (or a portion thereof) comprising a guide sequence shown in Table 1 covalently linked to a trRNA. The sgRNA may comprise 17, 18, 19, or 20 contiguous nucleotides of a guide sequence shown in Table 1. In some embodiments, the crRNA and the trRNA are covalently linked via a linker. In some embodiments, the sgRNA forms a stem-loop structure via the base pairing between portions of the crRNA and the trRNA. In some embodiments, the crRNA and the trRNA are covalently linked via one or more bonds that are not a phosphodiester bond.

In some embodiments, the trRNA may comprise all or a portion of a trRNA sequence derived from a naturally-occurring CRISPR/Cas system. In some embodiments, the trRNA comprises a truncated or modified wild type trRNA. The length of the trRNA depends on the CRISPR/Cas system used. In some embodiments, the trRNA comprises or consists of 5, 6, 7, 8, 9, 10, 11, 12, 13, 14, 15, 16, 17, 18, 19, 20, 25, 30, 40, 50, 60, 70, 80, 90, 100, or more than 100 nucleotides. In some embodiments, the trRNA may comprise certain secondary structures, such as, for example, one or more hairpin or stem-loop structures, or one or more bulge structures.

In some embodiments, the invention provides a composition comprising one or more guide RNAs comprising a guide sequence of any one of SEQ ID NOs:1-146.

In some embodiments, the invention provides a composition comprising one or more sgRNAs comprising any one of SEQ ID NOs: 151-168 or 251-268.

In one aspect, the invention provides a composition comprising a gRNA that comprises a guide sequence that is at least 99%, 98%, 97%, 96%, 95%, 94%, 93%, 92%, 91%, or 90% identical to any of the nucleic acids of SEQ ID NOs:1-146.

In other embodiments, the composition comprises at least one, e.g., at least two gRNA's comprising guide sequences selected from any two or more of the guide sequences of SEQ ID NOs:1-146. In some embodiments, the composition comprises at least two gRNA's that each comprise a guide sequence at least 99%, 98%, 97%, 96%, 95%, 94%, 93%, 92%, 91%, or 90% identical to any of the nucleic acids of SEQ ID NOs:1-146.

The guide RNA compositions of the present invention are designed to recognize (e.g., hybridize to) a target sequence in the HAO1 gene. For example, the HAO1 target sequence may be recognized and cleaved by a provided Cas cleavase comprising a guide RNA. In some embodiments, an RNA-guided DNA binding agent, such as a Cas cleavase, may be directed by a guide RNA to a target sequence of the HAO1 gene, where the guide sequence of the guide RNA hybridizes with the target sequence and the RNA-guided DNA binding agent, such as a Cas cleavase, cleaves the target sequence.

In some embodiments, the selection of the one or more guide RNAs is determined based on target sequences within the HAO1 gene.

Without being bound by any particular theory, mutations (e.g., frameshift mutations resulting from indels occurring as a result of a nuclease-mediated DSB) in certain regions of the gene may be less tolerable than mutations in other regions of the gene, thus the location of a DSB is an important factor in the amount or type of protein knockdown that may result. In some embodiments, a gRNA complementary or having complementarity to a target sequence within HAO1 is used to direct the RNA-guided DNA binding agent to a particular location in the HAO1 gene. In some embodiments, gRNAs are designed to have guide sequences that are complementary or have complementarity to target sequences in exon 1, exon 3, exon 4, exon 5, exon 6, or exon 8 of HAO1.

In some embodiments, the guide sequence is at least 99%, 98%, 97%, 96%, 95%, 94%, 93%, 92%, 91%, or 90% identical to a target sequence present in the human HAO1 gene. In some embodiments, the target sequence may be complementary to the guide sequence of the guide RNA. In some embodiments, the degree of complementarity or identity between a guide sequence of a guide RNA and its corresponding target sequence may be at least 80%, 85%, 90%, 95%, 96%, 97%, 98%, 99%, or 100%. In some embodiments, the target sequence and the guide sequence of the gRNA may be 100% complementary or identical. In other embodiments, the target sequence and the guide sequence of the gRNA may contain at least one mismatch. For example, the target sequence and the guide sequence of the gRNA may contain 1, 2, 3, or 4 mismatches, where the total length of the guide sequence is 20. In some embodiments, the target sequence and the guide sequence of the gRNA may contain 1-4 mismatches where the guide sequence is 20 nucleotides.

In some embodiments, a composition or formulation disclosed herein comprises an mRNA comprising an open reading frame (ORF) encoding an RNA-guided DNA binding agent, such as a Cas nuclease as described herein. In some embodiments, an mRNA comprising an ORF encoding an RNA-guided DNA binding agent, such as a Cas nuclease, is provided, used, or administered.

B. Modified gRNAs and mRNAs

In some embodiments, the gRNA is chemically modified. A gRNA comprising one or more modified nucleosides or nucleotides is called a “modified” gRNA or “chemically modified” gRNA, to describe the presence of one or more non-naturally and/or naturally occurring components or configurations that are used instead of or in addition to the canonical A, G, C, and U residues. In some embodiments, a modified gRNA is synthesized with a non-canonical nucleoside or nucleotide, is here called “modified.” Modified nucleosides and nucleotides can include one or more of: (i) alteration, e.g., replacement, of one or both of the non-linking phosphate oxygens and/or of one or more of the linking phosphate oxygens in the phosphodiester backbone linkage (an exemplary backbone modification); (ii) alteration, e.g., replacement, of a constituent of the ribose sugar, e.g., of the 2′ hydroxyl on the ribose sugar (an exemplary sugar modification); (iii) wholesale replacement of the phosphate moiety with “dephospho” linkers (an exemplary backbone modification); (iv) modification or replacement of a naturally occurring nucleobase, including with a non-canonical nucleobase (an exemplary base modification); (v) replacement or modification of the ribose-phosphate backbone (an exemplary backbone modification); (vi) modification of the 3′ end or 5′ end of the oligonucleotide, e.g., removal, modification or replacement of a terminal phosphate group or conjugation of a moiety, cap or linker (such 3′ or 5′ cap modifications may comprise a sugar and/or backbone modification); and (vii) modification or replacement of the sugar (an exemplary sugar modification).

Chemical modifications such as those listed above can be combined to provide modified gRNAs and/or mRNAs comprising nucleosides and nucleotides (collectively “residues”) that can have two, three, four, or more modifications. For example, a modified residue can have a modified sugar and a modified nucleobase. In some embodiments, every base of a gRNA is modified, e.g., all bases have a modified phosphate group, such as a phosphorothioate group. In certain embodiments, all, or substantially all, of the phosphate groups of an gRNA molecule are replaced with phosphorothioate groups. In some embodiments, modified gRNAs comprise at least one modified residue at or near the 5′ end of the RNA. In some embodiments, modified gRNAs comprise at least one modified residue at or near the 3′ end of the RNA.

In some embodiments, the gRNA comprises one, two, three or more modified residues. In some embodiments, at least 5% (e.g., at least 5%, at least 10%, at least 15%, at least 20%, at least 25%, at least 30%, at least 35%, at least 40%, at least 45%, at least 50%, at least 55%, at least 60%, at least 65%, at least 70%, at least 75%, at least 80%, at least 85%, at least 90%, at least 95%, or 100%) of the positions in a modified gRNA are modified nucleosides or nucleotides.

Unmodified nucleic acids can be prone to degradation by, e.g., intracellular nucleases or those found in serum. For example, nucleases can hydrolyze nucleic acid phosphodiester bonds. Accordingly, in one aspect the gRNAs described herein can contain one or more modified nucleosides or nucleotides, e.g., to introduce stability toward intracellular or serum-based nucleases. In some embodiments, the modified gRNA molecules described herein can exhibit a reduced innate immune response when introduced into a population of cells, both in vivo and ex vivo. The term “innate immune response” includes a cellular response to exogenous nucleic acids, including single stranded nucleic acids, which involves the induction of cytokine expression and release, particularly the interferons, and cell death.

In some embodiments of a backbone modification, the phosphate group of a modified residue can be modified by replacing one or more of the oxygens with a different substituent. Further, the modified residue, e.g., modified residue present in a modified nucleic acid, can include the wholesale replacement of an unmodified phosphate moiety with a modified phosphate group as described herein. In some embodiments, the backbone modification of the phosphate backbone can include alterations that result in either an uncharged linker or a charged linker with unsymmetrical charge distribution.

Examples of modified phosphate groups include, phosphorothioate, phosphoroselenates, borano phosphates, borano phosphate esters, hydrogen phosphonates, phosphoroamidates, alkyl or aryl phosphonates and phosphotriesters. The phosphorous atom in an unmodified phosphate group is achiral. However, replacement of one of the nonbridging oxygens with one of the above atoms or groups of atoms can render the phosphorous atom chiral. The stereogenic phosphorous atom can possess either the “R” configuration (herein Rp) or the “S” configuration (herein Sp). The backbone can also be modified by replacement of a bridging oxygen, (i.e., the oxygen that links the phosphate to the nucleoside), with nitrogen (bridged phosphoroamidates), sulfur (bridged phosphorothioates) and carbon (bridged methylenephosphonates). The replacement can occur at either linking oxygen or at both of the linking oxygens.

The phosphate group can be replaced by non-phosphorus containing connectors in certain backbone modifications. In some embodiments, the charged phosphate group can be replaced by a neutral moiety. Examples of moieties which can replace the phosphate group can include, without limitation, e.g., methyl phosphonate, hydroxylamino, siloxane, carbonate, carboxymethyl, carbamate, amide, thioether, ethylene oxide linker, sulfonate, sulfonamide, thioformacetal, formacetal, oxime, methyleneimino, methylenemethylimino, methylenehydrazo, methylenedimethylhydrazo and methyleneoxymethylimino.

Scaffolds that can mimic nucleic acids can also be constructed wherein the phosphate linker and ribose sugar are replaced by nuclease resistant nucleoside or nucleotide surrogates. Such modifications may comprise backbone and sugar modifications. In some embodiments, the nucleobases can be tethered by a surrogate backbone. Examples can include, without limitation, the morpholino, cyclobutyl, pyrrolidine and peptide nucleic acid (PNA) nucleoside surrogates.

The modified nucleosides and modified nucleotides can include one or more modifications to the sugar group, i.e. at sugar modification. For example, the 2′ hydroxyl group (OH) can be modified, e.g. replaced with a number of different “oxy” or “deoxy” substituents. In some embodiments, modifications to the 2′ hydroxyl group can enhance the stability of the nucleic acid since the hydroxyl can no longer be deprotonated to form a 2′-alkoxide ion.

Examples of 2′ hydroxyl group modifications can include alkoxy or aryloxy (OR, wherein “R” can be, e.g., alkyl, cycloalkyl, aryl, aralkyl, heteroaryl or a sugar); polyethyleneglycols (PEG), O(CH₂CH₂O)ₙCH₂CH₂OR wherein R can be, e.g., H or optionally substituted alkyl, and n can be an integer from 0 to 20 (e.g., from 0 to 4, from 0 to 8, from 0 to 10, from 0 to 16, from 1 to 4, from 1 to 8, from 1 to 10, from 1 to 16, from 1 to 20, from 2 to 4, from 2 to 8, from 2 to 10, from 2 to 16, from 2 to 20, from 4 to 8, from 4 to 10, from 4 to 16, and from 4 to 20). In some embodiments, the 2′ hydroxyl group modification can be 2′-O-Me. In some embodiments, the 2′ hydroxyl group modification can be a 2′-fluoro modification, which replaces the 2′ hydroxyl group with a fluoride. In some embodiments, the 2′ hydroxyl group modification can include “locked” nucleic acids (LNA) in which the 2′ hydroxyl can be connected, e.g., by a C₁₋₆ alkylene or C₁₋₆ heteroalkylene bridge, to the 4′ carbon of the same ribose sugar, where exemplary bridges can include methylene, propylene, ether, or amino bridges; O-amino (wherein amino can be, e.g., NH₂; alkylamino, dialkylamino, heterocyclyl, arylamino, diarylamino, heteroarylamino, or diheteroarylamino, ethylenediamine, or polyamino) and aminoalkoxy, O(CH₂)ₙ-amino, (wherein amino can be, e.g., NH₂; alkylamino, dialkylamino, heterocyclyl, arylamino, diarylamino, heteroarylamino, or diheteroarylamino, ethylenediamine, or polyamino). In some embodiments, the 2′ hydroxyl group modification can include “unlocked” nucleic acids (UNA) in which the ribose ring lacks the C2′-C3′ bond. In some embodiments, the 2′ hydroxyl group modification can include the methoxyethyl group (MOE), (OCH₂CH₂OCH₃, e.g., a PEG derivative).

“Deoxy” 2′ modifications can include hydrogen (i.e. deoxyribose sugars, e.g., at the overhang portions of partially dsRNA); halo (e.g., bromo, chloro, fluoro, or iodo); amino (wherein amino can be, e.g., NH₂; alkylamino, dialkylamino, heterocyclyl, arylamino, diarylamino, heteroarylamino, diheteroarylamino, or amino acid); NH(CH₂CH₂NH)ₙCH2CH₂— amino (wherein amino can be, e.g., as described herein), —NHC(O)R (wherein R can be, e.g., alkyl, cycloalkyl, aryl, aralkyl, heteroaryl or sugar), cyano; mercapto; alkyl-thio-alkyl; thioalkoxy; and alkyl, cycloalkyl, aryl, alkenyl and alkynyl, which may be optionally substituted with e.g., an amino as described herein.

The sugar modification can comprise a sugar group which may also contain one or more carbons that possess the opposite stereochemical configuration than that of the corresponding carbon in ribose. Thus, a modified nucleic acid can include nucleotides containing e.g., arabinose, as the sugar. The modified nucleic acids can also include abasic sugars. These abasic sugars can also be further modified at one or more of the constituent sugar atoms. The modified nucleic acids can also include one or more sugars that are in the L form, e.g. L-nucleosides.

The modified nucleosides and modified nucleotides described herein, which can be incorporated into a modified nucleic acid, can include a modified base, also called a nucleobase. Examples of nucleobases include, but are not limited to, adenine (A), guanine (G), cytosine (C), and uracil (U). These nucleobases can be modified or wholly replaced to provide modified residues that can be incorporated into modified nucleic acids. The nucleobase of the nucleotide can be independently selected from a purine, a pyrimidine, a purine analog, or pyrimidine analog. In some embodiments, the nucleobase can include, for example, naturally-occurring and synthetic derivatives of a base.

In embodiments employing a dual guide RNA, each of the crRNA and the tracr RNA can contain modifications. Such modifications may be at one or both ends of the crRNA and/or tracr RNA. In embodiments comprising an sgRNA, one or more residues at one or both ends of the sgRNA may be chemically modified, and/or internal nucleosides may be modified, and/or the entire sgRNA may be chemically modified. Certain embodiments comprise a 5′ end modification. Certain embodiments comprise a 3′ end modification.

In some embodiments, the guide RNAs disclosed herein comprise one of the modification patterns disclosed in WO2018/107028 A1, filed Dec. 8, 2017, titled “Chemically Modified Guide RNAs,” the contents of which are hereby incorporated by reference in their entirety. In some embodiments, the guide RNAs disclosed herein comprise one of the structures/modification patterns disclosed in US20170114334, the contents of which are hereby incorporated by reference in their entirety. In some embodiments, the guide RNAs disclosed herein comprise one of the structures/modification patterns disclosed in WO2017/136794, the contents of which are hereby incorporated by reference in their entirety.

### C. YA Modifications

A modification at a YA site (also referred to herein as “YA modification”) can be a modification of the internucleoside linkage, a modification of the base (pyrimidine or adenine), e.g. by chemical modification, substitution, or otherwise, and/or a modification of the sugar (e.g. at the 2′ position, such as 2′-O-alkyl, 2′-F, 2′-moe, 2′-F arabinose, 2′-H (deoxyribose), and the like). In some embodiments, a “YA modification” is any modification that alters the structure of the dinucleotide motif to reduce RNA endonuclease activity, e.g., by interfering with recognition or cleavage of a YA site by an RNase and/or by stabilizing an RNA structure (e.g., secondary structure) that decreases accessibility of a cleavage site to an RNase. See Peacock et al., J Org Chem. 76: 7295-7300 (2011); Behlke, Oligonucleotides 18:305-320 (2008); Ku et al., Adv. Drug Delivery Reviews 104: 16-28 (2016); Ghidini et al., Chem. Commun., 2013, 49, 9036. Peacock et al., Belhke, Ku, and Ghidini provide exemplary modifications suitable as YA modifications. Modifications known to those of skill in the art to reduce endonucleolytic degradation are encompassed. Exemplary 2′ ribose modifications that affect the 2′ hydroxyl group involved in RNase cleavage are 2′-H and 2′-O-alkyl, including 2′-O-Me. Modifications such as bicyclic ribose analogs, UNA, and modified internucleoside linkages of the residues at the YA site can be YA modifications. Exemplary base modifications that can stabilize RNA structures are pseudouridine and 5-methylcytosine. In some embodiments, at least one nucleotide of the YA site is modified. In some embodiments, the pyrimidine (also called “pyrimidine position”) of the YA site comprises a modification (which includes a modification altering the internucleoside linkage immediately 3′ of the sugar of the pyrimidine, a modification of the pyrimidine base, and a modification of the ribose, e.g. at its 2′ position). In some embodiments, the adenine (also called “adenine position”) of the YA site comprises a modification (which includes a modification altering the internucleoside linkage immediately 3′ of the sugar of the pyrimidine, a modification of the pyrimidine base, and a modification of the ribose, e.g. at its 2′ position). In some embodiments, the pyrimidine and the adenine of the YA site comprise modifications. In some embodiments, the YA modification reduces RNA endonuclease activity.

In some embodiments, an sgRNA comprises modifications at 1, 2, 3, 4, 5, 6, 7, 8, 9, 10, 11, 12, 13, 14, 15, 16, or more YA sites. In some embodiments, the pyrimidine of the YA site comprises a modification (which includes a modification altering the internucleoside linkage immediately 3′ of the sugar of the pyrimidine). In some embodiments, the adenine of the YA site comprises a modification (which includes a modification altering the internucleoside linkage immediately 3′ of the sugar of the adenine). In some embodiments, the pyrimidine and the adenine of the YA site comprise modifications, such as sugar, base, or internucleoside linkage modifications. The YA modifications can be any of the types of modifications set forth herein. In some embodiments, the YA modifications comprise one or more of phosphorothioate, 2′-OMe, or 2′-fluoro. In some embodiments, the YA modifications comprise pyrimidine modifications comprising one or more of phosphorothioate, 2′-OMe, or 2′-fluoro. In some embodiments, the YA modification comprises a bicyclic ribose analog (e.g., an LNA, BNA, or ENA) within an RNA duplex region that contains one or more YA sites. In some embodiments, the YA modification comprises a bicyclic ribose analog (e.g., an LNA, BNA, or ENA) within an RNA duplex region that contains a YA site, wherein the YA modification is distal to the YA site.

In some embodiments, the sgRNA comprises a guide region YA site modification. In some embodiments, the guide region comprises 1, 2, 3, 4, 5, or more YA sites (“guide region YA sites”) that may comprise YA modifications. In some embodiments, one or more YA sites located at 5-end, 6-end, 7-end, 8-end, 9-end, or 10-end from the 5′ end of the 5′ terminus (where “5-end”, etc., refers to position 5 to the 3′ end of the guide region, i.e., the most 3′ nucleotide in the guide region) comprise YA modifications. In some embodiments, two or more YA sites located at 5-end, 6-end, 7-end, 8-end, 9-end, or 10-end from the 5′ end of the 5′ terminus comprise YA modifications. In some embodiments, three or more YA sites located at 5-end, 6-end, 7-end, 8-end, 9-end, or 10-end from the 5′ end of the 5′ terminus comprise YA modifications. In some embodiments, four or more YA sites located at 5-end, 6-end, 7-end, 8-end, 9-end, or 10-end from the 5′ end of the 5′ terminus comprise YA modifications. In some embodiments, five or more YA sites located at 5-end, 6-end, 7-end, 8-end, 9-end, or 10-end from the 5′ end of the 5′ terminus comprise YA modifications. A modified guide region YA site comprises a YA modification.

In some embodiments, a modified guide region YA site is within 17, 16, 15, 14, 13, 12, 11, 10, or 9 nucleotides of the 3′ terminal nucleotide of the guide region. For example, if a modified guide region YA site is within 10 nucleotides of the 3′ terminal nucleotide of the guide region and the guide region is 20 nucleotides long, then the modified nucleotide of the modified guide region YA site is located at any of positions 11-20. In some embodiments, a YA modification is located within a YA site 20, 19, 18, 17, 16, 15, 14, 13, 12, 11, 10, 9, 8, 7, 6, 5, 4, 3, 2, or 1 nucleotides from the 3′ terminal nucleotide of the guide region. In some embodiments, a YA modification is located 20, 19, 18, 17, 16, 15, 14, 13, 12, 11, 10, 9, 8, 7, 6, 5, 4, 3, 2, or 1 nucleotides from the 3′ terminal nucleotide of the guide region.

In some embodiments, a modified guide region YA site is at or after nucleotide 4, 5, 6, 7, 8, 9, 10, or 11 from the 5′ end of the 5′ terminus.

In some embodiments, a modified guide region YA site is other than a 5′ end modification. For example, an sgRNA can comprise a 5′ end modification as described herein and further comprise a modified guide region YA site. Alternatively, an sgRNA can comprise an unmodified 5′ end and a modified guide region YA site. Alternatively, an sgRNA can comprise a modified 5′ end and an unmodified guide region YA site.

In some embodiments, a modified guide region YA site comprises a modification that at least one nucleotide located 5′ of the guide region YA site does not comprise. For example, if nucleotides 1-3 comprise phosphorothioates, nucleotide 4 comprises only a 2′-OMe modification, and nucleotide 5 is the pyrimidine of a YA site and comprises a phosphorothioate, then the modified guide region YA site comprises a modification (phosphorothioate) that at least one nucleotide located 5′ of the guide region YA site (nucleotide 4) does not comprise. In another example, if nucleotides 1-3 comprise phosphorothioates, and nucleotide 4 is the pyrimidine of a YA site and comprises a 2′-OMe, then the modified guide region YA site comprises a modification (2′-OMe) that at least one nucleotide located 5′ of the guide region YA site (any of nucleotides 1-3) does not comprise. This condition is also always satisfied if an unmodified nucleotide is located 5′ of the modified guide region YA site.

In some embodiments, the modified guide region YA sites comprise modifications as described for YA sites above.

Additional embodiments of guide region YA site modifications are set forth in the summary above. Any embodiments set forth elsewhere in this disclosure may be combined to the extent feasible with any of the foregoing embodiments.

In some embodiments, the sgRNA comprises a conserved region YA site modification. Conserved region YA sites 1-10 are illustrated in FIG. 15. In some embodiments, 1, 2, 3, 4, 5, 6, 7, 8, 9, or 10 conserved region YA sites comprise modifications.

In some embodiments, conserved region YA sites 1, 8, or 1 and 8 comprise YA modifications. In some embodiments, conserved region YA sites 1, 2, 3, 4, and 10 comprise YA modifications. In some embodiments, YA sites 2, 3, 4, 8, and 10 comprise YA modifications. In some embodiments, conserved region YA sites 1, 2, 3, and 10 comprise YA modifications. In some embodiments, YA sites 2, 3, 8, and 10 comprise YA modifications. In some embodiments, YA sites 1, 2, 3, 4, 8, and 10 comprise YA modifications. In some embodiments, 1, 2, 3, 4, 5, 6, 7, or 8 additional conserved region YA sites comprise YA modifications.

In some embodiments, 1, 2, 3, or 4 of conserved region YA sites 2, 3, 4, and 10 comprise YA modifications. In some embodiments, 1, 2, 3, 4, 5, 6, 7, or 8 additional conserved region YA sites comprise YA modifications.

In some embodiments, the modified conserved region YA sites comprise modifications as described for YA sites above.

Additional embodiments of conserved region YA site modifications are set forth in the summary above. Any embodiments set forth elsewhere in this disclosure may be combined to the extent feasible with any of the foregoing embodiments.

In some embodiments, the sgRNA comprises any of the modification patterns shown below in Table 3, where N is any natural or non-natural nucleotide, and wherein the totality of the N's comprise an HAO1 guide sequence as described herein in Table 1. Table 3 does not depict the guide sequence portion of the sgRNA. The modifications remain as shown in Table 3 despite the substitution of N's for the nucleotides of a guide. That is, although the nucleotides of the guide replace the “N's”, the nucleotides are modified as shown in Table 3.

TABLE 3
HAO1 sgRNA modification patterns. The guide sequence is not shown and will
append the shown sequence at its 5′ end.

| SEQ | | |
|---|---|---|
| ID | | |
| NO | Name | Sequence |
| 400 | G000262-mod | GUUUUAGAGCUAGAAAUAGCAAGUUAAAAUAAGGCUAGUCCGUUAUCAA |
| | only | CUUGAAAAAGUmGmGmCmAmCmCmGmAmGmUmCmGmGmUmGmCmU*mU |
| | | *mU*mU |
| 401 | G000263-mod | GUUUUAGAGCUAGAAAUAGCAAGUUAAAAUAAGGCUAGUCCGUUAUCAm |
| | only | AmCmUmUmGmAmAmAmAmAmGmUmGmGmCmAmCmCmGmAmGmUmCmG |
| | | mGmUmGmCmU*mU*mU*mU |
| 402 | G000264-mod | GUUUUAGAGCUAmGmAmAmAUAGCAAGUUAAAAUAAGGCUAGUCCGUU |
| | only | AUCAACUUGAAAAAGUGGCACCGAGUCGGUGCmU*mU*mU*U |
| 403 | G000265-mod | GUUUUAGAmGmCmUmAGAAAmUmAmGmCAAGUUAAAAUAAGGCUAGUC |
| | only | CGUUAUCAACUUGAAAAAGUGGCACCGAGUCGGUGCmU*mU*mU*U |
| 404 | G000266-mod | GUUUUAGAmGmCmUmAmGmAmAmAmUmAmGmCAAGUUAAAAUAAGGCU |
| | only | AGUCCGUUAUCAACUUGAAAAAGUGGCACCGAGUCGGUGCmU*mU*mU*U |
| 405 | G000267-mod | GUUUUAGAmGmCmUmAmGmAmAmAmUmAmGmCAAGUUAAAAUAAGGCU |
| | only | AGUCCGUUAUCAmAmCmUmUmGmAmAmAmAmAmGmUmGmGmCmAmCmC |
| | | mGmAmGmUmCmGmGmUmGmCmU*mU*mU*mU |
| 406 | G000331- | mGUUUUmAGAmGmCmUmAmGmAmAmAmUmAmGmCAAGUUAAAAUAAG |
| | mod only | GCUAGUCCGUUAUCAmAmCmUmUmGmAmAmAmAmAmGmUmGmGmCmA |
| | | mCmCmGmAmGmUmCmGmGmUmGmCmU*mU*mU*mU |
| 407 | G000332- | fGfUfUfUfUfAGAmGmCmUmAmGmAmAmAmUmAmGmCAAGUUAAAAUAA |
| | mod only | GGCUAGUCCGUUAUCAmAmCmUmUmGmAmAmAmAmAmGmUmGmGmCm |
| | | AmCmCmGmAmGmUmCmGmGmUmGmCmU*mU*mU*mU |
| 408 | G000333- | mGfUfUfUfUmAGAmGmCmUmAmGmAmAmAmUmAmGmCAAGUUAAAAUA |
| | mod only | AGGCUAGUCCGUUAUCAmAmCmUmUmGmAmAmAmAmAmGmUmGmGmC |
| | | mAmCmCmGmAmGmUmCmGmGmUmGmCmU*mU*mU*mU |
| 409 | G000334- | GUUUUAGAmGmCmUmAmGmAmAmAmUmAmGmCAAGUmUmAAAmAmUA |
| | mod only | AGGCUAGUCCGUUAUCAmAmCmUmUmGmAmAmAmAmAmGmUmGmGmC |
| | | mAmCmCmGmAmGmUmCmGmGmUmGmCmU*mU*mU*mU |
| 410 | G000335- | GUUUUAGAmGmCmUmAmGmAmAmAmUmAmGmCAAGUmUmAfAfAmAmU |
| | mod only | AAGGCUAGUCCGUUAUCAmAmCmUmUmGmAmAmAmAmAmGmUmGmGm |
| | | CmAmCmCmGmAmGmUmCmGmGmUmGmCmU*mU*mU*mU |
| 411 | G000336- | GUUUUAGAmGmCmUmAmGmAmAmAmUmAmGmCAAGUfUmAfAmAfAmU |
| | mod only | AAGGCUAGUCCGUUAUCAmAmCmUmUmGmAmAmAmAmAmGmUmGmGm |
| | | CmAmCmCmGmAmGmUmCmGmGmUmGmCmU*mU*mU*mU |
| 412 | G000337- | mGUUUUmAGAmGmCmUmAmGmAmAmAmUmAmGmCAAGUmUmAAAmA |
| | mod only | mUAAGGCUAGUCCGUUAUCAmAmCmUmUmGmAmAmAmAmAmGmUmGm |
| | | GmCmAmCmCmGmAmGmUmCmGmGmUmGmCmU*mU*mU*mU |
| 413 | G000338- | mGUUUUmAGAmGmCmUmAmGmAmAmAmUmAmGmCAAGUmUmAfAfAmA |
| | mod only | mUAAGGCUAGUCCGUUAUCAmAmCmUmUmGmAmAmAmAmAmGmUmGm |
| | | GmCmAmCmCmGmAmGmUmCmGmGmUmGmCmU*mU*mU*mU |
| 414 | G000339- | mGUUUUmAGAmGmCmUmAmGmAmAmAmUmAmGmCAAGUfUmAfAmAfA |
| | mod only | mUAAGGCUAGUCCGUUAUCAmAmCmUmUmGmAmAmAmAmAmGmUmGm |
| | | GmCmAmCmCmGmAmGmUmCmGmGmUmGmCmU*mU*mU*mU |
| 415 | G000340- | fGfUfUfUfUfAGAmGmCmUmAmGmAmAmAmUmAmGmCAAGUmUmAAAmA |
| | mod only | mUAAGGCUAGUCCGUUAUCAmAmCmUmUmGmAmAmAmAmAmGmUmGm |
| | | GmCmAmCmCmGmAmGmUmCmGmGmUmGmCmU*mU*mU*mU |
| 416 | G000341- | fGfUfUfUfUfAGAmGmCmUmAmGmAmAmAmUmAmGmCAAGUmUmAfAfAm |
| | mod only | AmUAAGGCUAGUCCGUUAUCAmAmCmUmUmGmAmAmAmAmAmGmUmG |
| | | mGmCmAmCmCmGmAmGmUmCmGmGmUmGmCmU*mU*mU*mU |
| 417 | G000342- | fGfUfUfUfUfAGAmGmCmUmAmGmAmAmAmUmAmGmCAAGUfUmAfAmAfA |
| | mod only | mUAAGGCUAGUCCGUUAUCAmAmCmUmUmGmAmAmAmAmAmGmUmGm |
| | | GmCmAmCmCmGmAmGmUmCmGmGmUmGmCmU*mU*mU*mU |
| 418 | G000343- | GUUUUAmGmAmGmCmUmAmGmAmAmAmUmAmGmCAAGUUAAAAUAAG |
| | mod only | GCUAGUCCGUUAUCAmAmCmUmUmGmAmAmAmAmAmGmUmGmGmCmA |
| | | mCmCmGmAmGmUmCmGmGmUmGmCmU*mU*mU*mU |
| 419 | G000344- | GUUUUAGAmGmCmUmAmGmAmAmAmUmAmGmCmAmAmGmUUAAAAUA |
| | mod only | AGGCUAGUCCGUUAUCAmAmCmUmUmGmAmAmAmAmAmGmUmGmGmC |
| | | mAmCmCmGmAmGmUmCmGmGmUmGmCmU*mU*mU*mU |
| 420 | G000345- | GUUUUAGAmGmCmUmAmGmAmAmAmUmAmGmCAAGUUAAAAUAAGGC |
| | mod only | UAGUCCGUUfAfUfCfAmAmCmUmUmGmAmAmAmAmAmGmUmGmGmCmA |
| | | mCmCmGmAmGmUmCmGmGmUmGmCmU*mU*mU*mU |
| 421 | G000346- | GUUUUAGAmGmCmUmAmGmAmAmAmUmAmGmCAAGUUAAAAUAAGGC |
| | mod only | UAGUCCGUUAmUmCmAmAmCmUmUmGmAmAmAmAmAmGmUmGmGmCm |
| | | AmCmCmGmAmGmUmCmGmGmUmGmCmU*mU*mU*mU |
| 422 | G000347- | fGfUfUfUfUfAmGmAmGmCmUmAmGmAmAmAmUmAmGmCmAmAmGmUmU |
| | mod only | mAfAfAmAmUAAGGCUAGUCCGUUAmUmCmAmAmCmUmUmGmAmAmAm |
| | | AmAmGmUmGmGmCmAmCmCmGmAmGmUmCmGmGmUmGmCmU*mU*mU |
| | | *mU |
| 423 | G000348- | GUUUUAGAmGmCmUmAmGmAmAmAmUmAmGmCAAGUUAAAAUAAGGC |
| | mod only | UAGUCCGUUAUCAmAmCmUmUmGmAmAmAmAmAmGmUmGmGmCmAmC |
| | | mCmGmAmGmUmCmGmGmUmGmCmUmUmUmU |
| 424 | G000349- | GUUUUAGAmGmCmUmAmGmAmAmAmUmAmGmCAAGUUAAAAUAAGGC |
| | mod only | UAGUCCGUUAUCAmAmCmUmUmGmAmAmAmAmAmGmUmGmGmCmAmC |
| | | mCmGmAmGmUmCmGmGmUmGmCmUmU*mU*mU |
| 425 | G000350- | GUUUUAGAmGmCmUmAmGmAmAmAmUmAmGmCAAGUUAAAAUAAGGC |
| | mod only | UAGUCCGUUAUCAmAmCmUmUmGmAmAmAmAmAmGmUmGmGmCmAmC |
| | | mCmGmAmGfUfCfGfGfUfGfCfU*fU*fU*mU |
| 426 | G000351- | GUUUUAGAmGmCmUmAmGmAmAmAmUmAmGmCAAGUUAAAAUAAGGC |
| | mod only | UAGUCCGUUAUCAfAmCfUmUfGmAfAmAfAmAfGmUfGmGfCmAfCmCfGmA |
| | | fGmUfCmGfGmUfGmCfU*mU*fU*mU |
| 427 | G000352- | mGUUUUmAGAmGmCmUmAmGmAmAmAmUmAmGmCAAGUUAAAAUAAG |
| | mod only | GCUAGUCCGUUAUCAmAmCmUmUmGmAmAmAmAmAmGmUmGmGmCmA |
| | | mCmCmGmAmGmUmCmGmGmUmGmCmU*mU*mU*mU |
| 428 | G000353- | fGfUfUfUfUfAGAmGmCmUmAmGmAmAmAmUmAmGmCAAGUUAAAAUAA |
| | mod only | GGCUAGUCCGUUAUCAmAmCmUmUmGmAmAmAmAmAmGmUmGmGmCm |
| | | AmCmCmGmAmGmUmCmGmGmUmGmCmU*mU*mU*mU |
| 429 | G000354- | mGfUfUfUfUmAGAmGmCmUmAmGmAmAmAmUmAmGmCAAGUUAAAAUA |
| | mod only | AGGCUAGUCCGUUAUCAmAmCmUmUmGmAmAmAmAmAmGmUmGmGmC |
| | | mAmCmCmGmAmGmUmCmGmGmUmGmCmU*mU*mU*mU |
| 430 | G000355- | GUUUUAGAmGmCmUmAmGmAmAmAmUmAmGmCAAGUmUmAAAmAmUA |
| | mod only | AGGCUAGUCCGUUAUCAmAmCmUmUmGmAmAmAmAmAmGmUmGmGmC |
| | | mAmCmCmGmAmGmUmCmGmGmUmGmCmU*mU*mU*mU |
| 431 | G000356- | GUUUUAGAmGmCmUmAmGmAmAmAmUmAmGmCAAGUmUmAfAfAmAmU |
| | mod only | AAGGCUAGUCCGUUAUCAmAmCmUmUmGmAmAmAmAmAmGmUmGmGm |
| | | CmAmCmCmGmAmGmUmCmGmGmUmGmCmU*mU*mU*mU |
| 432 | G000357- | GUUUUAGAmGmCmUmAmGmAmAmAmUmAmGmCAAGUfUmAfAmAfAmU |
| | mod only | AAGGCUAGUCCGUUAUCAmAmCmUmUmGmAmAmAmAmAmGmUmGmGm |
| | | CmAmCmCmGmAmGmUmCmGmGmUmGmCmU*mU*mU*mU |
| 433 | G000358- | mGUUUUmAGAmGmCmUmAmGmAmAmAmUmAmGmCAAGUmUmAAAmA |
| | mod only | mUAAGGCUAGUCCGUUAUCAmAmCmUmUmGmAmAmAmAmAmGmUmGm |
| | | GmCmAmCmCmGmAmGmUmCmGmGmUmGmCmU*mU*mU*mU |
| 434 | G000359- | mGUUUUmAGAmGmCmUmAmGmAmAmAmUmAmGmCAAGUmUmAfAfAmA |
| | mod only | mUAAGGCUAGUCCGUUAUCAmAmCmUmUmGmAmAmAmAmAmGmUmGm |
| | | GmCmAmCmCmGmAmGmUmCmGmGmUmGmCmU*mU*mU*mU |
| 435 | G000360- | mGUUUUmAGAmGmCmUmAmGmAmAmAmUmAmGmCAAGUfUmAfAmAfA |
| | mod only | mUAAGGCUAGUCCGUUAUCAmAmCmUmUmGmAmAmAmAmAmGmUmGm |
| | | GmCmAmCmCmGmAmGmUmCmGmGmUmGmCmU*mU*mU*mU |
| 436 | G000361- | fGfUfUfUfUfAGAmGmCmUmAmGmAmAmAmUmAmGmCAAGUmUmAAAmA |
| | mod only | mUAAGGCUAGUCCGUUAUCAmAmCmUmUmGmAmAmAmAmAmGmUmGm |
| | | GmCmAmCmCmGmAmGmUmCmGmGmUmGmCmU*mU*mU*mU |
| 437 | G000362- | fGfUfUfUfUfAGAmGmCmUmAmGmAmAmAmUmAmGmCAAGUmUmAfAfAm |
| | mod only | AmUAAGGCUAGUCCGUUAUCAmAmCmUmUmGmAmAmAmAmAmGmUmG |
| | | mGmCmAmCmCmGmAmGmUmCmGmGmUmGmCmU*mU*mU*mU |
| 438 | G000363- | fGfUfUfUfUfAGAmGmCmUmAmGmAmAmAmUmAmGmCAAGUfUmAfAmAfA |
| | mod only | mUAAGGCUAGUCCGUUAUCAmAmCmUmUmGmAmAmAmAmAmGmUmGm |
| | | GmCmAmCmCmGmAmGmUmCmGmGmUmGmCmU*mU*mU*mU |
| 439 | G000364- | GUUUUAmGmAmGmCmUmAmGmAmAmAmUmAmGmCAAGUUAAAAUAAG |
| | mod only | GCUAGUCCGUUAUCAmAmCmUmUmGmAmAmAmAmAmGmUmGmGmCmA |
| | | mCmCmGmAmGmUmCmGmGmUmGmCmU*mU*mU*mU |
| 440 | G000365- | GUUUUAGAmGmCmUmAmGmAmAmAmUmAmGmCmAmAmGmUUAAAAUA |
| | mod only | AGGCUAGUCCGUUAUCAmAmCmUmUmGmAmAmAmAmAmGmUmGmGmC |
| | | mAmCmCmGmAmGmUmCmGmGmUmGmCmU*mU*mU*mU |
| 441 | G000366- | GUUUUAGAmGmCmUmAmGmAmAmAmUmAmGmCAAGUUAAAAUAAGGC |
| | mod only | UAGUCCGUUfAfUfCfAmAmCmUmUmGmAmAmAmAmAmGmUmGmGmCmA |
| | | mCmCmGmAmGmUmCmGmGmUmGmCmU*mU*mU*mU |
| 442 | G000367- | GUUUUAGAmGmCmUmAmGmAmAmAmUmAmGmCAAGUUAAAAUAAGGC |
| | mod only | UAGUCCGUUAmUmCmAmAmCmUmUmGmAmAmAmAmAmGmUmGmGmCm |
| | | AmCmCmGmAmGmUmCmGmGmUmGmCmU*mU*mU*mU |
| 443 | G000368- | fGfUfUfUfUfAmGmAmGmCmUmAmGmAmAmAmUmAmGmCmAmAmGmUmU |
| | mod only | mAfAfAmAmUAAGGCUAGUCCGUUAmUmCmAmAmCmUmUmGmAmAmAm |
| | | AmAmGmUmGmGmCmAmCmCmGmAmGmUmCmGmGmUmGmCmU*mU*mU |
| | | *mU |
| 444 | G000369- | GUUUUAGAmGmCmUmAmGmAmAmAmUmAmGmCAAGUUAAAAUAAGGC |
| | mod only | UAGUCCGUUAUCAmAmCmUmUmGmAmAmAmAmAmGmUmGmGmCmAmC |
| | | mCmGmAmGmUmCmGmGmUmGmCmUmUmUmU |
| 445 | G000370- | GUUUUAGAmGmCmUmAmGmAmAmAmUmAmGmCAAGUUAAAAUAAGGC |
| | mod only | UAGUCCGUUAUCAmAmCmUmUmGmAmAmAmAmAmGmUmGmGmCmAmC |
| | | mCmGmAmGmUmCmGmGmUmGmCmUmU*mU*mU |
| 446 | G000371- | GUUUUAGAmGmCmUmAmGmAmAmAmUmAmGmCAAGUUAAAAUAAGGC |
| | mod only | UAGUCCGUUAUCAmAmCmUmUmGmAmAmAmAmAmGmUmGmGmCmAmC |
| | | mCmGmAmGfUfCfGfGfUfGfCfU*fU*fU*mU |
| 447 | G000372- | GUUUUAGAmGmCmUmAmGmAmAmAmUmAmGmCAAGUUAAAAUAAGGC |
| | mod only | UAGUCCGUUAUCAfAmCfUmUfGmAfAmAfAmAfGmUfGmGfCmAfCmCfGmA |
| | | fGmUfCmGfGmUfGmCfU*mU*fU*mU |
| 450 | G013968/ | GUUUUAGAmGmCmUmAmGmAmAmAmUmAmGmCAAGUUAAAAUAAGGCU |
| | G013969/ | AGUCCGUUAUCAACUUGGCACCGAGUCGG*mU*mG*mC |
| | G013970/ | |
| | G013971 - | |
| | mod only | |
| 448 | G013964/ | mN*mN*mN*mNNN*N*fN*fN*fN*fNNfNfNNNfNfNNN |
| | G013965/ | |
| | G013966/ - | |
| | guide region | |
| | mod only | |
| 449 | G013967 - | mN*mN*mN*mNNN*N*fN*fN*fN*fNNfNfNNN*fNfNNN |
| | guide region | |
| | mod only | |

In some embodiments, the modified sgRNA comprises the following sequence: mN*mN*mN*NNNNNNNNNNNNNNNNNGUUUUAGAmGmCmUmAmGmAmAmAmU mAmGmCAAGUUAAAAUAAGGCUAGUCCGUUAUCAmAmCmUmUmGmAmAmAm AmAmGmUmGmGmCmAmCmCmGmAmGmUmCmGmGmUmGmCmU*mU*mU*mU (SEQ ID NO: 300), where “N” may be any natural or non-natural nucleotide, and wherein the totality of N's comprise an HAO1 guide sequence as described in Table 1. For example, encompassed herein is SEQ ID NO: 300, where the N's are replaced with any of the guide sequences disclosed herein in Table 1 (SEQ ID Nos: 1-146).

Any of the modifications described below may be present in the gRNAs and mRNAs described herein.

The terms “mA,” “mC,” “mU,” or “mG” may be used to denote a nucleotide that has been modified with 2′-O-Me.

Modification of 2′-O-methyl can be depicted as follows:

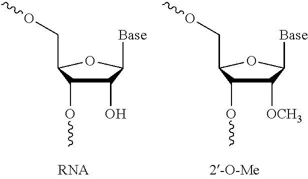

Another chemical modification that has been shown to influence nucleotide sugar rings is halogen substitution. For example, 2′-fluoro (2′-F) substitution on nucleotide sugar rings can increase oligonucleotide binding affinity and nuclease stability.

In this application, the terms “fA,” “fC,” “fU,” or “fG” may be used to denote a nucleotide that has been substituted with 2′-F.

Substitution of 2′-F can be depicted as follows:

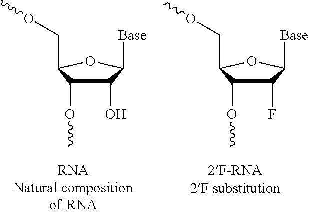

Phosphorothioate (PS) linkage or bond refers to a bond where a sulfur is substituted for one nonbridging phosphate oxygen in a phosphodiester linkage, for example in the bonds between nucleotides bases. When phosphorothioates are used to generate oligonucleotides, the modified oligonucleotides may also be referred to as S-oligos.

A “*” may be used to depict a PS modification. In this application, the terms A*, C*, U*, or G* may be used to denote a nucleotide that is linked to the next (e.g., 3′) nucleotide with a PS bond.

In this application, the terms “mA*,” “mC*,” “mU*,” or “mG*” may be used to denote a nucleotide that has been substituted with 2′-O-Me and that is linked to the next (e.g., 3′) nucleotide with a PS bond.

The diagram below shows the substitution of S— into a nonbridging phosphate oxygen, generating a PS bond in lieu of a phosphodiester bond:

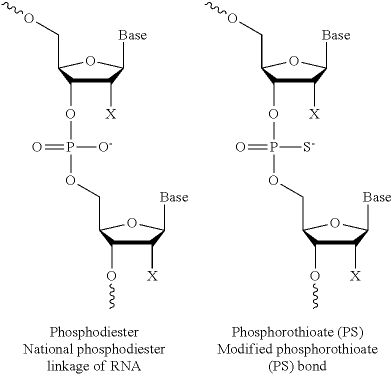

Abasic nucleotides refer to those which lack nitrogenous bases. The figure below depicts an oligonucleotide with an abasic (also known as apurinic) site that lacks a base:

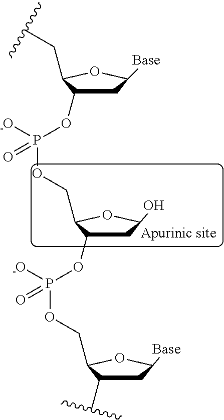

Inverted bases refer to those with linkages that are inverted from the normal 5′ to 3′ linkage (i.e., either a 5′ to 5′ linkage or a 3′ to 3′ linkage). For example:

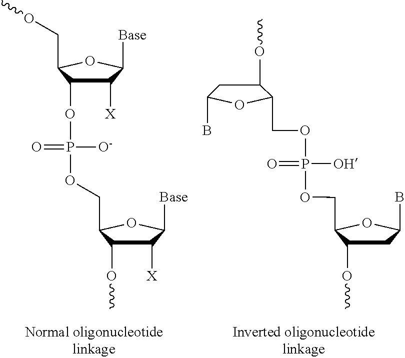

An abasic nucleotide can be attached with an inverted linkage. For example, an abasic nucleotide may be attached to the terminal 5′ nucleotide via a 5′ to 5′ linkage, or an abasic nucleotide may be attached to the terminal 3′ nucleotide via a 3′ to 3′ linkage. An inverted abasic nucleotide at either the terminal 5′ or 3′ nucleotide may also be called an inverted abasic end cap.

In some embodiments, one or more of the first three, four, or five nucleotides at the 5′ terminus, and one or more of the last three, four, or five nucleotides at the 3′ terminus are modified. In some embodiments, the modification is a 2′-O-Me, 2′-F, inverted abasic nucleotide, PS bond, or other nucleotide modification well known in the art to increase stability and/or performance.

In some embodiments, the first four nucleotides at the 5′ terminus, and the last four nucleotides at the 3′ terminus are linked with phosphorothioate (PS) bonds.

In some embodiments, the first three nucleotides at the 5′ terminus, and the last three nucleotides at the 3′ terminus comprise a 2′-O-methyl (2′-O-Me) modified nucleotide. In some embodiments, the first three nucleotides at the 5′ terminus, and the last three nucleotides at the 3′ terminus comprise a 2′-fluoro (2′-F) modified nucleotide. In some embodiments, the first three nucleotides at the 5′ terminus, and the last three nucleotides at the 3′ terminus comprise an inverted abasic nucleotide.

In some embodiments, the guide RNA comprises a modified sgRNA. In some embodiments, the sgRNA comprises the modification pattern shown in SEQ ID No: 201, 202, or 203, where N is any natural or non-natural nucleotide, and where the totality of the N's comprise a guide sequence that directs a nuclease to a target sequence in HAO1, e.g., as shown in Table 1.

In some embodiments, the guide RNA comprises a sgRNA shown in any one of SEQ ID No: 151-168 or 251-268. In some embodiments, the guide RNA comprises a sgRNA comprising any one of the guide sequences of SEQ ID No: 1-146 and the nucleotides of SEQ ID No: 201, 202, or 203, wherein the nucleotides of SEQ ID No: 201, 202, or 203 are on the 3′ end of the guide sequence, and wherein the sgRNA may be modified as shown in Table 3 or SEQ ID NO: 300.

As noted above, in some embodiments, a composition or formulation disclosed herein comprises an mRNA comprising an open reading frame (ORF) encoding an RNA-guided DNA binding agent, such as a Cas nuclease as described herein. In some embodiments, an mRNA comprising an ORF encoding an RNA-guided DNA binding agent, such as a Cas nuclease, is provided, used, or administered. In some embodiments, the ORF encoding an RNA-guided DNA nuclease is a “modified RNA-guided DNA binding agent ORF” or simply a “modified ORF,” which is used as shorthand to indicate that the ORF is modified.

In some embodiments, the modified ORF may comprise a modified uridine at least at one, a plurality of, or all uridine positions. In some embodiments, the modified uridine is a uridine modified at the 5 position, e.g., with a halogen, methyl, or ethyl. In some embodiments, the modified uridine is a pseudouridine modified at the 1 position, e.g., with a halogen, methyl, or ethyl. The modified uridine can be, for example, pseudouridine, N1-methyl-pseudouridine, 5-methoxyuridine, 5-iodouridine, or a combination thereof. In some embodiments, the modified uridine is 5-methoxyuridine. In some embodiments, the modified uridine is 5-iodouridine. In some embodiments, the modified uridine is pseudouridine. In some embodiments, the modified uridine is N1-methyl-pseudouridine. In some embodiments, the modified uridine is a combination of pseudouridine and N1-methyl-pseudouridine. In some embodiments, the modified uridine is a combination of pseudouridine and 5-methoxyuridine. In some embodiments, the modified uridine is a combination of N1-methyl pseudouridine and 5-methoxyuridine. In some embodiments, the modified uridine is a combination of 5-iodouridine and N1-methyl-pseudouridine. In some embodiments, the modified uridine is a combination of pseudouridine and 5-iodouridine. In some embodiments, the modified uridine is a combination of 5-iodouridine and 5-methoxyuridine.

In some embodiments, an mRNA disclosed herein comprises a 5′ cap, such as a Cap0, Cap1, or Cap2. A 5′ cap is generally a 7-methylguanine ribonucleotide (which may be further modified, as discussed below e.g. with respect to ARCA) linked through a 5′-triphosphate to the 5′ position of the first nucleotide of the 5′-to-3′ chain of the mRNA, i.e., the first cap-proximal nucleotide. In Cap0, the riboses of the first and second cap-proximal nucleotides of the mRNA both comprise a 2′-hydroxyl. In Cap1, the riboses of the first and second transcribed nucleotides of the mRNA comprise a 2′-methoxy and a 2′-hydroxyl, respectively. In Cap2, the riboses of the first and second cap-proximal nucleotides of the mRNA both comprise a 2′-methoxy. See, e.g., Katibah et al. (2014) Proc Natl Acad Sci USA 111(33):12025-30; Abbas et al. (2017) Proc Natl Acad Sci USA 114(11):E2106-E2115. Most endogenous higher eukaryotic mRNAs, including mammalian mRNAs such as human mRNAs, comprise Cap1 or Cap2. Cap0 and other cap structures differing from Cap1 and Cap2 may be immunogenic in mammals, such as humans, due to recognition as “non-self” by components of the innate immune system such as IFIT-1 and IFIT-5, which can result in elevated cytokine levels including type I interferon. Components of the innate immune system such as IFIT-1 and IFIT-5 may also compete with eIF4E for binding of an mRNA with a cap other than Cap1 or Cap2, potentially inhibiting translation of the mRNA.

A cap can be included co-transcriptionally. For example, ARCA (anti-reverse cap analog; Thermo Fisher Scientific Cat. No. AM8045) is a cap analog comprising a 7-methylguanine 3′-methoxy-5′-triphosphate linked to the 5′ position of a guanine ribonucleotide which can be incorporated in vitro into a transcript at initiation. ARCA results in a Cap0 cap in which the 2′ position of the first cap-proximal nucleotide is hydroxyl. See, e.g., Stepinski et al., (2001) “Synthesis and properties of mRNAs containing the novel ‘anti-reverse’ cap analogs 7-methyl(3′-O-methyl)GpppG and 7-methyl(3′deoxy)GpppG,” RNA 7: 1486-1495. The ARCA structure is shown below.

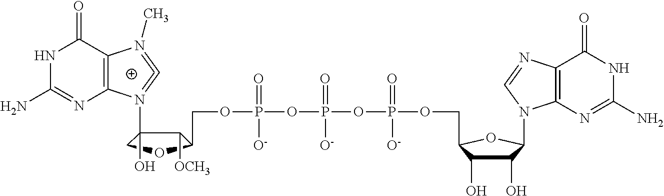

CleanCap™ AG (m7G(5′)ppp(5′)(2′OMeA)pG; TriLink Biotechnologies Cat. No. N-7113) or CleanCap™ GG (m7G(5′)ppp(5′)(2′OMeG)pG; TriLink Biotechnologies Cat. No. N-7133) can be used to provide a Cap1 structure co-transcriptionally. 3′-O-methylated versions of CleanCap™ AG and CleanCap™ GG are also available from TriLink Biotechnologies as Cat. Nos. N-7413 and N-7433, respectively. The CleanCap™ AG structure is shown below.

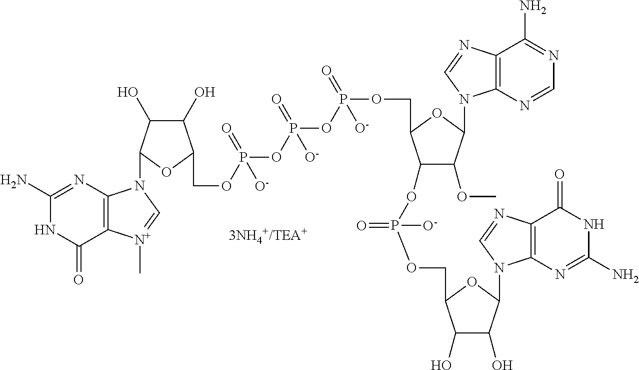 [SMILES: COC1C(OP(=O)([O-])OCC2OC(n3cnc4c(=O)[nH]c(N)nc43)C(O)C2O)C(COP(=O)([O-])OP(=O)([O-])OP(=O)([O-])OCC2OC(n3c[n+](C)c4c(=O)[nH]c(N)nc43)C(O)C2O)OC1n1cnc2c(N)ncnc21]

Alternatively, a cap can be added to an RNA post-transcriptionally. For example, Vaccinia capping enzyme is commercially available (New England Biolabs Cat. No. M2080S) and has RNA triphosphatase and guanylyltransferase activities, provided by its D1 subunit, and guanine methyltransferase, provided by its D12 subunit. As such, it can add a 7-methylguanine to an RNA, so as to give Cap0, in the presence of S-adenosyl methionine and GTP. See, e.g., Guo, P. and Moss, B. (1990) Proc. Natl. Acad. Sci. USA 87, 4023-4027; Mao, X. and Shuman, S. (1994) J. Biol. Chem. 269, 24472-24479.

In some embodiments, the mRNA further comprises a poly-adenylated (poly-A) tail. In some embodiments, the poly-A tail comprises at least 20, 30, 40, 50, 60, 70, 80, 90, or 100 adenines, optionally up to 300 adenines. In some embodiments, the poly-A tail comprises 95, 96, 97, 98, 99, or 100 adenine nucleotides.

### D. Ribonucleoprotein Complex

In some embodiments, a composition is encompassed comprising one or more gRNAs comprising one or more guide sequences from Table 1 or one or more sgRNAs from Table 2 and an RNA-guided DNA binding agent, e.g., a nuclease, such as a Cas nuclease, such as Cas9. In some embodiments, the RNA-guided DNA-binding agent has cleavase activity, which can also be referred to as double-strand endonuclease activity. In some embodiments, the RNA-guided DNA-binding agent comprises a Cas nuclease. Examples of Cas9 nucleases include those of the type II CRISPR systems of S. pyogenes, S. aureus, and other prokaryotes (see, e.g., the list in the next paragraph), and modified (e.g., engineered or mutant) versions thereof. See, e.g., US2016/0312198 A1; US 2016/0312199 A1. Other examples of Cas nucleases include a Csm or Cmr complex of a type III CRISPR system or the Cas10, Csm1, or Cmr2 subunit thereof; and a Cascade complex of a type I CRISPR system, or the Cas3 subunit thereof. In some embodiments, the Cas nuclease may be from a Type-IIA, Type-IIB, or Type-IIC system. For discussion of various CRISPR systems and Cas nucleases see, e.g., Makarova et al., NAT. REV. MICROBIOL. 9:467-477 (2011); Makarova et al., NAT. REV. MICROBIOL, 13: 722-36 (2015); Shmakov et al., MOLECULAR CELL, 60:385-397 (2015).

Non-limiting exemplary species that the Cas nuclease can be derived from include Streptococcus pyogenes, Streptococcus thermophilus, Streptococcus sp., Staphylococcus aureus, Listeria innocua, Lactobacillus gasseri, Francisella novicida, Wolinella succinogenes, Sutterella wadsworthensis, Gammaproteobacterium, Neisseria meningitidis, Campylobacter jejuni, Pasteurella multocida, Fibrobacter succinogene, Rhodospirillum rubrum, Nocardiopsis dassonvillei, Streptomyces pristinaespiralis, Streptomyces viridochromogenes, Streptomyces viridochromogenes, Streptosporangium roseum, Streptosporangium roseum, Alicyclobacillus acidocaldarius, Bacillus pseudomycoides, Bacillus selenitireducens, Exiguobacterium sibiricum, Lactobacillus delbrueckii, Lactobacillus salivarius, Lactobacillus buchneri, Treponema denticola, Microscilla marina, Burkholderiales bacterium, Polaromonas naphthalenivorans, Polaromonas sp., Crocosphaera watsonii, Cyanothece sp., Microcystis aeruginosa, Synechococcus sp., Acetohalobium arabaticum, Ammonifex degensii, Caldicelulosiruptor becscii, Candidatus Desulforudis, Clostridium botulinum, Clostridium difficile, Finegoldia magna, Natranaerobius thermophilus, Pelotomaculum thermopropionicum, Acidithiobacillus caldus, Acidithiobacillus ferrooxidans, Allochromatium vinosum, Marinobacter sp., Nitrosococcus halophilus, Nitrosococcus watsoni, Pseudoalteromonas haloplanktis, Ktedonobacter racemifer, Methanohalobium evestigatum, Anabaena variabilis, Nodularia spumigena, Nostoc sp., Arthrospira maxima, Arthrospira platensis, Arthrospira sp., Lyngbya sp., Microcoleus chthonoplastes, Oscillatoria sp., Petrotoga mobilis, Thermosipho africanus, Streptococcus pasteurianus, Neisseria cinerea, Campylobacter lari, Parvibaculum lavamentivorans, Corynebacterium diphtheria, Acidaminococcus sp., Lachnospiraceae bacterium ND2006, and Acaryochloris marina.

In some embodiments, the Cas nuclease is the Cas9 nuclease from Streptococcus pyogenes. In some embodiments, the Cas nuclease is the Cas9 nuclease from Streptococcus thermophilus. In some embodiments, the Cas nuclease is the Cas9 nuclease from Neisseria meningitidis. In some embodiments, the Cas nuclease is the Cas9 nuclease is from Staphylococcus aureus. In some embodiments, the Cas nuclease is the Cpf1 nuclease from Francisella novicida. In some embodiments, the Cas nuclease is the Cpf1 nuclease from Acidaminococcus sp. In some embodiments, the Cas nuclease is the Cpf1 nuclease from Lachnospiraceae bacterium ND2006. In further embodiments, the Cas nuclease is the Cpf1 nuclease from Francisella tularensis, Lachnospiraceae bacterium, Butyrivibrio proteoclasticus, Peregrinibacteria bacterium, Parcubacteria bacterium, Smithella, Acidaminococcus, Candidatus Methanoplasma termitum, Eubacterium eligens, Moraxella bovoculi, Leptospira inadai, Porphyromonas crevioricanis, Prevotella disiens, or Porphyromonas macacae. In certain embodiments, the Cas nuclease is a Cpf1 nuclease from an Acidaminococcus or Lachnospiraceae.

In some embodiments, the gRNA together with an RNA-guided DNA binding agent is called a ribonucleoprotein complex (RNP). In some embodiments, the RNA-guided DNA binding agent is a Cas nuclease. In some embodiments, the gRNA together with a Cas nuclease is called a Cas RNP. In some embodiments, the RNP comprises Type-I, Type-II, or Type-III components. In some embodiments, the Cas nuclease is the Cas9 protein from the Type-II CRISPR/Cas system. In some embodiment, the gRNA together with Cas9 is called a Cas9 RNP.

Wild type Cas9 has two nuclease domains: RuvC and HNH. The RuvC domain cleaves the non-target DNA strand, and the HNH domain cleaves the target strand of DNA. In some embodiments, the Cas9 protein comprises more than one RuvC domain and/or more than one HNH domain. In some embodiments, the Cas9 protein is a wild type Cas9. In each of the composition, use, and method embodiments, the Cas induces a double strand break in target DNA.

In some embodiments, chimeric Cas nucleases are used, where one domain or region of the protein is replaced by a portion of a different protein. In some embodiments, a Cas nuclease domain may be replaced with a domain from a different nuclease such as Fok1. In some embodiments, a Cas nuclease may be a modified nuclease.

In other embodiments, the Cas nuclease may be from a Type-I CRISPR/Cas system. In some embodiments, the Cas nuclease may be a component of the Cascade complex of a Type-I CRISPR/Cas system. In some embodiments, the Cas nuclease may be a Cas3 protein. In some embodiments, the Cas nuclease may be from a Type-III CRISPR/Cas system. In some embodiments, the Cas nuclease may have an RNA cleavage activity.

In some embodiments, the RNA-guided DNA-binding agent has single-strand nickase activity, i.e., can cut one DNA strand to produce a single-strand break, also known as a “nick.” In some embodiments, the RNA-guided DNA-binding agent comprises a Cas nickase. A nickase is an enzyme that creates a nick in dsDNA, i.e., cuts one strand but not the other of the DNA double helix. In some embodiments, a Cas nickase is a version of a Cas nuclease (e.g., a Cas nuclease discussed above) in which an endonucleolytic active site is inactivated, e.g., by one or more alterations (e.g., point mutations) in a catalytic domain. See, e.g., U.S. Pat. No. 8,889,356 for discussion of Cas nickases and exemplary catalytic domain alterations. In some embodiments, a Cas nickase such as a Cas9 nickase has an inactivated RuvC or HNH domain.

In some embodiments, the RNA-guided DNA-binding agent is modified to contain only one functional nuclease domain. For example, the agent protein may be modified such that one of the nuclease domains is mutated or fully or partially deleted to reduce its nucleic acid cleavage activity. In some embodiments, a nickase is used having a RuvC domain with reduced activity. In some embodiments, a nickase is used having an inactive RuvC domain. In some embodiments, a nickase is used having an HNH domain with reduced activity. In some embodiments, a nickase is used having an inactive HNH domain.

In some embodiments, a conserved amino acid within a Cas protein nuclease domain is substituted to reduce or alter nuclease activity. In some embodiments, a Cas nuclease may comprise an amino acid substitution in the RuvC or RuvC-like nuclease domain. Exemplary amino acid substitutions in the RuvC or RuvC-like nuclease domain include D10A (based on the S. pyogenes Cas9 protein). See, e.g., Zetsche et al. (2015) Cell October 22:163(3): 759-771. In some embodiments, the Cas nuclease may comprise an amino acid substitution in the HNH or HNH-like nuclease domain. Exemplary amino acid substitutions in the HNH or HNH-like nuclease domain include E762A, H840A, N863A, H983A, and D986A (based on the S. pyogenes Cas9 protein). See, e.g., Zetsche et al. (2015). Further exemplary amino acid substitutions include D917A, E1006A, and D1255A (based on the Francisella novicida U112 Cpf1 (FnCpf1) sequence (UniProtKB—A0Q7Q2 (CPF1_FRATN)).

In some embodiments, an mRNA encoding a nickase is provided in combination with a pair of guide RNAs that are complementary to the sense and antisense strands of the target sequence, respectively. In this embodiment, the guide RNAs direct the nickase to a target sequence and introduce a DSB by generating a nick on opposite strands of the target sequence (i.e., double nicking). In some embodiments, use of double nicking may improve specificity and reduce off-target effects. In some embodiments, a nickase is used together with two separate guide RNAs targeting opposite strands of DNA to produce a double nick in the target DNA. In some embodiments, a nickase is used together with two separate guide RNAs that are selected to be in close proximity to produce a double nick in the target DNA.

In some embodiments, the RNA-guided DNA-binding agent lacks cleavase and nickase activity. In some embodiments, the RNA-guided DNA-binding agent comprises a dCas DNA-binding polypeptide. A dCas polypeptide has DNA-binding activity while essentially lacking catalytic (cleavase/nickase) activity. In some embodiments, the dCas polypeptide is a dCas9 polypeptide. In some embodiments, the RNA-guided DNA-binding agent lacking cleavase and nickase activity or the dCas DNA-binding polypeptide is a version of a Cas nuclease (e.g., a Cas nuclease discussed above) in which its endonucleolytic active sites are inactivated, e.g., by one or more alterations (e.g., point mutations) in its catalytic domains. See, e.g., US 2014/0186958 A1; US 2015/0166980 A1.

In some embodiments, the RNA-guided DNA-binding agent comprises one or more heterologous functional domains (e.g., is or comprises a fusion polypeptide).

In some embodiments, the heterologous functional domain may facilitate transport of the RNA-guided DNA-binding agent into the nucleus of a cell. For example, the heterologous functional domain may be a nuclear localization signal (NLS). In some embodiments, the RNA-guided DNA-binding agent may be fused with 1-10 NLS(s). In some embodiments, the RNA-guided DNA-binding agent may be fused with 1-5 NLS(s). In some embodiments, the RNA-guided DNA-binding agent may be fused with one NLS. Where one NLS is used, the NLS may be linked at the N-terminus or the C-terminus of the RNA-guided DNA-binding agent sequence. It may also be inserted within the RNA-guided DNA binding agent sequence. In other embodiments, the RNA-guided DNA-binding agent may be fused with more than one NLS. In some embodiments, the RNA-guided DNA-binding agent may be fused with 2, 3, 4, or 5 NLSs. In some embodiments, the RNA-guided DNA-binding agent may be fused with two NLSs. In certain circumstances, the two NLSs may be the same (e.g., two SV40 NLSs) or different. In some embodiments, the RNA-guided DNA-binding agent is fused to two SV40 NLS sequences linked at the carboxy terminus. In some embodiments, the RNA-guided DNA-binding agent may be fused with two NLSs, one linked at the N-terminus and one at the C-terminus. In some embodiments, the RNA-guided DNA-binding agent may be fused with 3 NLSs. In some embodiments, the RNA-guided DNA-binding agent may be fused with no NLS. In some embodiments, the NLS may be a monopartite sequence, such as, e.g., the SV40 NLS, PKKKRKV (SEQ ID NO: 600) or PKKKRRV (SEQ ID NO: 601). In some embodiments, the NLS may be a bipartite sequence, such as the NLS of nucleoplasmin, KRPAATKKAGQAKKKK (SEQ ID NO: 602). In a specific embodiment, a single PKKKRKV (SEQ ID NO: 600) NLS may be linked at the C-terminus of the RNA-guided DNA-binding agent. One or more linkers are optionally included at the fusion site.

In some embodiments, the heterologous functional domain may be capable of modifying the intracellular half-life of the RNA-guided DNA binding agent. In some embodiments, the half-life of the RNA-guided DNA binding agent may be increased. In some embodiments, the half-life of the RNA-guided DNA-binding agent may be reduced. In some embodiments, the heterologous functional domain may be capable of increasing the stability of the RNA-guided DNA-binding agent. In some embodiments, the heterologous functional domain may be capable of reducing the stability of the RNA-guided DNA-binding agent. In some embodiments, the heterologous functional domain may act as a signal peptide for protein degradation. In some embodiments, the protein degradation may be mediated by proteolytic enzymes, such as, for example, proteasomes, lysosomal proteases, or calpain proteases. In some embodiments, the heterologous functional domain may comprise a PEST sequence. In some embodiments, the RNA-guided DNA-binding agent may be modified by addition of ubiquitin or a polyubiquitin chain. In some embodiments, the ubiquitin may be a ubiquitin-like protein (UBL). Non-limiting examples of ubiquitin-like proteins include small ubiquitin-like modifier (SUMO), ubiquitin cross-reactive protein (UCRP, also known as interferon-stimulated gene-15 (ISG15)), ubiquitin-related modifier-1 (URM1), neuronal-precursor-cell-expressed developmentally downregulated protein-8 (NEDD8, also called Rub1 in S. cerevisiae), human leukocyte antigen F-associated (FAT10), autophagy-8 (ATG8) and -12 (ATG12), Fau ubiquitin-like protein (FUB1), membrane-anchored UBL (MUB), ubiquitin fold-modifier-1 (UFM1), and ubiquitin-like protein-5 (UBL5).

In some embodiments, the heterologous functional domain may be a marker domain. Non-limiting examples of marker domains include fluorescent proteins, purification tags, epitope tags, and reporter gene sequences. In some embodiments, the marker domain may be a fluorescent protein. Non-limiting examples of suitable fluorescent proteins include green fluorescent proteins (e.g., GFP, GFP-2, tagGFP, turboGFP, sfGFP, EGFP, Emerald, Azami Green, Monomeric Azami Green, CopGFP, AceGFP, ZsGreen1), yellow fluorescent proteins (e.g., YFP, EYFP, Citrine, Venus, YPet, PhiYFP, ZsYellow1), blue fluorescent proteins (e.g., EBFP, EBFP2, Azurite, mKalamal, GFPuv, Sapphire, T-sapphire), cyan fluorescent proteins (e.g., ECFP, Cerulean, CyPet, AmCyan1, Midoriishi-Cyan), red fluorescent proteins (e.g., mKate, mKate2, mPlum, DsRed monomer, mCherry, mRFP1, DsRed-Express, DsRed2, DsRed-Monomer, HcRed-Tandem, HcRed1, AsRed2, eqFP611, mRasberry, mStrawberry, Jred), and orange fluorescent proteins (mOrange, mKO, Kusabira-Orange, Monomeric Kusabira-Orange, mTangerine, tdTomato) or any other suitable fluorescent protein. In other embodiments, the marker domain may be a purification tag and/or an epitope tag. Non-limiting exemplary tags include glutathione-S-transferase (GST), chitin binding protein (CBP), maltose binding protein (MBP), thioredoxin (TRX), poly(NANP), tandem affinity purification (TAP) tag, myc, AcV5, AU1, AU5, E, ECS, E2, FLAG, HA, nus, Softag 1, Softag 3, Strep, SBP, Glu-Glu, HSV, KT3, S, S1, T7, V5, VSV-G, 6×His, 8×His, biotin carboxyl carrier protein (BCCP), poly-His, and calmodulin. Non-limiting exemplary reporter genes include glutathione-S-transferase (GST), horseradish peroxidase (HRP), chloramphenicol acetyltransferase (CAT), beta-galactosidase, beta-glucuronidase, luciferase, or fluorescent proteins.

In additional embodiments, the heterologous functional domain may target the RNA-guided DNA-binding agent to a specific organelle, cell type, tissue, or organ. In some embodiments, the heterologous functional domain may target the RNA-guided DNA-binding agent to mitochondria.

In further embodiments, the heterologous functional domain may be an effector domain. When the RNA-guided DNA-binding agent is directed to its target sequence, e.g., when a Cas nuclease is directed to a target sequence by a gRNA, the effector domain may modify or affect the target sequence. In some embodiments, the effector domain may be chosen from a nucleic acid binding domain, a nuclease domain (e.g., a non-Cas nuclease domain), an epigenetic modification domain, a transcriptional activation domain, or a transcriptional repressor domain. In some embodiments, the heterologous functional domain is a nuclease, such as a FokI nuclease. See, e.g., U.S. Pat. No. 9,023,649. In some embodiments, the heterologous functional domain is a transcriptional activator or repressor. See, e.g., Qi et al., “Repurposing CRISPR as an RNA-guided platform for sequence-specific control of gene expression,” Cell 152:1173-83 (2013); Perez-Pinera et al., “RNA-guided gene activation by CRISPR-Cas9-based transcription factors,” Nat. Methods 10:973-6 (2013); Mali et al., “CAS9 transcriptional activators for target specificity screening and paired nickases for cooperative genome engineering,” Nat. Biotechnol. 31:833-8 (2013); Gilbert et al., “CRISPR-mediated modular RNA-guided regulation of transcription in eukaryotes,” Cell 154:442-51 (2013). As such, the RNA-guided DNA-binding agent essentially becomes a transcription factor that can be directed to bind a desired target sequence using a guide RNA.

E. Determination of Efficacy of gRNAs

In some embodiments, the efficacy of a gRNA is determined when delivered or expressed together with other components forming an RNP. In some embodiments, the gRNA is expressed together with an RNA-guided DNA binding agent, such as a Cas protein, e.g. Cas9. In some embodiments, the gRNA is delivered to or expressed in a cell line that already stably expresses an RNA-guided DNA nuclease, such as a Cas nuclease or nickase, e.g. Cas9 nuclease or nickase. In some embodiments the gRNA is delivered to a cell as part of a RNP. In some embodiments, the gRNA is delivered to a cell along with a mRNA encoding an RNA-guided DNA nuclease, such as a Cas nuclease or nickase, e.g. Cas9 nuclease or nickase.

As described herein, use of an RNA-guided DNA nuclease and a guide RNA disclosed herein can lead to double-stranded breaks in the DNA which can produce errors in the form of insertion/deletion (indel) mutations upon repair by cellular machinery. Many mutations due to indels alter the reading frame or introduce premature stop codons and, therefore, produce a non-functional protein.

In some embodiments, the efficacy of particular gRNAs is determined based on in vitro models. In some embodiments, the in vitro model is HEK293 cells stably expressing Cas9 (HEK293_Cas9). In some embodiments, the in vitro model is HUH7 human hepatocarcinoma cells. In some embodiments, the in vitro model is HepG2 cells. In some embodiments, the in vitro model is primary human hepatocytes. In some embodiments, the in vitro model is primary cynomolgus hepatocytes. With respect to using primary human hepatocytes, commercially available primary human hepatocytes can be used to provide greater consistency between experiments. In some embodiments, the number of off-target sites at which a deletion or insertion occurs in an in vitro model (e.g., in primary human hepatocytes) is determined, e.g., by analyzing genomic DNA from primary human hepatocytes transfected in vitro with Cas9 mRNA and the guide RNA. In some embodiments, such a determination comprises analyzing genomic DNA from primary human hepatocytes transfected in vitro with Cas9 mRNA, the guide RNA, and a donor oligonucleotide. Exemplary procedures for such determinations are provided in the working examples below.

In some embodiments, the efficacy of particular gRNAs is determined across multiple in vitro cell models for a gRNA selection process. In some embodiments, a cell line comparison of data with selected gRNAs is performed. In some embodiments, cross screening in multiple cell models is performed.

In some embodiments, the efficacy of particular gRNAs is determined based on in vivo models. In some embodiments, the in vivo model is a rodent model. In some embodiments, the rodent model is a mouse which expresses a Hao1 gene. In some embodiments, the rodent model is a mouse which expresses a human HAO1 gene. In some embodiments, the in vivo model is a non-human primate, for example cynomolgus monkey.

In some embodiments, the efficacy of a guide RNA is measured by percent editing of HAO1. In some embodiments, the percent editing of HAO1 is compared to the percent editing necessary to achieve knockdown of HAO1 protein, e.g., from whole cell lysates in the case of an in vitro model or in tissue in the case of an in vivo model.

In some embodiments, the efficacy of a guide RNA is measured by the number and/or frequency of indels at off-target sequences within the genome of the target cell type. In some embodiments, efficacious guide RNAs are provided which produce indels at off target sites at very low frequencies (e.g., <5%) in a cell population and/or relative to the frequency of indel creation at the target site. Thus, the disclosure provides for guide RNAs which do not exhibit off-target indel formation in the target cell type (e.g., a hepatocyte), or which produce a frequency of off-target indel formation of <5% in a cell population and/or relative to the frequency of indel creation at the target site. In some embodiments, the disclosure provides guide RNAs which do not exhibit any off target indel formation in the target cell type (e.g., hepatocyte). In some embodiments, guide RNAs are provided which produce indels at less than 5 off-target sites, e.g., as evaluated by one or more methods described herein. In some embodiments, guide RNAs are provided which produce indels at less than or equal to 4, 3, 2, or 1 off-target site(s) e.g., as evaluated by one or more methods described herein. In some embodiments, the off-target site(s) does not occur in a protein coding region in the target cell (e.g., hepatocyte) genome.

In some embodiments, detecting gene editing events, such as the formation of insertion/deletion (“indel”) mutations and homology directed repair (HDR) events in target DNA utilize linear amplification with a tagged primer and isolating the tagged amplification products (herein after referred to as “LAM-PCR,” or “Linear Amplification (LA)” method).

In some embodiments, the efficacy of a guide RNA is measured by mearing levels of glycolate and/or levels of oxalate in a sample such as a body fluid, e.g., serum, plasma, blood, or urine. In some embodiments, the efficacy of a guide RNA is measured by mearing levels of glycolate in the serum or plasma and/or levels of oxalate in the urine. An increase in the levels of glycolate in the serum or plasma and/or a decrease in the level of oxalate in the urine is indicative of an effective guide RNA. In some embodiments, urinary oxalate is reduced below 0.7 mmol/24 hrs/1.73 m². In some embodiments, levels of glycolate and oxalate are measured using an enzyme-linked immunosorbent assay (ELISA) assay with cell culture media or serum or plasma. In some embodiments, levels of glycolate and oxalate are measured in the same in vitro or in vivo systems or models used to measure editing. In some embodiments, levels of glycolate and oxalate are measured in cells, e.g., primary human hepatocytes. In some embodiments, levels of glycolate and oxalate are measured in HUH7 cells. In some embodiments, levels of glycolate and oxalate are measured in HepG2 cells.

### III. Therapeutic Methods

The gRNAs and associated methods and compositions disclosed herein are useful in treating and preventing PH1 and preventing symptoms of PH1. In some embodiments, the gRNAs disclosed herein are useful in treating and preventing calcium oxalate production, calcium oxalate deposition in organs, hyperoxaluria, oxalosis, including systemic oxalosis, and hematuria. In some embodiments, the gRNAs disclosed herein are useful in delaying or emeliorating the need for kidney or liver transplant. In some embodiments, the gRNAs disclosed herein are useful in preventing end stage renal disease (ESRD). Administration of the gRNAs disclosed herein will increase serum or plasma glycolate and decrease oxalate production or accumulation so that less oxalate is excreted in the urine. Therefore, in one aspect, effectiveness of treatment/prevention can be assessed by measuring serum or plasma glycolate, wherein an increase in glycolate levels indicates effectiveness. In some embodiments, effectiveness of treatment/prevention can be assessed by measuring oxalate in a sample, such as urinary oxalate, wherein a decrease in urinary oxalate indicates effectiveness.

Normal daily oxalate excretion in the urine of healthy subjects ranges between 10-40 mg per 24 hours, while concentrations exceeding 40-45 mg per 24 hours are considered to be clinical hyperoxaluria (See e.g., Bhasin et al., World J Nephrol 2015 May 6; 4(2): 235-244). Accordingly, in some embodiments, administration of the gRNAs and compositions disclosed herein are useful for reducing levels of oxalate such that a subject no longer exhibits levels of urinary oxalate associated with clinical hyperoxaluria. In some embodiments, administration of the gRNAs and compositions disclosed herein reduces a subject's urinary oxalate to less than 40 mg in a 24 hour period. In some embodiments, administration of the gRNAs and compositions disclosed herein reduces a subject's urinary oxalate to less than 35, less than 30, less than 25, less than 20, less than 15, or less than 10 mg in a 24 hour period.

In some embodiments, any one or more of the gRNAs, compositions, or pharmaceutical formulations described herein is for use in preparing a medicament for treating or preventing a disease or disorder in a subject. In some embodiments, treatment and/or prevention is accomplished with a single dose, e.g., one-time treatment, of medicament/composition. In some embodiments, the disease or disorder is PH1.

In some embodiments, the invention comprises a method of treating or preventing a disease or disorder in subject comprising administering any one or more of the gRNAs, compositions, or pharmaceutical formulations described herein. In some embodiments, the disease or disorder is PH1. In some embodiments, the gRNAs, compositions, or pharmaceutical formulations described herein are administered as a single dose, e.g., at one time. In some embodiments, the single dose achieves durable treatment and/or prevention. In some embodiments, the method achieves durable treatment and/or prevention. Durable treatment and/or prevention, as used herein, includes treatment and/or prevention that extends at least i) 3, 4, 5, 6, 7, 8, 9, 10, 11, 12, 13, 14, or 15 weeks; ii) 1, 2, 3, 4, 5, 6, 7, 8, 9, 10, 11, 12, 18, 24, 30, or 36 months; or iii) 1, 2, 3, 4, 5, 6, 7, 8, 9, or 10 years. In some embodiments, a single dose of the gRNAs, compositions, or pharmaceutical formulations described herein is sufficient to treat and/or prevent any of the indications described herein for the duration of the subject's life.

In some embodiments, the invention comprises a method or use of modifying (e.g., creating a double strand break) a target DNA comprising, administering or delivering any one or more of the gRNAs, compositions, or pharmaceutical formulations described herein. In some embodiments, the target DNA is the HAO1 gene. In some embodiments, the target DNA is in an exon of the HAO1 gene. In some embodiments, the target DNA is in exon 1, 2, 3, 4, 5, 6, 7, or 8 of the HAO1 gene.

In some embodiments, the invention comprises a method or use for modulation of a target gene comprising, administering or delivering any one or more of the gRNAs, compositions, or pharmaceutical formulations described herein. In some embodiments, the modulation is editing of the HAO1 target gene. In some embodiments, the modulation is a change in expression of the protein encoded by the HAO1 target gene.

In some embodiments, the method or use results in gene editing. In some embodiments, the method or use results in a double-stranded break within the target HAO1 gene. In some embodiments, the method or use results in formation of indel mutations during non-homologous end joining of the DSB. In some embodiments, the method or use results in an insertion or deletion of nucleotides in a target HAO1 gene. In some embodiments, the insertion or deletion of nucleotides in a target HAO1 gene leads to a frameshift mutation or premature stop codon that results in a non-functional protein. In some embodiments, the insertion or deletion of nucleotides in a target HAO1 gene leads to a knockdown or elimination of target gene expression. In some embodiments, the method or use comprises homology directed repair of a DSB.

In some embodiments, the method or use results in HAO1 gene modulation. In some embodiments, the HAO1 gene modulation is a decrease in gene expression. In some embodiments, the method or use results in decreased expression of the protein encoded by the target gene.

In some embodiments, a method of inducing a double-stranded break (DSB) within the HAO1 gene is provided comprising administering a composition comprising a guide RNA comprising any one or more guide sequences of SEQ ID NOs:1-146, or any one or more of the sgRNAs of SEQ ID Nos: 151-168 or 251-268. In some embodiments, gRNAs comprising any one or more of the guide sequences of SEQ ID NOs:1-146 are administered to induce a DSB in the HAO1 gene. The guide RNAs may be administered together with an RNA-guided DNA nuclease such as a Cas nuclease (e.g., Cas9) or an mRNA or vector encoding an RNA-guided DNA nuclease such as a Cas nuclease (e.g., Cas9).

In some embodiments, a method of modifying the HAO1 gene is provided comprising administering a composition comprising a guide RNA comprising any one or more of the guide sequences of SEQ ID NOs:1-146, or any one or more of the sgRNAs of SEQ ID Nos: 151-168 or 251-268. In some embodiments, gRNAs comprising any one or more of the guide sequences of SEQ ID NOs:1-146, or any one or more of the sgRNAs of SEQ ID Nos: 151-168 or 251-268, are administered to modify the HAO1 gene. The guide RNAs may be administered together with an RNA-guided DNA nuclease such as a Cas nuclease (e.g., Cas9) or an mRNA or vector encoding an RNA-guided DNA nuclease such as a Cas nuclease (e.g., Cas9).

In some embodiments, a method of treating or preventing PH1 is provided comprising administering a composition comprising a guide RNA comprising any one or more of the guide sequences of SEQ ID NOs:1-146, or any one or more of the sgRNAs of SEQ ID Nos: 151-168 or 251-268. In some embodiments, gRNAs comprising any one or more of the guide sequences of SEQ ID NOs:1-146, or any one or more of the sgRNAs of SEQ ID Nos: 151-168 or 251-268 are administered to treat or prevent PH1. The guide RNAs may be administered together with an RNA-guided DNA nuclease such as a Cas nuclease (e.g., Cas9) or an mRNA or vector encoding an RNA-guided DNA nuclease such as a Cas nuclease (e.g., Cas9).

In some embodiments, a method of decreasing or eliminating calcium oxalate production and/or deposition is provided comprising administering a guide RNA comprising any one or more of the guide sequences of SEQ ID NOs:1-146, or any one or more of the sgRNAs of SEQ ID Nos: 151-168 or 251-268. The guide RNAs may be administered together with an RNA-guided DNA nuclease such as a Cas nuclease (e.g., Cas9) or an mRNA or vector encoding an RNA-guided DNA nuclease such as a Cas nuclease (e.g., Cas9).

In some embodiments, a method of treating or preventing hyperoxaluria is provided comprising administering a guide RNA comprising any one or more of the guide sequences of SEQ ID NOs:1-146, or any one or more of the sgRNAs of SEQ ID Nos: 151-168 or 251-268. The guide RNAs may be administered together with an RNA-guided DNA nuclease such as a Cas nuclease (e.g., Cas9) or an mRNA or vector encoding an RNA-guided DNA nuclease such as a Cas nuclease (e.g., Cas9).

In some embodiments, a method of treating or preventing oxalosis, including systemic oxalosis is provided comprising administering a guide RNA comprising any one or more of the guide sequences of SEQ ID NOs:1-146, or any one or more of the sgRNAs of SEQ ID Nos: 151-168 or 251-268. The guide RNAs may be administered together with an RNA-guided DNA nuclease such as a Cas nuclease (e.g., Cas9) or an mRNA or vector encoding an RNA-guided DNA nuclease such as a Cas nuclease (e.g., Cas9).

In some embodiments, a method of treating or preventing hematuria is provided comprising administering a guide RNA comprising any one or more of the guide sequences of SEQ ID NOs:1-146, or any one or more of the sgRNAs of SEQ ID Nos: 151-168 or 251-268. The guide RNAs may be administered together with an RNA-guided DNA nuclease such as a Cas nuclease (e.g., Cas9) or an mRNA or vector encoding an RNA-guided DNA nuclease such as a Cas nuclease (e.g., Cas9).

In some embodiments, gRNAs comprising any one or more of the guide sequences of SEQ ID NOs:1-146 or any one or more of the sgRNAs of SEQ ID Nos: 151-168 or 251-268 are administered to reduce oxalate levels in the urine. The gRNAs may be administered together with an RNA-guided DNA nuclease such as a Cas nuclease (e.g., Cas9) or an mRNA or vector encoding an RNA-guided DNA nuclease such as a Cas nuclease (e.g., Cas9).

In some embodiments, gRNAs comprising any one or more of the guide sequences of SEQ ID NOs:1-146 or any one or more of the sgRNAs of SEQ ID Nos: 151-168 or 251-268 are administered to increase serum glycolate in the serum or plasma. The gRNAs may be administered together with an RNA-guided DNA nuclease such as a Cas nuclease (e.g., Cas9) or an mRNA or vector encoding an RNA-guided DNA nuclease such as a Cas nuclease (e.g., Cas9).

In some embodiments, the gRNAs comprising the guide sequences of Table 1 together with an RNA-guided DNA nuclease such as a Cas nuclease induce DSBs, and non-homologous ending joining (NHEJ) during repair leads to a mutation in the HAO1 gene. In some embodiments, NHEJ leads to a deletion or insertion of a nucleotide(s), which induces a frame shift or nonsense mutation in the HAO1 gene.

In some embodiments, administering the guide RNAs of the invention (e.g., in a composition provided herein) increases levels (e.g., serum or plasma levels) of glycolate in the subject, and therefore prevents oxalate accumulation.

In some embodiments, increasing serum glycolate results in a decrease of urinary oxalate. In some embodiments, reduction of urinary oxalate reduces or eliminate calcium oxalate formation and deposition in organs.

In some embodiments, the subject is mammalian. In some embodiments, the subject is human. In some embodiments, the subject is cow, pig, monkey, sheep, dog, cat, fish, or poultry.

In some embodiments, the use of a guide RNAs comprising any one or more of the guide sequences in Table 1 or one or more sgRNAs from Table 2 (e.g., in a composition provided herein) is provided for the preparation of a medicament for treating a human subject having PH1.

In some embodiments, the guide RNAs, compositions, and formulations are administered intravenously. In some embodiments, the guide RNAs, compositions, and formulations are administered into the hepatic circulation.

In some embodiments, a single administration of a composition comprising a guide RNA provided herein is sufficient to knock down expression of the mutant protein. In other embodiments, more than one administration of a composition comprising a guide RNA provided herein may be beneficial to maximize therapeutic effects.

In some embodiments, treatment slows or halts PH1 disease progression.

In some embodiments, treatment slows or halts progression of end stage renal disease (ESRD). In some embodiments, treatment slows or halts the need for kidney and/or liver transplant. In some embodiments, treatment results in improvement, stabilization, or slowing of change in symptoms of PH1.

### A. Combination Therapy

In some embodiments, the invention comprises combination therapies comprising any one of the gRNAs comprising any one or more of the guide sequences disclosed in Table 1 (e.g., in a composition provided herein) together with an additional therapy suitable for alleviating PH1 and its symptoms, as described above.

In some embodiments, the additional therapy for PH1 is vitamin B6, hydration, renal dialysis, or liver or kidney transplant. In some embodiments, the additional therapy is lumasiran (ALN-GO1; Alnylam).

In some embodiments, the combination therapy comprises any one of the gRNAs comprising any one or more of the guide sequences disclosed in Table 1 together with a siRNA that targets HAO1. In some embodiments, the siRNA is any siRNA capable of further reducing or eliminating the expression of wild type or mutant HAO1. In some embodiments, the siRNA is the drug lumasiran (ALN-GO1; Alnylam). In some embodiments, the siRNA is administered after any one of the gRNAs comprising any one or more of the guide sequences disclosed in Table 1 (e.g., in a composition provided herein). In some embodiments, the siRNA is administered on a regular basis following treatment with any of the gRNA compositions provided herein.

In some embodiments, the combination therapy comprises any one of the gRNAs comprising any one or more of the guide sequences disclosed in Table 1 (e.g., in a composition provided herein) together with antisense nucleotide that targets HAO1. In some embodiments, the antisense nucleotide is any antisense nucleotide capable of further reducing or eliminating the expression of HAO1. In some embodiments, the antisense nucleotide is administered after any one of the gRNAs comprising any one or more of the guide sequences disclosed in Table 1 (e.g., in a composition provided herein). In some embodiments, the antisense nucleotide is administered on a regular basis following treatment with any of the gRNA compositions provided herein.

B. Delivery of gRNA Compositions

Lipid nanoparticles (LNPs) are a well-known means for delivery of nucleotide and protein cargo, and may be used for delivery of the guide RNAs, compositions, or pharmaceutical formulations disclosed herein. In some embodiments, the LNPs deliver nucleic acid, protein, or nucleic acid together with protein.

In some embodiments, the invention comprises a method for delivering any one of the gRNAs disclosed herein to a subject, wherein the gRNA is associated with an LNP. In some embodiments, the gRNA/LNP is also associated with a Cas9 or an mRNA encoding Cas9.

In some embodiments, the invention comprises a composition comprising any one of the gRNAs disclosed and an LNP. In some embodiments, the composition further comprises a Cas9 or an mRNA encoding Cas9.

In some embodiments, the LNPs comprise cationic lipids. In some embodiments, the LNPs comprise (9Z,12Z)-3-((4,4-bis(octyloxy)butanoyl)oxy)-2-((((3-(diethylamino)propoxy)carbonyl)oxy)methyl)propyl octadeca-9,12-dienoate, also called 3-((4,4-bis(octyloxy)butanoyl)oxy)-2-((((3-(diethylamino)propoxy)carbonyl)oxy)methyl)propyl (9Z,12Z)-octadeca-9,12-dienoate) or another ionizable lipid. See, e.g., lipids of WO/2017/173054 and references described therein. In some embodiments, the LNPs comprise molar ratios of a cationic lipid amine to RNA phosphate (N:P) of about 4.5, 5.0, 5.5, 6.0, or 6.5. In some embodiments, the term cationic and ionizable in the context of LNP lipids is interchangeable, e.g., wherein ionizable lipids are cationic depending on the pH.

In some embodiments, LNPs associated with the gRNAs disclosed herein are for use in preparing a medicament for treating a disease or disorder.

Electroporation is a well-known means for delivery of cargo, and any electroporation methodology may be used for delivery of any one of the gRNAs disclosed herein. In some embodiments, electroporation may be used to deliver any one of the gRNAs disclosed herein and Cas9 or an mRNA encoding Cas9.

In some embodiments, the invention comprises a method for delivering any one of the gRNAs disclosed herein to an ex vivo cell, wherein the gRNA is associated with an LNP or not associated with an LNP. In some embodiments, the gRNA/LNP or gRNA is also associated with a Cas9 or an mRNA encoding Cas9.

In some embodiments, the guide RNA compositions described herein, alone or encoded on one or more vectors, are formulated in or administered via a lipid nanoparticle; see e.g., WO/2017/173054, filed Mar. 30, 2017 and published May 10, 2017 entitled “LIPID NANOPARTICLE FORMULATIONS FOR CRISPR/CAS COMPONENTS,” the contents of which are hereby incorporated by reference in their entirety.

In certain embodiments, the invention comprises DNA or RNA vectors encoding any of the guide RNAs comprising any one or more of the guide sequences described herein. In some embodiments, in addition to guide RNA sequences, the vectors further comprise nucleic acids that do not encode guide RNAs. Nucleic acids that do not encode guide RNA include, but are not limited to, promoters, enhancers, regulatory sequences, and nucleic acids encoding an RNA-guided DNA nuclease, which can be a nuclease such as Cas9. In some embodiments, the vector comprises one or more nucleotide sequence(s) encoding a crRNA, a trRNA, or a crRNA and trRNA. In some embodiments, the vector comprises one or more nucleotide sequence(s) encoding a sgRNA and an mRNA encoding an RNA-guided DNA nuclease, which can be a Cas nuclease, such as Cas9 or Cpf1. In some embodiments, the vector comprises one or more nucleotide sequence(s) encoding a crRNA, a trRNA, and an mRNA encoding an RNA-guided DNA nuclease, which can be a Cas protein, such as, Cas9. In one embodiment, the Cas9 is from Streptococcus pyogenes (i.e., Spy Cas9). In some embodiments, the nucleotide sequence encoding the crRNA, trRNA, or crRNA and trRNA (which may be a sgRNA) comprises or consists of a guide sequence flanked by all or a portion of a repeat sequence from a naturally-occurring CRISPR/Cas system. The nucleic acid comprising or consisting of the crRNA, trRNA, or crRNA and trRNA may further comprise a vector sequence wherein the vector sequence comprises or consists of nucleic acids that are not naturally found together with the crRNA, trRNA, or crRNA and trRNA.

This description and exemplary embodiments should not be taken as limiting. For the purposes of this specification and appended claims, unless otherwise indicated, all numbers expressing quantities, percentages, or proportions, and other numerical values used in the specification and claims, are to be understood as being modified in all instances by the term “about,” to the extent they are not already so modified. Accordingly, unless indicated to the contrary, the numerical parameters set forth in the following specification and attached claims are approximations that may vary depending upon the desired properties sought to be obtained. At the very least, and not as an attempt to limit the application of the doctrine of equivalents to the scope of the claims, each numerical parameter should at least be construed in light of the number of reported significant digits and by applying ordinary rounding techniques.

It is noted that, as used in this specification and the appended claims, the singular forms “a,” “an,” and “the,” and any singular use of any word, include plural referents unless expressly and unequivocally limited to one referent. As used herein, the term “include” and its grammatical variants are intended to be non-limiting, such that recitation of items in a list is not to the exclusion of other like items that can be substituted or added to the listed items.

### EXAMPLES

The following examples are provided to illustrate certain disclosed embodiments and are not to be construed as limiting the scope of this disclosure in any way.

### Example 1—Materials and Methods

In Vitro Transcription (“IVT”) of Nuclease mRNA

Capped and polyadenylated Streptococcus pyogenes (“Spy”) Cas9 mRNA containing N1-methyl pseudo-U was generated by in vitro transcription using a linearized plasmid DNA template and T7 RNA polymerase. Plasmid DNA containing a T7 promoter and a 100 nt poly (A/T) region was linearized by incubating at 37° C. for 2 hours with XbaI with the following conditions: 200 ng/μL plasmid, 2 U/μL XbaI (NEB), and 1× reaction buffer. The XbaI was inactivated by heating the reaction at 65° C. for 20 min. The linearized plasmid was purified from enzyme and buffer salts using a silica maxi spin column (Epoch Life Sciences) and analyzed by agarose gel to confirm linearization. The IVT reaction to generate Cas9 modified mRNA was incubated at 37° C. for 4 hours in the following conditions: 50 ng/μL linearized plasmid; 2 mM each of GTP, ATP, CTP, and N1-methyl pseudo-UTP (Trilink); 10 mM ARCA (Trilink); 5 U/μL T7 RNA polymerase (NEB); 1 U/μL Murine RNase inhibitor (NEB); 0.004 U/μL Inorganic E. coli pyrophosphatase (NEB); and 1× reaction buffer. After the 4-hour incubation, TURBO DNase (ThermoFisher) was added to a final concentration of 0.01 U/μL, and the reaction was incubated for an additional 30 minutes to remove the DNA template. The Cas9 mRNA was purified from enzyme and nucleotides using a MegaClear Transcription Clean-up kit according to the manufacturer's protocol (ThermoFisher). Alternatively, the Cas9 mRNA was purified with a LiCl precipitation method, which in some cases was followed by further purification by tangential flow filtration. The transcript concentration was determined by measuring the light absorbance at 260 nm (Nanodrop), and the transcript was analyzed by capillary electrophoresis by Bioanalyzer (Agilent).

The sequences for transcription of Cas9 mRNA used in the Examples comprised either SEQ ID NO: 500 or SEQ ID NO: 501.

SEQ ID NO: 500:
ATGGATAAGAAGTACTCAATCGGGCTGGATATCGGAACTAATTCCGTGGGTTGGGCAGTGATCACGGATGAATAC
AAAGTGCCGTCCAAGAAGTTCAAGGTCCTGGGGAACACCGATAGACACAGCATCAAGAAAAATCTCATCGGAGCC
CTGCTGTTTGACTCCGGCGAAACCGCAGAAGCGACCCGGCTCAAACGTACCGCGAGGCGACGCTACACCCGGCGG
AAGAATCGCATCTGCTATCTGCAAGAGATCTTTTCGAACGAAATGGCAAAGGTCGACGACAGCTTCTTCCACCGC
CTGGAAGAATCTTTCCTGGTGGAGGAGGACAAGAAGCATGAACGGCATCCTATCTTTGGAAACATCGTCGACGAA
GTGGCGTACCACGAAAAGTACCCGACCATCTACCATCTGCGGAAGAAGTTGGTTGACTCAACTGACAAGGCCGAC
CTCAGATTGATCTACTTGGCCCTCGCCCATATGATCAAATTCCGCGGACACTTCCTGATCGAAGGCGATCTGAAC
CCTGATAACTCCGACGTGGATAAGCTTTTCATTCAACTGGTGCAGACCTACAACCAACTGTTCGAAGAAAACCCA
ATCAATGCTAGCGGCGTCGATGCCAAGGCCATCCTGTCCGCCCGGCTGTCGAAGTCGCGGCGCCTCGAAAACCTG
ATCGCACAGCTGCCGGGAGAGAAAAAGAACGGACTTTTCGGCAACTTGATCGCTCTCTCACTGGGACTCACTCCC
AATTTCAAGTCCAATTTTGACCTGGCCGAGGACGCGAAGCTGCAACTCTCAAAGGACACCTACGACGACGACTTG
GACAATTTGCTGGCACAAATTGGCGATCAGTACGCGGATCTGTTCCTTGCCGCTAAGAACCTTTCGGACGCAATC
TTGCTGTCCGATATCCTGCGCGTGAACACCGAAATAACCAAAGCGCCGCTTAGCGCCTCGATGATTAAGCGGTAC
GACGAGCATCACCAGGATCTCACGCTGCTCAAAGCGCTCGTGAGACAGCAACTGCCTGAAAAGTACAAGGAGATC
TTCTTCGACCAGTCCAAGAATGGGTACGCAGGGTACATCGATGGAGGCGCTAGCCAGGAAGAGTTCTATAAGTTC
ATCAAGCCAATCCTGGAAAAGATGGACGGAACCGAAGAACTGCTGGTCAAGCTGAACAGGGAGGATCTGCTCCGG
AAACAGAGAACCTTTGACAACGGATCCATTCCCCACCAGATCCATCTGGGTGAGCTGCACGCCATCTTGCGGCGC
CAGGAGGACTTTTACCCATTCCTCAAGGACAACCGGGAAAAGATCGAGAAAATTCTGACGTTCCGCATCCCGTAT
TACGTGGGCCCACTGGCGCGCGGCAATTCGCGCTTCGCGTGGATGACTAGAAAATCAGAGGAAACCATCACTCCT
TGGAATTTCGAGGAAGTTGTGGATAAGGGAGCTTCGGCACAAAGCTTCATCGAACGAATGACCAACTTCGACAAG
AATCTCCCAAACGAGAAGGTGCTTCCTAAGCACAGCCTCCTTTACGAATACTTCACTGTCTACAACGAACTGACT
AAAGTGAAATACGTTACTGAAGGAATGAGGAAGCCGGCCTTTCTGTCCGGAGAACAGAAGAAAGCAATTGTCGAT
CTGCTGTTCAAGACCAACCGCAAGGTGACCGTCAAGCAGCTTAAAGAGGACTACTTCAAGAAGATCGAGTGTTTC
GACTCAGTGGAAATCAGCGGGGTGGAGGACAGATTCAACGCTTCGCTGGGAACCTATCATGATCTCCTGAAGATC
ATCAAGGACAAGGACTTCCTTGACAACGAGGAGAACGAGGACATCCTGGAAGATATCGTCCTGACCTTGACCCTT
TTCGAGGATCGCGAGATGATCGAGGAGAGGCTTAAGACCTACGCTCATCTCTTCGACGATAAGGTCATGAAACAA
CTCAAGCGCCGCCGGTACACTGGTTGGGGCCGCCTCTCCCGCAAGCTGATCAACGGTATTCGCGATAAACAGAGC
GGTAAAACTATCCTGGATTTCCTCAAATCGGATGGCTTCGCTAATCGTAACTTCATGCAATTGATCCACGACGAC
AGCCTGACCTTTAAGGAGGACATCCAAAAAGCACAAGTGTCCGGACAGGGAGACTCACTCCATGAACACATCGCG
AATCTGGCCGGTTCGCCGGCGATTAAGAAGGGAATTCTGCAAACTGTGAAGGTGGTCGACGAGCTGGTGAAGGTC
ATGGGACGGCACAAACCGGAGAATATCGTGATTGAAATGGCCCGAGAAAACCAGACTACCCAGAAGGGCCAGAAA
AACTCCCGCGAAAGGATGAAGCGGATCGAAGAAGGAATCAAGGAGCTGGGCAGCCAGATCCTGAAAGAGCACCCG
GTGGAAAACACGCAGCTGCAGAACGAGAAGCTCTACCTGTACTATTTGCAAAATGGACGGGACATGTACGTGGAC
CAAGAGCTGGACATCAATCGGTTGTCTGATTACGACGTGGACCACATCGTTCCACAGTCCTTTCTGAAGGATGAC
TCGATCGATAACAAGGTGTTGACTCGCAGCGACAAGAACAGAGGGAAGTCAGATAATGTGCCATCGGAGGAGGTC
GTGAAGAAGATGAAGAATTACTGGCGGCAGCTCCTGAATGCGAAGCTGATTACCCAGAGAAAGTTTGACAATCTC
ACTAAAGCCGAGCGCGGCGGACTCTCAGAGCTGGATAAGGCTGGATTCATCAAACGGCAGCTGGTCGAGACTCGG
CAGATTACCAAGCACGTGGCGCAGATCTTGGACTCCCGCATGAACACTAAATACGACGAGAACGATAAGCTCATC
CGGGAAGTGAAGGTGATTACCCTGAAAAGCAAACTTGTGTCGGACTTTCGGAAGGACTTTCAGTTTTACAAAGTG
AGAGAAATCAACAACTACCATCACGCGCATGACGCATACCTCAACGCTGTGGTCGGTACCGCCCTGATCAAAAAG
TACCCTAAACTTGAATCGGAGTTTGTGTACGGAGACTACAAGGTCTACGACGTGAGGAAGATGATAGCCAAGTCC
GAACAGGAAATCGGGAAAGCAACTGCGAAATACTTCTTTTACTCAAACATCATGAACTTTTTCAAGACTGAAATT
ACGCTGGCCAATGGAGAAATCAGGAAGAGGCCACTGATCGAAACTAACGGAGAAACGGGCGAAATCGTGTGGGAC
AAGGGCAGGGACTTCGCAACTGTTCGCAAAGTGCTCTCTATGCCGCAAGTCAATATTGTGAAGAAAACCGAAGTG
CAAACCGGCGGATTTTCAAAGGAATCGATCCTCCCAAAGAGAAATAGCGACAAGCTCATTGCACGCAAGAAAGAC
TGGGACCCGAAGAAGTACGGAGGATTCGATTCGCCGACTGTCGCATACTCCGTCCTCGTGGTGGCCAAGGTGGAG
AAGGGAAAGAGCAAAAAGCTCAAATCCGTCAAAGAGCTGCTGGGGATTACCATCATGGAACGATCCTCGTTCGAG
AAGAACCCGATTGATTTCCTCGAGGCGAAGGGTTACAAGGAGGTGAAGAAGGATCTGATCATCAAACTCCCCAAG
TACTCACTGTTCGAACTGGAAAATGGTCGGAAGCGCATGCTGGCTTCGGCCGGAGAACTCCAAAAAGGAAATGAG
CTGGCCTTGCCTAGCAAGTACGTCAACTTCCTCTATCTTGCTTCGCACTACGAAAAACTCAAAGGGTCACCGGAA
GATAACGAACAGAAGCAGCTTTTCGTGGAGCAGCACAAGCATTATCTGGATGAAATCATCGAACAAATCTCCGAG
TTTTCAAAGCGCGTGATCCTCGCCGACGCCAACCTCGACAAAGTCCTGTCGGCCTACAATAAGCATAGAGATAAG
CCGATCAGAGAACAGGCCGAGAACATTATCCACTTGTTCACCCTGACTAACCTGGGAGCCCCAGCCGCCTTCAAG
TACTTCGATACTACTATCGATCGCAAAAGATACACGTCCACCAAGGAAGTTCTGGACGCGACCCTGATCCACCAA
AGCATCACTGGACTCTACGAAACTAGGATCGATCTGTCGCAGCTGGGTGGCGAT
SEQ ID NO: 501:
GGGTCCCGCAGTCGGCGTCCAGCGGCTCTGCTTGTTCGTGTGTGTGTCGTTGCAGGCCTTATTCGGATCCGCCAC
CATGGACAAGAAGTACAGCATCGGACTGGACATCGGAACAAACAGCGTCGGATGGGCAGTCATCACAGACGAATA
CAAGGTCCCGAGCAAGAAGTTCAAGGTCCTGGGAAACACAGACAGACACAGCATCAAGAAGAACCTGATCGGAGC
ACTGCTGTTCGACAGCGGAGAAACAGCAGAAGCAACAAGACTGAAGAGAACAGCAAGAAGAAGATACACAAGAAG
AAAGAACAGAATCTGCTACCTGCAGGAAATCTTCAGCAACGAAATGGCAAAGGTCGACGACAGCTTCTTCCACAG
ACTGGAAGAAAGCTTCCTGGTCGAAGAAGACAAGAAGCACGAAAGACACCCGATCTTCGGAAACATCGTCGACGA
AGTCGCATACCACGAAAAGTACCCGACAATCTACCACCTGAGAAAGAAGCTGGTCGACAGCACAGACAAGGCAGA
CCTGAGACTGATCTACCTGGCACTGGCACACATGATCAAGTTCAGAGGACACTTCCTGATCGAAGGAGACCTGAA
CCCGGACAACAGCGACGTCGACAAGCTGTTCATCCAGCTGGTCCAGACATACAACCAGCTGTTCGAAGAAAACCC
GATCAACGCAAGCGGAGTCGACGCAAAGGCAATCCTGAGCGCAAGACTGAGCAAGAGCAGAAGACTGGAAAACCT
GATCGCACAGCTGCCGGGAGAAAAGAAGAACGGACTGTTCGGAAACCTGATCGCACTGAGCCTGGGACTGACACC
GAACTTCAAGAGCAACTTCGACCTGGCAGAAGACGCAAAGCTGCAGCTGAGCAAGGACACATACGACGACGACCT
GGACAACCTGCTGGCACAGATCGGAGACCAGTACGCAGACCTGTTCCTGGCAGCAAAGAACCTGAGCGACGCAAT
CCTGCTGAGCGACATCCTGAGAGTCAACACAGAAATCACAAAGGCACCGCTGAGCGCAAGCATGATCAAGAGATA
CGACGAACACCACCAGGACCTGACACTGCTGAAGGCACTGGTCAGACAGCAGCTGCCGGAAAAGTACAAGGAAAT
CTTCTTCGACCAGAGCAAGAACGGATACGCAGGATACATCGACGGAGGAGCAAGCCAGGAAGAATTCTACAAGTT
CATCAAGCCGATCCTGGAAAAGATGGACGGAACAGAAGAACTGCTGGTCAAGCTGAACAGAGAAGACCTGCTGAG
AAAGCAGAGAACATTCGACAACGGAAGCATCCCGCACCAGATCCACCTGGGAGAACTGCACGCAATCCTGAGAAG
ACAGGAAGACTTCTACCCGTTCCTGAAGGACAACAGAGAAAAGATCGAAAAGATCCTGACATTCAGAATCCCGTA
CTACGTCGGACCGCTGGCAAGAGGAAACAGCAGATTCGCATGGATGACAAGAAAGAGCGAAGAAACAATCACACC
GTGGAACTTCGAAGAAGTCGTCGACAAGGGAGCAAGCGCACAGAGCTTCATCGAAAGAATGACAAACTTCGACAA
GAACCTGCCGAACGAAAAGGTCCTGCCGAAGCACAGCCTGCTGTACGAATACTTCACAGTCTACAACGAACTGAC
AAAGGTCAAGTACGTCACAGAAGGAATGAGAAAGCCGGCATTCCTGAGCGGAGAACAGAAGAAGGCAATCGTCGA
CCTGCTGTTCAAGACAAACAGAAAGGTCACAGTCAAGCAGCTGAAGGAAGACTACTTCAAGAAGATCGAATGCTT
CGACAGCGTCGAAATCAGCGGAGTCGAAGACAGATTCAACGCAAGCCTGGGAACATACCACGACCTGCTGAAGAT
CATCAAGGACAAGGACTTCCTGGACAACGAAGAAAACGAAGACATCCTGGAAGACATCGTCCTGACACTGACACT
GTTCGAAGACAGAGAAATGATCGAAGAAAGACTGAAGACATACGCACACCTGTTCGACGACAAGGTCATGAAGCA
GCTGAAGAGAAGAAGATACACAGGATGGGGAAGACTGAGCAGAAAGCTGATCAACGGAATCAGAGACAAGCAGAG
CGGAAAGACAATCCTGGACTTCCTGAAGAGCGACGGATTCGCAAACAGAAACTTCATGCAGCTGATCCACGACGA
CAGCCTGACATTCAAGGAAGACATCCAGAAGGCACAGGTCAGCGGACAGGGAGACAGCCTGCACGAACACATCGC
AAACCTGGCAGGAAGCCCGGCAATCAAGAAGGGAATCCTGCAGACAGTCAAGGTCGTCGACGAACTGGTCAAGGT
CATGGGAAGACACAAGCCGGAAAACATCGTCATCGAAATGGCAAGAGAAAACCAGACAACACAGAAGGGACAGAA
GAACAGCAGAGAAAGAATGAAGAGAATCGAAGAAGGAATCAAGGAACTGGGAAGCCAGATCCTGAAGGAACACCC
GGTCGAAAACACACAGCTGCAGAACGAAAAGCTGTACCTGTACTACCTGCAGAACGGAAGAGACATGTACGTCGA
CCAGGAACTGGACATCAACAGACTGAGCGACTACGACGTCGACCACATCGTCCCGCAGAGCTTCCTGAAGGACGA
CAGCATCGACAACAAGGTCCTGACAAGAAGCGACAAGAACAGAGGAAAGAGCGACAACGTCCCGAGCGAAGAAGT
CGTCAAGAAGATGAAGAACTACTGGAGACAGCTGCTGAACGCAAAGCTGATCACACAGAGAAAGTTCGACAACCT
GACAAAGGCAGAGAGAGGAGGACTGAGCGAACTGGACAAGGCAGGATTCATCAAGAGACAGCTGGTCGAAACAAG
ACAGATCACAAAGCACGTCGCACAGATCCTGGACAGCAGAATGAACACAAAGTACGACGAAAACGACAAGCTGAT
CAGAGAAGTCAAGGTCATCACACTGAAGAGCAAGCTGGTCAGCGACTTCAGAAAGGACTTCCAGTTCTACAAGGT
CAGAGAAATCAACAACTACCACCACGCACACGACGCATACCTGAACGCAGTCGTCGGAACAGCACTGATCAAGAA
GTACCCGAAGCTGGAAAGCGAATTCGTCTACGGAGACTACAAGGTCTACGACGTCAGAAAGATGATCGCAAAGAG
CGAACAGGAAATCGGAAAGGCAACAGCAAAGTACTTCTTCTACAGCAACATCATGAACTTCTTCAAGACAGAAAT
CACACTGGCAAACGGAGAAATCAGAAAGAGACCGCTGATCGAAACAAACGGAGAAACAGGAGAAATCGTCTGGGA
CAAGGGAAGAGACTTCGCAACAGTCAGAAAGGTCCTGAGCATGCCGCAGGTCAACATCGTCAAGAAGACAGAAGT
CCAGACAGGAGGATTCAGCAAGGAAAGCATCCTGCCGAAGAGAAACAGCGACAAGCTGATCGCAAGAAAGAAGGA
CTGGGACCCGAAGAAGTACGGAGGATTCGACAGCCCGACAGTCGCATACAGCGTCCTGGTCGTCGCAAAGGTCGA
AAAGGGAAAGAGCAAGAAGCTGAAGAGCGTCAAGGAACTGCTGGGAATCACAATCATGGAAAGAAGCAGCTTCGA
AAAGAACCCGATCGACTTCCTGGAAGCAAAGGGATACAAGGAAGTCAAGAAGGACCTGATCATCAAGCTGCCGAA
GTACAGCCTGTTCGAACTGGAAAACGGAAGAAAGAGAATGCTGGCAAGCGCAGGAGAACTGCAGAAGGGAAACGA
ACTGGCACTGCCGAGCAAGTACGTCAACTTCCTGTACCTGGCAAGCCACTACGAAAAGCTGAAGGGAAGCCCGGA
AGACAACGAACAGAAGCAGCTGTTCGTCGAACAGCACAAGCACTACCTGGACGAAATCATCGAACAGATCAGCGA
ATTCAGCAAGAGAGTCATCCTGGCAGACGCAAACCTGGACAAGGTCCTGAGCGCATACAACAAGCACAGAGACAA
GCCGATCAGAGAACAGGCAGAAAACATCATCCACCTGTTCACACTGACAAACCTGGGAGCACCGGCAGCATTCAA
GTACTTCGACACAACAATCGACAGAAAGAGATACACAAGCACAAAGGAAGTCCTGGACGCAACACTGATCCACCA
GAGCATCACAGGACTGTACGAAACAAGAATCGACCTGAGCCAGCTGGGAGGAGACGGAGGAGGAAGCCCGAAGAA
GAAGAGAAAGGTCTAGCTAGCCATCACATTTAAAAGCATCTCAGCCTACCATGAGAATAAGAGAAAGAAAATGAA
GATCAATAGCTTATTCATCTCTTTTTCTTTTTCGTTGGTGTAAAGCCAACACCCTGTCTAAAAAACATAAATTTC
TTTAATCATTTTGCCTCTTTTCTCTGTGCTTCAATTAATAAAAAATGGAAAGAACCTCGAG

### Lipid Nanoparticle (LNP) Formulation

In general, the lipid nanoparticle components were dissolved in 100% ethanol at various molar ratios. The RNA cargos (e.g., Cas9 mRNA and sgRNA) were dissolved in 25 mM citrate, 100 mM NaCl, pH 5.0, resulting in a concentration of RNA cargo of approximately 0.45 mg/mL. The LNPs used in Examples 2-4 contained ionizable lipid ((9Z,12Z)-3-((4,4-bis(octyloxy)butanoyl)oxy)-2-((((3-(diethylamino)propoxy)carbonyl)oxy)methyl)propyl octadeca-9,12-dienoate, also called 3-((4,4-bis(octyloxy)butanoyl)oxy)-2-((((3-(diethylamino)propoxy)carbonyl)oxy)methyl)propyl (9Z,12Z)-octadeca-9,12-dienoate), cholesterol, DSPC, and PEG2k-DMG in a 50:38:9:3 molar ratio, respectively. The LNPs were formulated with a lipid amine to RNA phosphate (N:P) molar ratio of about 6, and a ratio of gRNA to mRNA of 1:1 by weight. The LNPs used in Examples 2-4 contained Cas9 mRNA derived from SEQ ID NO: 501.

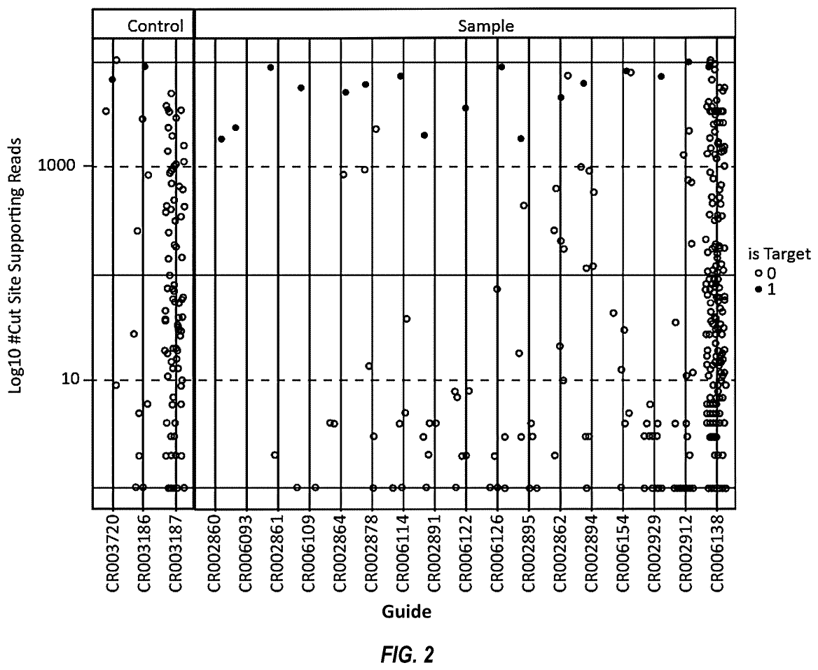

The LNPs were prepared using a cross-flow technique utilizing impinging jet mixing of the lipid in ethanol with two volumes of RNA solutions and one volume of water. The lipid in ethanol was mixed through a mixing cross with the two volumes of RNA solution. A fourth stream of water was mixed with the outlet stream of the cross through an inline tee (See WO2016010840 FIG. 2). The LNPs were held for 1 hour at room temperature, and further diluted with water (approximately 1:1 v/v). Diluted LNPs were concentrated using tangential flow filtration on a flat sheet cartridge (Sartorius, 100 kD MWCO) and then buffer exchanged using PD-10 desalting columns (GE) into 50 mM Tris, 45 mM NaCl, 5% (w/v) sucrose, pH 7.5 (TSS). The resulting mixture was then filtered using a 0.2 μm sterile filter. The final LNP was stored at 4° C. or −80° C. until further use.

Human HAO1 Guide Design and Human HAO1 with Cynomolgus Homology Guide Design

Initial guide selection was performed in silico using a human reference genome (e.g., hg38) and user defined genomic regions of interest (e.g., HAO1 protein coding exons), for identifying PAMs in the regions of interest. For each identified PAM, analyses were performed and statistics reported. gRNA molecules were further selected and rank-ordered based on a number of criteria known in the art (e.g., GC content, predicted on-target activity, and potential off-target activity).

A total of 146 guide RNAs were designed toward HAO1 (ENSG00000101323) targeting the protein coding regions within Exons 1, 2, 3, 4, 5, 6, 7, and 8. Guides and corresponding genomic coordinates are provided above (Table 1). Seventy-two of the guide RNAs have 100% homology with cynomolgus HAO1.

Cas9 (mRNA/Protein) and Guide RNA Delivery In Vitro

The human embryonic kidney adenocarcinoma cell line HEK293 constitutively expressing Spy Cas9 (“HEK293_Cas9”) was cultured in DMEM media supplemented with 10% fetal bovine serum and 500 μg/ml G418. Cells were plated at a density of 10,000 cells/well in a 96-well plate 20 hours prior to transfection (˜70% confluent at time of transfection). Cells were transfected with Lipofectamine RNAiMAX (ThermoFisher, Cat. 13778150) according to the manufacturer's protocol. Cells were transfected with a lipoplex containing individual guide (25 nM), trRNA (25 nM), Lipofectamine RNAiMAX (0.3 μL/well) and OptiMem.

The human hepatocellular carcinoma cell line HUH7 (Japanese Collection of Research Bioresources Cell Bank, Cat. JCRB0403) was cultured in DMEM media supplemented with 10% fetal bovine serum. Cells were plated at a density of 15,000 cells/well in a 96-well plate 20 hours prior to transfection (˜70% confluent at time of transfection). Cells were transfected with Lipofectamine MessengerMAX (ThermoFisher, Cat. LMRNA003) according to the manufacturer's protocol. Cells were sequentially transfected with a lipoplex containing Spy Cas9 mRNA (100 ng; SEQ ID No:500), MessengerMAX (0.3 μL/well) and OptiMem followed by a separate lipoplex containing individual guide (25 nM), tracer RNA (25 nM), MessengerMAX (0.3 μL/well) and OptiMem.

Primary human liver hepatocytes (PHH) (Gibco, Lot #s Hu8249 and Hu8298) and primary cynomolgus liver hepatocytes (PCH) (Gibco, Lot #Cy367) were thawed and resuspended in hepatocyte thawing medium with supplements (Gibco, Cat. CM7500) followed by centrifugation. The supernatant was discarded and the pelleted cells resuspended in hepatocyte plating medium plus supplement pack (Invitrogen, Cat. A1217601 and CM3000). Cells were counted and plated on Bio-coat collagen I coated 96-well plates (ThermoFisher, Cat. 877272) at a density of 33,000 cells/well for PHH and 50,000 cells/well for PCH. Plated cells were allowed to settle and adhere for 5 hours in a tissue culture incubator at 37° C. and 5% CO₂ atmosphere. After incubation cells were checked for monolayer formation and were washed once with hepatocyte culture medium (Takara, Cat. Y20020 and/or Invitrogen, Cat. A1217601 and CM4000).

For studies utilizing dgRNAs, individual crRNA and trRNA was pre-annealed by mixing equivalent amounts of reagent and incubating at 95° C. for 2 min and cooling to room temperature. The dual guide (dgRNA) consisting of pre-annealed crRNA and trRNA, was incubated with Spy Cas9 protein to form a ribonucleoprotein (RNP) complex. Cells were transfected with Lipofectamine RNAiMAX (ThermoFisher, Cat. 13778150) according to the manufacturer's protocol. Cells were transfected with an RNP containing Spy Cas9 (10 nM), individual guide (10 nM), tracer RNA (10 nM), Lipofectamine RNAiMAX (1.0 μL/well) and OptiMem.

Primary human and cyno hepatocytes were also treated with LNPs as further described below. Cells were incubated at 37° C., 5% CO₂ for 48 hours prior to treatment with LNPs. LNPs were incubated in media containing 6% cynomolgus serum at 37° C. for 10 minutes and administered to cells in amounts as further provided herein.

### Genomic DNA Isolation

HEK293_Cas9, HUH7, PHH, and PCH transfected cells were harvested post-transfection at 24, 48, 72, or 96 hours. The gDNA was extracted from each well of a 96-well plate using 50 μL/well BuccalAmp DNA Extraction solution (Epicentre, Cat. QE09050) according to manufacturer's protocol. All DNA samples were subjected to PCR and subsequent NGS analysis, as described herein.

### Next-Generation Sequencing (“NGS”) and Analysis for On-Target Cleavage Efficiency

To quantitatively determine the efficiency of editing at the target location in the genome, deep sequencing was utilized to identify the presence of insertions and deletions introduced by gene editing. PCR primers were designed around the target site within the gene of interest (e.g. HAO1), and the genomic area of interest was amplified. Primer sequence design was done as is standard in the field.

Additional PCR was performed according to the manufacturer's protocols (Illumina) to add chemistry for sequencing. The amplicons were sequenced on an Illumina MiSeq instrument. The reads were aligned to the human reference genome (e.g., hg38) after eliminating those having low quality scores. The resulting files containing the reads were mapped to the reference genome (BAM files), where reads that overlapped the target region of interest were selected and the number of wild type reads versus the number of reads which contain an insertion or deletion (“indel”) was calculated.

The editing percentage (e.g., the “editing efficiency” or “percent editing”) is defined as the total number of sequence reads with insertions or deletions (“indels”) over the total number of sequence reads, including wild type.

### HAO1 Transcript Analysis by Quantitative PCR

As further described in Example 3, primary human hepatocytes and primary cynomolgus hepatocytes were treated with LNPs formulated with select modified guides from Table 2. LNPs were incubated in media (Takara, Cat. Y20020) containing 3% cynomolgus serum at 37° C. for 10 minutes. Post-incubation the LNPs were added to the human or cynomolgus hepatocytes. Twenty-one days post-treatment, the media was removed and 50 μL/well TripleE (Gibco, Cat 12563-029) was added to the cells which were then incubated 37° C. for 10 minutes. 50 μL/well of media was added to the cells to quench the TripleE and dissociate the cells from the plate. The cells were then centrifuged at 2,000 rpm to pellet and the supernatant was aspirated from the samples. To isolate mRNA, the Qiagen RNeasy Mini Kit (Qiagen, Cat. 74106) was used. The RNeasy Mini Kit procedure was completed according to the manufacturer's protocol. RNA was quantified using a Nanodrop 8000 (Thermofisher Scientific, Cat. ND-8000-GL). The RNA quantification procedure was completed according to the manufacturer's protocol. RNA samples were stored at −20° C. prior to use.

Quantitative PCR was performed to assess HAO1 transcript levels. The Taqman RNA-to-Ct 1-Step Kit (Thermo Fisher Scientific, Cat. 4392938) was used to create the PCR reactions. The reaction set-up was completed according to the manufacturer's protocol. Quantitative PCR probes targeting HAO1 (Thermo Fisher Scientific, Cat. 4351372, transcript UniGene ID Hs01023324_g1) and 18S (Thermo Fisher Scientific, Cat. 4319413E) were used in the PCR reactions. The StepOnePlus Real-Time PCR System (Thermo Fisher Scientific, Cat. 4376600) was used to perform the real-time PCR reaction and transcript quantification according to the manufacturer's protocol.

### Glycolate Oxidase (GO) Protein Analysis by Western Blot

Primary human hepatocytes and primary cynomolgus hepatocytes were treated with LNP formulated with select guides from Table 2 as further described in Example 3. LNPs were incubated in media (Takara, Cat. Y20020) containing 3% cynomolgus serum at 37° C. for 10 minutes. Post-incubation the LNPs were added to the human or cynomolgus hepatocytes. Twenty-one days post-transfection, the media was removed and the cells were lysed with 50 μL/well RIPA buffer (Boston Bio Products, Cat. BP-115) plus freshly added protease inhibitor mixture consisting of complete protease inhibitor cocktail (Sigma, Cat. 11697498001), 1 mM DTT, and 250 U/ml Benzonase (EMD Millipore, Cat. 71206-3). Cells were kept on ice for 30 minutes at which time NaCl (1 M final concentration) was added. Cell lysates were thoroughly mixed and retained on ice for 30 minutes. The whole cell extracts (“WCE”) were transferred to a PCR plate and centrifuged to pellet debris. A Bradford assay (Bio-Rad, Cat. 500-0001) was used to assess protein content of the lysates. The Bradford assay procedure was completed according to the manufacturer's protocol. Extracts were stored at −20° C. prior to use.

AGT-deficient mice were treated with LNP formulated with select guides as further described in Example 4. Livers were harvested from the mice post-treatment and 60 mg portions were used for protein extraction. The samples were placed in bead tubes (MP Biomedical, Cat. 6925-500) and lysed with 600 μL/sample of RIPA buffer (Boston Bio Products, Cat. BP-115) plus freshly added protease inhibitor mixture consisting of complete protease inhibitor cocktail (Sigma, Cat. 116974500) and homogenized at 5.0 m/sec. The samples were then centrifuged at 14,000 RPM for 10 min. at 4° C. and the liquid was transferred to a new tube. A final centrifugation was performed at 14,000 RPM for 10 min. and the samples were quantified using a Bradford assay as described above.

Western blots were performed to assess GO protein levels. Lysates were mixed with Laemmli buffer and denatured at 95° C. for 10 minutes. Western blots were run using the NuPage system on 4-12% Bis-Tris gels (Thermo Fisher Scientific, Cat. NP0323BOX) according to the manufacturer's protocol followed by wet transfer onto 0.45 μm nitrocellulose membrane (Bio-Rad, Cat. 1620115). After transfer membranes were rinsed thoroughly with water and stained with Ponceau S solution (Boston Bio Products, Cat. ST-180) to confirm complete and even transfer. Blots were blocked using 5% Dry Milk in TBS for 30 minutes on a lab rocker at room temperature. Blots were rinsed with TBST and probed with rabbit α-GO polyclonal antibody (Genetex, Cat. GTX81144) at 1:1000 in TBST. For blots with in vitro cell lysate, vinculin was used as a loading control (Abcam, ab130007) at 1:1000 in TBST and incubated simultaneously with the GO primary antibody. For blots with in vivo mouse liver extracts, alpha-tubulin was used as a loading control (Abcam, ab7291) at 1:1000 in TBST and incubated simultaneously with the GO primary antibody. Blots were sealed in a bag and kept overnight at 4° C. on a lab rocker. After incubation, blots were rinsed 3 times for 5 minutes each in TBST and probed with secondary antibodies to Mouse and Rabbit (Thermo Fisher Scientific, Cat. PI35518 and PISA535571) at 1:12,500 each in TBST for 30 minutes at room temperature. After incubation, blots were rinsed 3 times for 5 minutes each in TBST and 2 times with PBS. Blots were visualized and analyzed using a Licor Odyssey system.

### Example 2—Screening and Guide Qualification

### Cross Screening of HAO1 Guides in Multiple Cell Types

Guides targeting human HAO1 and those with homology in cynomolgus monkey were transfected into the HEK293_Cas9 and HUH7 cell lines, as well as primary human and cynomolgus hepatocytes as described in Example 1. Percent editing was determined for crRNAs comprising each guide sequence across each cell type. The screening data for the guide sequences in Table 1 in all four cell lines are listed below (Tables 7B-10).

Table 7B shows the average and standard deviation of triplicate samples for % Edit, % Insertion (Ins), and % Deletion (Del) for the HAO1 and control dgRNAs (Table 7A) in the human kidney adenocarcinoma cell line, HEK293_Cas9, which constitutively over expresses Spy Cas9 protein.

TABLE 7A
Control non-HAO1 guides

| | | SEQ |
|---|---|---|
| | | ID |
| Guide ID | SEQUENCE | NO: |
| CR001261 | GCCAGACUCCAAGUUCUGCC | 147 |
| CR001262 | UAAGGCCAGUGGAAAGAAUU | 148 |
| CR001263 | GGCAGCGAGGAGUCCACAGU | 149 |
| CR001264 | UCUUUCCACUGGCCUUAACC | 150 |

TABLE 7B
HAO1 editing data for crRNAs delivered to HEK293_Cas9 cells

| | Avg | Std Dev | Avg | Std Dev | Avg | Std Dev |
|---|---|---|---|---|---|---|
| Guide ID | % Edit | % Edit | % Ins | % Ins | % Del | % Del |
| CR001261 | 57.63 | 7.78 | 44.39 | 5.77 | 13.24 | 2.01 |
| CR001262 | 45.05 | 4.72 | 5.06 | 0.77 | 39.99 | 4.11 |
| CR001263 | 20.34 | 6.88 | 1.77 | 0.85 | 18.56 | 6.03 |
| CR001264 | 51.21 | 14.70 | 10.33 | 2.35 | 40.88 | 12.52 |
| CR002857 | 10.81 | 1.99 | 1.98 | 0.78 | 8.83 | 1.21 |
| CR002858 | 14.04 | 6.71 | 2.58 | 1.42 | 11.45 | 5.33 |
| CR002859 | 10.67 | 4.25 | 2.71 | 0.97 | 7.96 | 3.27 |
| CR002860 | 32.75 | 8.30 | 7.28 | 2.18 | 25.47 | 6.13 |
| CR002861 | 42.99 | 6.61 | 7.29 | 1.37 | 35.70 | 5.28 |
| CR002862 | 33.06 | 8.57 | 7.44 | 0.56 | 25.62 | 8.02 |
| CR002863 | 41.26 | 18.14 | 3.94 | 1.72 | 37.33 | 16.42 |
| CR002864 | 41.13 | 5.50 | 2.10 | 0.34 | 39.03 | 5.17 |
| CR002865 | 39.36 | 11.78 | 26.95 | 7.67 | 12.41 | 4.17 |
| CR002866 | 23.78 | 6.61 | 5.81 | 1.53 | 17.97 | 5.16 |
| CR002867 | 17.93 | 1.80 | 9.03 | 1.40 | 8.90 | 0.41 |
| CR002868 | 4.31 | 1.19 | 0.16 | 0.06 | 4.16 | 1.19 |
| CR002869 | 11.20 | 5.33 | 1.52 | 0.62 | 9.68 | 4.71 |
| CR002870 | 13.00 | 4.44 | 4.62 | 1.92 | 8.38 | 2.58 |
| CR002871 | 6.05 | 1.29 | 0.75 | 0.47 | 5.30 | 1.01 |
| CR002872 | 5.01 | 1.21 | 0.26 | 0.06 | 4.75 | 1.25 |
| CR002873 | 17.86 | 3.15 | 3.45 | 1.13 | 14.40 | 2.27 |
| CR002874 | 11.10 | 2.35 | 2.14 | 0.66 | 8.97 | 1.70 |
| CR002875 | 2.35 | 0.29 | 0.16 | 0.05 | 2.18 | 0.24 |
| CR002876 | 24.01 | 8.59 | 2.67 | 0.98 | 21.34 | 7.61 |
| CR002877 | 34.59 | 9.11 | 4.30 | 1.35 | 30.29 | 7.75 |
| CR002878 | 44.53 | 9.84 | 32.55 | 6.27 | 11.98 | 3.73 |
| CR002879 | 23.90 | 9.03 | 3.58 | 1.40 | 20.32 | 7.63 |
| CR002880 | 25.94 | 10.25 | 5.09 | 2.32 | 20.84 | 7.93 |
| CR002881 | 14.07 | 3.76 | 3.08 | 0.98 | 10.98 | 2.79 |
| CR002882 | 9.49 | 2.54 | 0.98 | 0.34 | 8.51 | 2.27 |
| CR002883 | 24.68 | 8.44 | 2.47 | 0.84 | 22.22 | 7.61 |
| CR002884 | 24.90 | 4.72 | 4.84 | 1.07 | 20.06 | 4.06 |
| CR002885 | 4.48 | 1.31 | 0.71 | 0.45 | 3.77 | 0.86 |
| CR002886 | 21.81 | 4.79 | 1.42 | 0.21 | 20.39 | 4.60 |
| CR002887 | 30.22 | 8.87 | 4.75 | 1.78 | 25.47 | 7.16 |
| CR002888 | 16.67 | 3.86 | 3.01 | 0.82 | 13.66 | 3.09 |
| CR002889 | 27.12 | 4.44 | 7.22 | 1.14 | 19.89 | 3.43 |
| CR002890 | 4.99 | 1.62 | 1.24 | 0.36 | 3.75 | 1.27 |
| CR002892 | 1.78 | 0.08 | 0.24 | 0.06 | 1.55 | 0.04 |
| CR002893 | 2.52 | 0.27 | 0.24 | 0.06 | 2.27 | 0.26 |
| CR002894 | 48.00 | 9.08 | 5.08 | 1.43 | 42.92 | 9.25 |
| CR002895 | 45.92 | 9.21 | 27.11 | 5.31 | 18.82 | 3.92 |
| CR002896 | 36.73 | 11.14 | 14.03 | 4.17 | 22.71 | 7.54 |
| CR002897 | 15.08 | 4.28 | 10.19 | 3.31 | 4.89 | 0.97 |
| CR002898 | 9.39 | 1.61 | 0.75 | 0.21 | 8.64 | 1.40 |
| CR002899 | 14.00 | 4.15 | 6.18 | 2.37 | 7.82 | 1.79 |
| CR002900 | 32.51 | 5.89 | 6.27 | 0.76 | 26.24 | 5.15 |
| CR002901 | 11.64 | 5.30 | 3.25 | 1.82 | 8.39 | 3.53 |
| CR002902 | 5.28 | 1.89 | 1.37 | 0.67 | 3.91 | 1.23 |
| CR002903 | 5.43 | 1.24 | 2.57 | 0.59 | 2.86 | 0.65 |
| CR002904 | 22.22 | 6.33 | 4.65 | 1.26 | 17.57 | 5.09 |
| CR002905 | 18.99 | 6.09 | 5.64 | 1.94 | 13.35 | 4.17 |
| CR002906 | 21.81 | 7.92 | 7.55 | 2.36 | 14.26 | 5.58 |
| CR002907 | 10.93 | 4.07 | 1.74 | 0.57 | 9.20 | 3.52 |
| CR002908 | 12.03 | 4.13 | 3.15 | 1.11 | 8.88 | 3.03 |
| CR002909 | 6.46 | 1.01 | 2.35 | 0.55 | 4.11 | 0.47 |
| CR002910 | 19.20 | 7.31 | 6.37 | 2.18 | 12.83 | 5.16 |
| CR002911 | 22.08 | 4.51 | 7.84 | 1.25 | 14.24 | 3.26 |
| CR002912 | 31.20 | 10.73 | 1.80 | 0.25 | 29.39 | 10.48 |
| CR002913 | 7.47 | 2.79 | 4.37 | 1.88 | 3.10 | 0.92 |
| CR002914 | 3.35 | 1.26 | 0.21 | 0.13 | 3.14 | 1.13 |
| CR002915 | 25.49 | 10.72 | 5.31 | 2.22 | 20.17 | 8.50 |
| CR002916 | 4.02 | 0.76 | 0.48 | 0.11 | 3.54 | 0.64 |
| CR002917 | 5.66 | 1.08 | 0.64 | 0.41 | 5.02 | 1.17 |
| CR002918 | 1.73 | 0.05 | 0.07 | 0.02 | 1.66 | 0.04 |
| CR002919 | 8.90 | 1.36 | 1.28 | 0.40 | 7.62 | 0.98 |
| CR002920 | 10.71 | 3.10 | 1.34 | 0.33 | 9.37 | 2.86 |
| CR002921 | 17.36 | 5.85 | 1.60 | 0.47 | 15.76 | 5.38 |
| CR002922 | 28.05 | 4.17 | 7.66 | 1.25 | 20.40 | 2.94 |
| CR002923 | 13.61 | 3.45 | 6.18 | 1.63 | 7.43 | 1.83 |
| CR002924 | 9.00 | 3.04 | 2.42 | 0.79 | 6.58 | 2.30 |
| CR002925 | 4.85 | 1.26 | 0.73 | 0.15 | 4.12 | 1.21 |
| CR002926 | 8.97 | 1.73 | 0.93 | 0.35 | 8.04 | 1.42 |
| CR002927 | 23.16 | 7.11 | 12.70 | 3.73 | 10.46 | 3.39 |
| CR002928 | 10.44 | 3.86 | 2.20 | 0.73 | 8.24 | 3.14 |
| CR002929 | 28.18 | 7.04 | 1.66 | 0.59 | 26.52 | 6.84 |
| CR002930 | 21.56 | 7.43 | 10.51 | 3.62 | 11.05 | 3.81 |
| CR002931 | 28.08 | 5.00 | 2.87 | 1.90 | 25.20 | 3.10 |
| CR002932 | 22.21 | 5.93 | 6.22 | 2.01 | 15.98 | 4.11 |
| CR002933 | 34.74 | 6.55 | 16.32 | 2.48 | 18.43 | 4.09 |
| CR002934 | 9.78 | 2.17 | 1.63 | 0.24 | 8.15 | 1.97 |
| CR002935 | 16.22 | 3.29 | 3.39 | 0.75 | 12.84 | 2.57 |
| CR002936 | 15.18 | 2.98 | 6.73 | 1.39 | 8.46 | 1.61 |
| CR002937 | 17.39 | 0.89 | 3.88 | 0.45 | 13.50 | 0.47 |
| CR002938 | 23.30 | 5.53 | 4.92 | 0.97 | 18.37 | 4.57 |

Table 8 shows the % Edit, % Insertion (Ins), and % Deletion (Del) for the tested HAO1 and control dgRNAs (Table 7A) co-transfected with Spy Cas9 mRNA in the human hepatocellular carcinoma cell line, HUH7. N=1.

TABLE 8
HAO1 editing data for crRNAs delivered to HUH7 cells

| Guide ID | % Edit | % Ins | % Del |
|---|---|---|---|
| CR001261 | 91.58 | 67.48 | 24.11 |
| CR001262 | 66.17 | 5.97 | 60.19 |
| CR001263 | 65.92 | 3.25 | 62.67 |
| CR001264 | 86.57 | 15.37 | 71.19 |
| CR002857 | 7.89 | 1.42 | 6.47 |
| CR002858 | 40.74 | 5.60 | 35.14 |
| CR002859 | 37.04 | 9.02 | 28.02 |
| CR002860 | 32.18 | 7.09 | 25.09 |
| CR002861 | 32.23 | 5.11 | 27.12 |
| CR002862 | 28.20 | 5.19 | 23.01 |
| CR002863 | 56.91 | 5.59 | 51.31 |
| CR002864 | 27.47 | 1.18 | 26.29 |
| CR002865 | 39.30 | 26.79 | 12.51 |
| CR002866 | 29.67 | 8.06 | 21.61 |
| CR002867 | 26.79 | 11.42 | 15.36 |
| CR002868 | 11.58 | 0.40 | 11.18 |
| CR002869 | 18.31 | 2.68 | 15.64 |
| CR002870 | 17.16 | 5.69 | 11.47 |
| CR002871 | 19.28 | 1.29 | 17.99 |
| CR002872 | 12.78 | 0.32 | 12.46 |
| CR002873 | 48.63 | 7.70 | 40.93 |
| CR002874 | 22.37 | 2.34 | 20.03 |
| CR002875 | 5.83 | 0.34 | 5.50 |
| CR002876 | 32.63 | 4.29 | 28.34 |
| CR002877 | 46.74 | 4.46 | 42.28 |
| CR002878 | 49.04 | 37.05 | 12.00 |
| CR002879 | 42.67 | 5.64 | 37.02 |
| CR002880 | 57.41 | 11.34 | 46.07 |
| CR002881 | 36.09 | 8.29 | 27.80 |
| CR002882 | 31.37 | 2.18 | 29.18 |
| CR002883 | 59.63 | 8.08 | 51.55 |
| CR002884 | 56.45 | 10.28 | 46.16 |
| CR002885 | 7.34 | 1.13 | 6.20 |
| CR002886 | 45.72 | 2.68 | 43.04 |
| CR002887 | 64.77 | 8.09 | 56.68 |
| CR002888 | 49.58 | 5.66 | 43.92 |
| CR002889 | 27.43 | 8.68 | 18.74 |
| CR002890 | 20.31 | 5.84 | 14.47 |
| CR002891 | 48.18 | 2.40 | 45.78 |
| CR002892 | 2.40 | 0.58 | 1.82 |
| CR002893 | 4.87 | 0.38 | 4.49 |
| CR002894 | 41.82 | 3.45 | 38.38 |
| CR002895 | 37.36 | 17.03 | 20.34 |
| CR002896 | 62.99 | 21.92 | 41.07 |
| CR002897 | 25.96 | 14.78 | 11.18 |
| CR002898 | 13.82 | 1.32 | 12.51 |
| CR002899 | 33.32 | 10.36 | 22.97 |
| CR002900 | 38.69 | 9.25 | 29.44 |
| CR002901 | 30.65 | 7.61 | 23.03 |
| CR002902 | 31.83 | 7.16 | 24.67 |
| CR002903 | 31.85 | 8.09 | 23.76 |
| CR002904 | 66.95 | 18.97 | 47.98 |
| CR002905 | 37.51 | 12.24 | 25.28 |
| CR002906 | 43.69 | 13.14 | 30.55 |
| CR002907 | 14.14 | 2.15 | 11.99 |
| CR002908 | 32.07 | 5.31 | 26.76 |
| CR002909 | 24.19 | 7.83 | 16.37 |
| CR002910 | 44.37 | 12.87 | 31.49 |
| CR002911 | 32.24 | 9.40 | 22.84 |
| CR002912 | 60.89 | 2.43 | 58.45 |
| CR002913 | 32.21 | 20.12 | 12.09 |
| CR002914 | 19.13 | 0.61 | 18.51 |
| CR002915 | 45.51 | 5.22 | 40.29 |
| CR002916 | 9.61 | 0.63 | 8.97 |
| CR002917 | 12.41 | 1.44 | 10.97 |
| CR002918 | 2.03 | 0.08 | 1.95 |
| CR002919 | 24.33 | 3.63 | 20.71 |
| CR002920 | 16.86 | 2.86 | 14.00 |
| CR002921 | 40.74 | 2.99 | 37.75 |
| CR002922 | 47.63 | 14.63 | 33.00 |
| CR002923 | 13.50 | 6.52 | 6.98 |
| CR002924 | 43.59 | 9.85 | 33.74 |
| CR002925 | 42.21 | 5.16 | 37.05 |
| CR002926 | 25.31 | 2.67 | 22.65 |
| CR002927 | 62.52 | 27.90 | 34.62 |
| CR002928 | 32.58 | 5.67 | 26.91 |
| CR002929 | 75.94 | 1.83 | 74.11 |
| CR002930 | 52.99 | 19.69 | 33.30 |
| CR002931 | 47.32 | 2.87 | 44.45 |
| CR002932 | 56.46 | 9.72 | 46.73 |
| CR002933 | 47.74 | 22.21 | 25.53 |
| CR002934 | 50.50 | 8.15 | 42.35 |
| CR002935 | 43.84 | 6.48 | 37.36 |
| CR002936 | 40.36 | 16.67 | 23.69 |
| CR002937 | 44.96 | 6.27 | 38.68 |
| CR002938 | 43.43 | 6.30 | 37.12 |
| CR006092 | 65.7 | 22.4 | 43.8 |
| CR006093 | 70 | 4.4 | 66.1 |
| CR006094 | 31.5 | 3 | 28.8 |
| CR006095 | 32.3 | 12.8 | 20 |
| CR006096 | 0.5 | 0 | 0.5 |
| CR006097 | 0.2 | 0.1 | 0.2 |
| CR006098 | 19.8 | 4.4 | 15.6 |
| CR006099 | 32.8 | 9.2 | 24.3 |
| CR006100 | 18.3 | 9.6 | 9.1 |
| CR006101 | 43.7 | 3.3 | 40.9 |
| CR006102 | 33.7 | 17.9 | 16.2 |
| CR006103 | 63.1 | 5.5 | 58.4 |
| CR006104 | 23.4 | 2.2 | 21.6 |
| CR006105 | 39 | 22.2 | 17.4 |
| CR006106 | 39.9 | 22.1 | 18.4 |
| CR006107 | 48 | 24.4 | 24.3 |
| CR006108 | 43.3 | 2.8 | 41.2 |
| CR006109 | 51.8 | 4.6 | 47.5 |
| CR006110 | 11.3 | 5.5 | 5.9 |
| CR006111 | 4.4 | 0.8 | 3.7 |
| CR006112 | 32.1 | 3.1 | 29.5 |
| CR006113 | 30 | 4.8 | 25.9 |
| CR006114 | 63 | 24.7 | 39.2 |
| CR006115 | 61.3 | 15.1 | 46.6 |
| CR006116 | 56.6 | 19 | 38.4 |
| CR006117 | 22.8 | 7.1 | 16 |
| CR006118 | 48.3 | 20.2 | 28.6 |
| CR006119 | 21.8 | 3.3 | 18.6 |
| CR006120 | 31.1 | 13.7 | 17.8 |
| CR006121 | 36.5 | 16.9 | 20.3 |
| CR006122 | 36.5 | 6.8 | 30 |
| CR006123 | 49.8 | 15.1 | 35.6 |
| CR006124 | 60.1 | 5.7 | 55.3 |
| CR006125 | 58.1 | 22.5 | 36.8 |
| CR006126 | 69 | 6.8 | 62.8 |
| CR006127 | 46.7 | 7.9 | 39.8 |
| CR006128 | 22.4 | 2.1 | 20.5 |
| CR006129 | 44.6 | 13.2 | 32.3 |
| CR006133 | 29.6 | 17.4 | 12.6 |
| CR006134 | 37.3 | 2.4 | 35 |
| CR006135 | 55 | 17 | 38.9 |
| CR006136 | 52.6 | 39.4 | 13.4 |
| CR006137 | 45.5 | 4.8 | 41.8 |

Table 9 shows the average and standard deviation for % Edit, % Insertion (Ins), and % Deletion (Del) for the tested HAO1 and control dgRNAs (Table 7A) co-transfected with Spy Cas9 protein in primary human hepatocytes. N=3.

TABLE 9
HAO1 editing data for crRNAs delivered
to primary human hepatocytes

| GUIDE | Avg | Std Dev | Avg | Std Dev | Avg | Std Dev |
|---|---|---|---|---|---|---|
| ID | % Edit | % Edit | % Ins | % Ins | % Del | % Del |
| CR001261 | 24.97 | 11.07 | 17.63 | 8.65 | 21.97 | 9.63 |
| CR001262 | 24.70 | 18.60 | 29.03 | 21.29 | 22.67 | 16.57 |
| CR001263 | 4.60 | 3.24 | 10.30 | 8.75 | 4.53 | 3.75 |
| CR001264 | 25.70 | 11.14 | 30.90 | 12.51 | 24.03 | 10.19 |
| CR002858 | 7.47 | 5.38 | 5.60 | 2.81 | 5.20 | 2.19 |
| CR002859 | 10.73 | 8.36 | 6.63 | 4.65 | 7.70 | 5.57 |
| CR002860 | 33.40 | 26.12 | 38.50 | 28.05 | 41.77 | 30.94 |
| CR002861 | 23.77 | 17.31 | 22.60 | 15.04 | 21.97 | 14.34 |
| CR002862 | 18.50 | 14.57 | 21.20 | 16.41 | 19.67 | 16.17 |
| CR002863 | 8.70 | 6.52 | 7.77 | 5.96 | 12.80 | 9.48 |
| CR002864 | 22.47 | 18.77 | 16.23 | 13.97 | 23.00 | 19.14 |
| CR002865 | 4.03 | 1.82 | 3.80 | 1.80 | 3.37 | 1.46 |
| CR002866 | 4.90 | 3.65 | 2.87 | 1.83 | 2.03 | 1.59 |
| CR002867 | 7.47 | 3.97 | 5.40 | 3.24 | 4.60 | 2.31 |
| CR002869 | 1.00 | 0.87 | 1.00 | 0.50 | 0.70 | 0.30 |
| CR002870 | 0.87 | 0.51 | 1.10 | 0.17 | 0.27 | 0.23 |
| CR002873 | 4.43 | 2.37 | 4.47 | 3.27 | 3.00 | 1.85 |
| CR002874 | 3.53 | 2.97 | 2.87 | 2.48 | 4.00 | 3.46 |
| CR002875 | 0.07 | 0.06 | 0.37 | 0.12 | 0.77 | 0.58 |
| CR002876 | 0.10 | 0.00 | 0.53 | 0.46 | 0.80 | 0.69 |
| CR002877 | 3.43 | 2.80 | 3.40 | 2.44 | 3.80 | 3.03 |
| CR002878 | 19.57 | 13.17 | 11.80 | 6.71 | 15.40 | 9.28 |
| CR002879 | 12.13 | 8.28 | 6.90 | 4.50 | 7.53 | 5.00 |
| CR002880 | 3.33 | 1.47 | 5.73 | 2.70 | 4.60 | 2.46 |
| CR002881 | 2.20 | 1.21 | 0.67 | 0.40 | 1.47 | 0.93 |
| CR002882 | 5.73 | 4.97 | 4.60 | 3.81 | 3.47 | 2.75 |
| CR002883 | 3.67 | 2.67 | 3.37 | 2.74 | 2.27 | 1.71 |
| CR002884 | 5.60 | 4.85 | 8.23 | 6.52 | 3.80 | 2.94 |
| CR002885 | 0.07 | 0.06 | 0.97 | 0.23 | 0.70 | 0.26 |
| CR002886 | 2.87 | 1.69 | 3.07 | 2.40 | 5.33 | 4.36 |
| CR002887 | 11.27 | 9.33 | 8.67 | 6.65 | 9.93 | 8.00 |
| CR002888 | 3.07 | 2.66 | 4.07 | 2.22 | 2.50 | 2.08 |
| CR002889 | 1.67 | 1.27 | 1.83 | 0.91 | 1.20 | 0.78 |
| CR002890 | 4.63 | 3.41 | 3.07 | 1.69 | 4.07 | 3.27 |
| CR002892 | 1.00 | 0.61 | 0.37 | 0.15 | 0.40 | 0.10 |
| CR002893 | 1.00 | 0.87 | 1.40 | 1.21 | 0.43 | 0.29 |
| CR002894 | 33.80 | 25.05 | 37.17 | 29.11 | 42.20 | 31.31 |
| CR002895 | 15.27 | 6.35 | 20.43 | 8.78 | 17.43 | 7.34 |
| CR002896 | 6.70 | 4.88 | 2.03 | 1.10 | 4.17 | 2.10 |
| CR002897 | 0.60 | 0.26 | 2.10 | 0.89 | 1.37 | 0.55 |
| CR002898 | 2.40 | 2.08 | 2.83 | 2.37 | 2.90 | 2.25 |
| CR002899 | 0.63 | 0.25 | 0.60 | 0.26 | 0.33 | 0.21 |
| CR002900 | 6.67 | 5.77 | 6.83 | 4.67 | 12.60 | 8.84 |
| CR002901 | 10.33 | 8.95 | 4.87 | 3.63 | 3.33 | 2.47 |
| CR002902 | 5.63 | 4.29 | 10.00 | 8.40 | 3.80 | 3.29 |
| CR002903 | 1.93 | 1.59 | 4.07 | 3.27 | 3.97 | 3.18 |
| CR002904 | 9.70 | 7.22 | 7.30 | 4.73 | 10.97 | 7.95 |
| CR002905 | 2.97 | 2.23 | 3.97 | 2.75 | 2.73 | 1.19 |
| CR002906 | 1.93 | 1.06 | 3.07 | 1.37 | 1.77 | 0.51 |
| CR002907 | 0.90 | 0.69 | 3.33 | 1.00 | 3.00 | 1.22 |
| CR002908 | 1.63 | 1.16 | 1.00 | 0.87 | 1.20 | 0.56 |
| CR002909 | 4.77 | 3.53 | 2.60 | 1.65 | 2.23 | 1.25 |
| CR002910 | 4.13 | 2.84 | 4.60 | 3.06 | 4.13 | 2.47 |
| CR002911 | 4.93 | 3.60 | 4.20 | 3.30 | 3.83 | 2.89 |
| CR002912 | 24.73 | 21.33 | 24.03 | 20.73 | 26.83 | 23.15 |
| CR002913 | 1.47 | 0.70 | 0.60 | 0.26 | 0.80 | 0.46 |
| CR002914 | 0.87 | 0.59 | 0.70 | 0.35 | 0.33 | 0.29 |
| CR002915 | 5.50 | 3.84 | 9.53 | 6.79 | 6.37 | 3.95 |
| CR002916 | 1.63 | 1.17 | 2.00 | 0.92 | 1.87 | 1.01 |
| CR002917 | 2.27 | 1.33 | 2.13 | 1.85 | 0.77 | 0.49 |
| CR002918 | 0.27 | 0.15 | 0.13 | 0.12 | 0.20 | 0.17 |
| CR002920 | 0.40 | 0.35 | 0.80 | 0.69 | 0.27 | 0.23 |
| CR002921 | 2.03 | 1.67 | 1.13 | 0.98 | 2.43 | 1.85 |
| CR002922 | 6.37 | 4.44 | 6.43 | 4.80 | 4.73 | 3.09 |
| CR002923 | 0.97 | 0.59 | 1.47 | 0.87 | 0.20 | 0.17 |
| CR002924 | 1.80 | 1.08 | 3.33 | 2.19 | 2.07 | 0.83 |
| CR002925 | 2.07 | 1.62 | 0.33 | 0.29 | 1.00 | 0.87 |
| CR002926 | 0.33 | 0.29 | 1.63 | 1.33 | 0.90 | 0.69 |
| CR002927 | 7.40 | 5.32 | 11.33 | 6.66 | 6.73 | 5.40 |
| CR002928 | 4.07 | 2.78 | 4.60 | 2.62 | 3.70 | 2.54 |
| CR002929 | 23.60 | 20.18 | 32.87 | 27.95 | 24.83 | 21.25 |
| CR002930 | 2.17 | 0.90 | 3.57 | 2.33 | 4.80 | 3.49 |
| CR002931 | 3.67 | 3.00 | 4.40 | 3.47 | 3.73 | 2.73 |
| CR002932 | 4.47 | 2.75 | 4.47 | 2.97 | 5.20 | 3.52 |
| CR002933 | 4.67 | 2.71 | 2.87 | 1.25 | 3.33 | 1.94 |
| CR002934 | 7.70 | 6.24 | 6.17 | 4.91 | 10.50 | 8.58 |
| CR002935 | 4.20 | 3.64 | 1.57 | 1.02 | 1.13 | 0.90 |
| CR002936 | 2.17 | 1.55 | 1.73 | 0.86 | 4.47 | 2.97 |
| CR002937 | 5.83 | 4.88 | 4.83 | 3.84 | 4.10 | 2.79 |
| CR002938 | 7.70 | 5.57 | 4.63 | 3.18 | 4.33 | 2.21 |
| CR006092 | 15.05 | 3.61 | 11.90 | 3.68 | 3.25 | 0.07 |
| CR006093 | 33.40 | 16.12 | 33.15 | 15.91 | 0.30 | 0.14 |
| CR006094 | 6.75 | 5.44 | 6.60 | 5.37 | 0.20 | 0.14 |
| CR006095 | 6.55 | 2.90 | 5.95 | 2.47 | 0.60 | 0.42 |
| CR006096 | 1.05 | 1.06 | 1.00 | 0.99 | 0.00 | 0.00 |
| CR006097 | 0.25 | 0.07 | 0.15 | 0.07 | 0.15 | 0.07 |
| CR006098 | 3.40 | 1.98 | 3.10 | 1.84 | 0.40 | 0.14 |
| CR006099 | 4.05 | 0.64 | 3.50 | 0.71 | 0.55 | 0.07 |
| CR006100 | 3.60 | 0.57 | 3.10 | 0.42 | 0.55 | 0.21 |
| CR006101 | 4.60 | 1.27 | 4.35 | 1.20 | 0.30 | 0.00 |
| CR006102 | 4.10 | 2.69 | 3.80 | 2.55 | 0.40 | 0.14 |
| CR006103 | 12.95 | 3.89 | 12.45 | 3.75 | 0.75 | 0.07 |
| CR006104 | 0.85 | 0.07 | 0.75 | 0.07 | 0.05 | 0.07 |
| CR006105 | 3.65 | 1.63 | 3.25 | 1.63 | 0.45 | 0.07 |
| CR006106 | 2.60 | 0.85 | 2.30 | 0.85 | 0.35 | 0.07 |
| CR006107 | 14.55 | 2.62 | 8.10 | 2.26 | 6.50 | 0.28 |
| CR006108 | 6.15 | 2.47 | 5.95 | 2.33 | 0.20 | 0.14 |
| CR006109 | 32.30 | 11.60 | 31.25 | 11.10 | 1.10 | 0.57 |
| CR006110 | 3.70 | 1.13 | 2.95 | 0.78 | 0.75 | 0.35 |
| CR006111 | 1.10 | 0.57 | 1.00 | 0.42 | 0.10 | 0.14 |
| CR006112 | 7.35 | 0.21 | 7.10 | 0.28 | 0.25 | 0.07 |
| CR006113 | 2.85 | 1.06 | 2.70 | 1.27 | 0.20 | 0.14 |
| CR006114 | 23.85 | 8.56 | 21.15 | 7.00 | 2.75 | 1.63 |
| CR006115 | 9.90 | 4.81 | 7.65 | 3.61 | 2.30 | 1.13 |
| CR006116 | 10.35 | 3.04 | 9.40 | 2.69 | 1.05 | 0.35 |
| CR006117 | 6.95 | 1.34 | 6.20 | 0.85 | 0.95 | 0.49 |
| CR006118 | 3.15 | 0.21 | 2.45 | 0.35 | 0.75 | 0.07 |
| CR006119 | 5.35 | 1.06 | 4.85 | 1.20 | 0.50 | 0.14 |
| CR006120 | 7.15 | 0.35 | 6.35 | 0.07 | 0.80 | 0.42 |
| CR006121 | 11.05 | 3.61 | 7.75 | 2.62 | 3.35 | 1.06 |
| CR006122 | 19.20 | 0.00 | 17.65 | 0.49 | 1.55 | 0.49 |
| CR006123 | 9.40 | 2.97 | 8.90 | 3.25 | 0.55 | 0.21 |
| CR006124 | 9.75 | 2.05 | 8.85 | 1.77 | 0.90 | 0.28 |
| CR006125 | 10.60 | 4.10 | 8.40 | 3.68 | 2.35 | 0.35 |
| CR006126 | 25.60 | 6.93 | 24.10 | 6.65 | 1.55 | 0.21 |
| CR006127 | 5.80 | 3.25 | 5.10 | 2.55 | 0.75 | 0.78 |
| CR006128 | 6.00 | 2.83 | 5.65 | 2.76 | 0.45 | 0.07 |
| CR006129 | 15.40 | 8.20 | 10.15 | 7.00 | 5.50 | 1.13 |
| CR006133 | 9.65 | 4.03 | 5.45 | 3.61 | 4.25 | 0.35 |
| CR006134 | 3.80 | 1.56 | 3.80 | 1.56 | 0.00 | 0.00 |
| CR006135 | 6.10 | 1.27 | 4.90 | 1.56 | 1.20 | 0.28 |
| CR006136 | 5.35 | 0.92 | 2.55 | 0.07 | 2.80 | 0.99 |
| CR006137 | 8.60 | 3.82 | 7.80 | 4.10 | 0.95 | 0.35 |
| CR006138 | 16.85 | 4.31 | 16.70 | 4.10 | 0.20 | 0.28 |
| CR006139 | 3.65 | 2.33 | 3.50 | 2.26 | 0.25 | 0.21 |
| CR006140 | 7.30 | 1.13 | 6.95 | 0.92 | 0.35 | 0.21 |
| CR006141 | 5.35 | 1.34 | 4.10 | 0.28 | 1.20 | 0.99 |
| CR006142 | 3.45 | 0.78 | 3.00 | 0.57 | 0.50 | 0.14 |
| CR006143 | 1.50 | 0.85 | 1.40 | 0.71 | 0.15 | 0.07 |
| CR006144 | 2.40 | 0.57 | 2.00 | 0.42 | 0.45 | 0.21 |
| CR006145 | 6.85 | 0.49 | 6.65 | 0.64 | 0.25 | 0.21 |
| CR006146 | 4.45 | 2.47 | 4.05 | 2.62 | 0.45 | 0.07 |
| CR006147 | 2.70 | 1.13 | 2.50 | 0.99 | 0.25 | 0.21 |
| CR006148 | 9.70 | 3.25 | 8.05 | 2.90 | 2.20 | 0.57 |
| CR006149 | 14.20 | 5.80 | 13.60 | 5.94 | 0.65 | 0.07 |
| CR006150 | 11.05 | 6.72 | 9.30 | 6.51 | 2.10 | 0.42 |
| CR006151 | 4.60 | 2.83 | 4.35 | 2.47 | 0.25 | 0.35 |
| CR006152 | 7.35 | 2.90 | 7.20 | 2.83 | 0.15 | 0.07 |
| CR001263 | 8.65 | 3.18 | 8.05 | 2.90 | 0.80 | 0.42 |
| CR006153 | 8.10 | 1.41 | 6.45 | 0.92 | 1.65 | 0.49 |
| CR006154 | 20.30 | 6.08 | 19.40 | 6.22 | 0.95 | 0.21 |
| CR006155 | 10.40 | 2.83 | 9.80 | 2.83 | 0.70 | 0.00 |

Table 10 shows the average and standard deviation for % Edit, % Insertion (Ins), and % Deletion (Del) for the tested HAO1 dgRNAs co-transfected with Spy Cas9 protein in primary cynomolgus hepatocytes. N=3.

TABLE 10
HAO1 editing data for crRNAs delivered
to primary cynomolgus hepatocytes

| GUIDE | Avg | Std Dev | Avg | Std Dev | Avg | Std Dev |
|---|---|---|---|---|---|---|
| ID | % Edit | % Edit | % Ins | % Ins | % Del | % Del |
| CR002857 | 19.18 | 3.25 | 17.01 | 2.91 | 2.18 | 0.57 |
| CR002858 | 9.13 | 1.14 | 7.91 | 1.62 | 1.22 | 0.50 |
| CR002859 | 9.50 | 1.71 | 8.14 | 0.95 | 1.35 | 0.79 |
| CR002860 | 49.63 | 11.68 | 45.38 | 10.65 | 4.25 | 1.03 |
| CR002861 | 23.04 | 1.48 | 19.14 | 0.17 | 3.90 | 1.65 |
| CR002862 | 43.12 | 6.71 | 41.25 | 6.90 | 1.87 | 0.59 |
| CR002863 | 11.28 | 0.75 | 9.63 | 0.65 | 1.65 | 0.39 |
| CR002864 | 16.06 | 2.42 | 15.89 | 2.55 | 0.17 | 0.13 |
| CR002865 | 11.26 | 1.06 | 6.58 | 1.16 | 4.67 | 0.37 |
| CR002866 | 1.33 | 0.38 | 1.18 | 0.31 | 0.15 | 0.07 |
| CR002867 | 3.70 | 0.39 | 2.54 | 0.20 | 1.16 | 0.36 |
| CR002868 | 5.90 | 0.93 | 5.62 | 0.53 | 0.27 | 0.40 |
| CR002869 | 1.07 | 0.25 | 0.92 | 0.20 | 0.15 | 0.05 |
| CR002871 | 14.28 | 3.72 | 14.09 | 3.76 | 0.19 | 0.22 |
| CR002872 | 4.19 | 0.78 | 4.06 | 0.86 | 0.13 | 0.11 |
| CR002873 | 8.86 | 1.50 | 8.28 | 1.34 | 0.57 | 0.18 |
| CR002874 | 7.61 | 0.52 | 7.42 | 0.67 | 0.18 | 0.17 |
| CR002875 | 2.61 | 0.63 | 2.51 | 0.70 | 0.10 | 0.09 |
| CR002876 | 1.82 | 0.73 | 1.62 | 0.75 | 0.20 | 0.03 |
| CR002877 | 2.53 | 0.69 | 2.44 | 0.70 | 0.09 | 0.01 |
| CR002878 | 27.13 | 1.50 | 4.98 | 1.04 | 22.15 | 1.75 |
| CR002879 | 86.74 | 1.87 | 0.04 | 0.03 | 86.70 | 1.89 |
| CR002880 | 47.71 | 0.61 | 0.73 | 0.13 | 46.98 | 0.74 |
| CR002881 | 3.86 | 0.37 | 3.48 | 0.24 | 0.38 | 0.22 |
| CR002882 | 3.52 | 0.89 | 3.33 | 0.84 | 0.19 | 0.15 |
| CR002883 | 6.52 | 1.43 | 6.15 | 1.59 | 0.38 | 0.15 |
| CR002884 | 3.88 | 0.02 | 3.84 | 0.02 | 0.04 | 0.01 |
| CR002885 | 1.39 | 0.60 | 1.36 | 0.57 | 0.02 | 0.03 |
| CR002886 | 4.80 | 0.98 | 4.60 | 1.05 | 0.20 | 0.10 |
| CR002887 | 15.92 | 3.08 | 15.51 | 3.01 | 0.41 | 0.15 |
| CR002888 | 2.70 | 0.53 | 2.60 | 0.55 | 0.10 | 0.04 |
| CR002889 | 1.68 | 0.38 | 1.58 | 0.36 | 0.10 | 0.02 |
| CR002890 | 3.82 | 0.79 | 3.48 | 0.68 | 0.33 | 0.31 |
| CR002891 | 8.19 | 1.69 | 7.98 | 1.39 | 0.21 | 0.30 |
| CR002892 | 1.84 | 0.50 | 1.64 | 0.54 | 0.20 | 0.05 |
| CR002893 | 1.87 | 0.36 | 1.73 | 0.33 | 0.14 | 0.04 |
| CR002894 | 44.20 | 2.89 | 42.46 | 3.43 | 1.74 | 0.54 |
| CR002895 | 8.72 | 1.00 | 4.86 | 1.04 | 3.86 | 0.45 |
| CR002896 | 5.15 | 1.38 | 4.34 | 0.88 | 0.81 | 0.50 |
| CR002897 | 6.12 | 1.95 | 1.74 | 0.31 | 4.38 | 1.65 |
| CR002898 | 1.50 | 0.64 | 1.43 | 0.54 | 0.08 | 0.11 |
| CR002899 | 2.13 | 0.48 | 1.97 | 0.48 | 0.16 | 0.01 |
| CR002900 | 1.98 | 0.66 | 1.81 | 0.73 | 0.17 | 0.08 |
| CR002901 | 5.58 | 1.64 | 5.33 | 1.58 | 0.25 | 0.06 |
| CR002902 | 5.71 | 0.56 | 5.17 | 0.49 | 0.54 | 0.16 |
| CR002903 | 4.89 | 0.56 | 4.43 | 0.42 | 0.47 | 0.14 |
| CR002904 | 9.24 | 1.44 | 8.63 | 1.19 | 0.62 | 0.32 |
| CR002905 | 2.53 | 0.26 | 2.17 | 0.14 | 0.37 | 0.13 |
| CR002906 | 3.22 | 0.21 | 2.64 | 0.07 | 0.57 | 0.24 |
| CR002907 | 2.31 | 0.20 | 2.19 | 0.17 | 0.12 | 0.05 |
| CR002908 | 2.31 | 0.39 | 2.04 | 0.39 | 0.27 | 0.02 |
| CR002909 | 3.01 | 1.06 | 2.82 | 1.05 | 0.19 | 0.12 |
| CR002910 | 3.08 | 0.51 | 2.66 | 0.55 | 0.41 | 0.24 |
| CR002911 | 2.36 | 0.48 | 2.00 | 0.28 | 0.36 | 0.20 |
| CR002912 | 21.23 | 1.26 | 21.08 | 1.26 | 0.15 | 0.04 |
| CR002913 | 1.71 | 0.37 | 1.41 | 0.29 | 0.30 | 0.08 |
| CR002914 | 1.58 | 0.17 | 1.10 | 0.32 | 0.48 | 0.34 |
| CR002916 | 3.14 | 1.76 | 2.07 | 0.84 | 1.07 | 1.01 |
| CR002917 | 2.08 | 0.29 | 1.49 | 0.54 | 0.59 | 0.58 |
| CR002918 | 1.78 | 1.22 | 0.52 | 0.04 | 1.26 | 1.20 |
| CR002919 | 3.30 | 0.26 | 3.23 | 0.25 | 0.07 | 0.04 |
| CR002920 | 1.90 | 0.63 | 1.49 | 0.55 | 0.40 | 0.50 |
| CR002921 | 1.43 | 0.37 | 1.31 | 0.35 | 0.13 | 0.03 |
| CR002928 | 4.59 | 0.84 | 4.27 | 0.70 | 0.32 | 0.14 |
| CR002929 | 41.10 | 4.34 | 40.91 | 4.44 | 0.19 | 0.16 |
| CR002930 | 4.70 | 1.39 | 2.53 | 0.36 | 2.17 | 1.06 |
| CR002931 | 4.60 | 0.52 | 4.54 | 0.53 | 0.06 | 0.02 |
| CR002932 | 6.46 | 0.95 | 4.56 | 0.57 | 1.90 | 0.73 |
| CR002933 | 2.47 | 0.45 | 1.97 | 0.42 | 0.50 | 0.05 |
| CR002934 | 12.77 | 0.88 | 11.45 | 1.05 | 1.32 | 0.24 |
| CR002935 | 3.14 | 0.76 | 2.31 | 0.76 | 0.83 | 0.07 |
| CR002936 | 3.73 | 0.35 | 2.53 | 0.16 | 1.19 | 0.19 |
| CR002937 | 2.99 | 0.26 | 2.77 | 0.19 | 0.22 | 0.08 |
| CR002938 | 7.65 | 0.49 | 6.25 | 0.69 | 1.40 | 0.20 |
| CR006098 | 0.2 | 0.14 | 0.1 | 0 | 0.1 | 0.14 |
| CR006106 | 0.6 | 0.14 | 0.4 | 0 | 0.25 | 0.07 |
| CR006108 | 6.65 | 2.33 | 6.55 | 2.33 | 0.1 | 0 |
| CR006131 | 14.75 | 3.75 | 9.45 | 1.34 | 5.35 | 2.33 |
| CR006132 | 11.6 | 2.83 | 5.9 | 0.57 | 5.65 | 2.33 |
| CR006150 | 5.85 | 1.48 | 5.35 | 1.48 | 0.4 | 0 |
| CR006154 | 8.55 | 4.45 | 8.5 | 4.38 | 0.1 | 0 |
| CR006155 | 2.45 | 1.06 | 2.35 | 1.06 | 0.2 | 0 |

A correlation was calculated by comparing the editing efficiencies for each guide between PHH and each of the cell types (FIG. 1).

Based on the primary human hepatocyte editing data, a subset of guide sequences were further evaluated. This subset is provided in Table 11, with the corresponding editing data from primary human hepatocyte screen reproduced.

TABLE 11
HAO1 editing data for crRNAs in primary human
hepatocytes chosen for further analysis

| GUIDE ID | % Edit |
|---|---|
| CR002860 | 33.40 |
| CR002861 | 23.77 |
| CR002862 | 18.50 |
| CR002864 | 22.47 |
| CR002878 | 19.57 |
| CR002891 | 24.70 |
| CR002894 | 33.80 |
| CR002895 | 15.27 |
| CR002912 | 24.73 |
| CR002929 | 23.60 |
| CR006093 | 33.40 |
| CR006109 | 32.30 |
| CR006114 | 23.85 |
| CR006122 | 19.20 |
| CR006126 | 25.60 |
| CR006138 | 16.85 |
| CR006154 | 20.30 |

### Off Target Analysis of HAO1 Guides

An oligo insertion based assay (See, e.g., Tsai et al., Nature Biotechnology. 33, 187-197; 2015) was used to determine potential off-target genomic sites cleaved by Cas9 targeting HAO1. The 17 dgRNAs in Table 11 (and three control guides with known off-target profiles) were screened in the HEK293-Cas9 cells as described above, and the off-target results were plotted in FIG. 2. The assay identified potential off-target sites for some of the dgRNAs and identified others that had no detectable off-targets. Modified guides that had no or few potential off-target sites identified were synthesized as sgRNA for further analysis (Table 2).

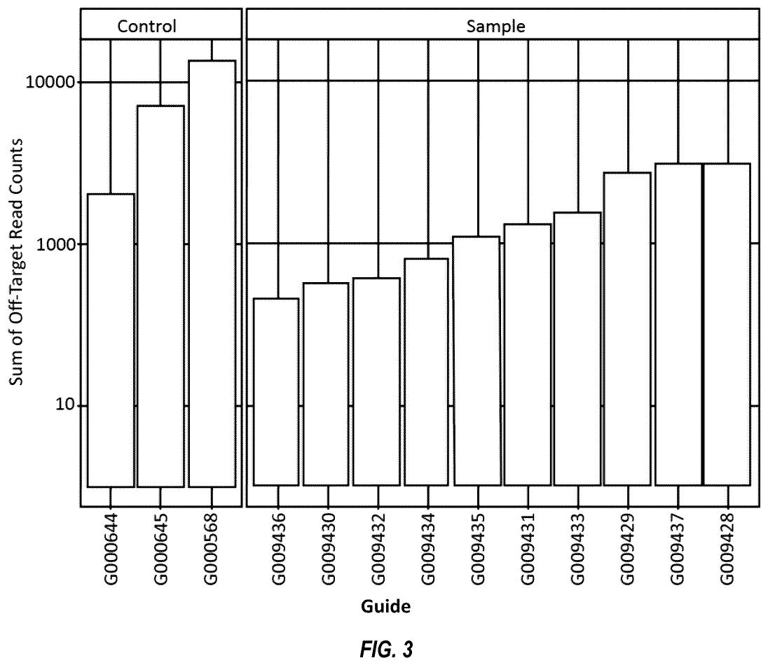

In addition, a biochemical method (See, e.g., Cameron et al., Nature Methods. 6, 600-606; 2017) was used to determine potential off-target genomic sites cleaved by Cas9 targeting HAO1. The 10 modified sgRNA in Table 2 (and three control guides with known off-target profiles) were screened using HEK293 genomic DNA as described above, and the potential off-target results were plotted in FIG. 3. The assay identified potential off-target sites for some of the sgRNAs.

### Targeted Sequencing for Validating Potential Off-Target Sites

The HEK293_Cas9 cells used for detecting potential off-targets constitutively overexpress Cas9, leading to a higher number of potential off-target “hits” as compared to a transient delivery paradigm in various cell types. Further, the biochemical assay typically overrepresents the number of potential off-target sites as the assay utilizes purified high molecular weight genomic DNA free of the cell environment and is dependent on the dose of Cas9 RNP used. Accordingly, potential off-target sites identified by these methods may be validated using targeted sequencing of the identified potential off-target sites.

In one approach, primary hepatocytes are treated with LNPs comprising Cas9 mRNA and a sgRNA of interest (e.g., a sgRNA having potential off-target sites for evaluation). The primary hepatocytes are then lysed and primers flanking the potential off-target site(s) are used to generate an amplicon for NGS analysis. Identification of indels at a certain level may validate potential off-target site, whereas the lack of indels found at the potential off-target site may indicate a false positive in the HEK293_Cas9 cell assay or the biochemical assay.

Cross Screening of Lipid Nanoparticle (LNP) Formulations Containing Spy Cas9 mRNA and sgRNA in Primary Human and Cynomolgus Hepatocytes

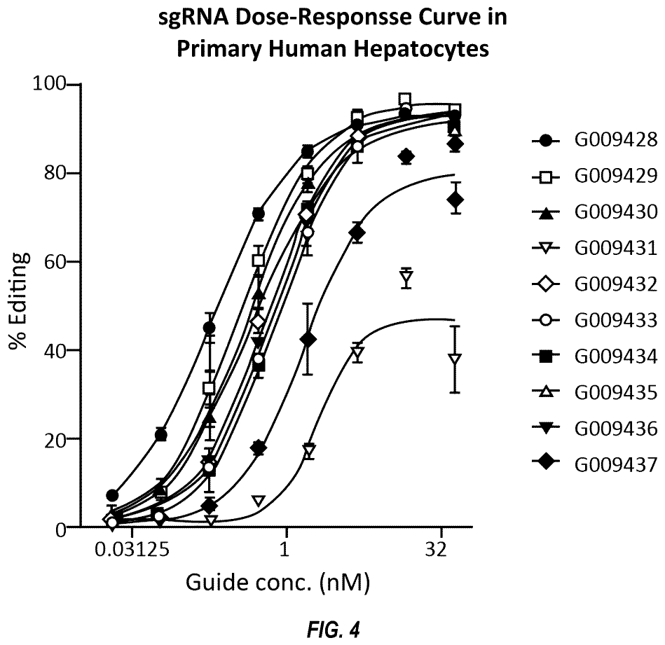

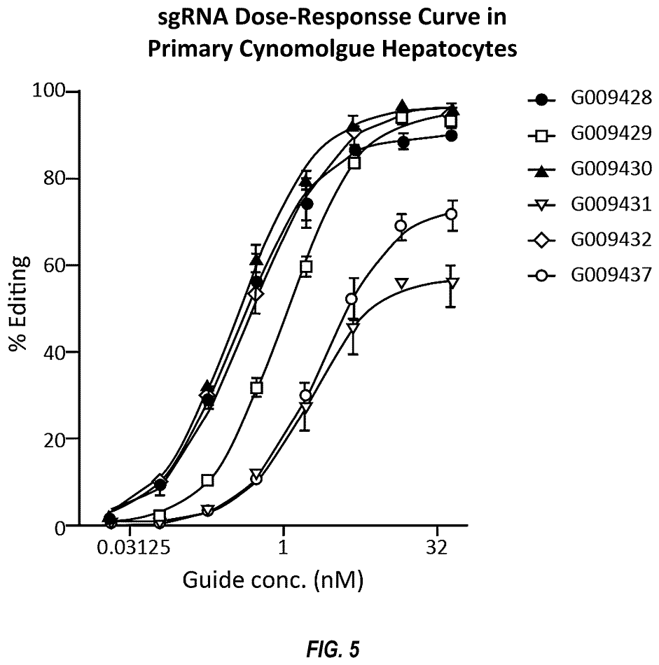

Lipid nanoparticle (LNP) formulations of modified sgRNAs targeting human HAO1 and the cyno matched sgRNA sequences were tested on primary human hepatocytes and primary cynomolgus hepatocytes in a dose response assay. The LNPs were formulated as described in Example 1. Primary human and cynomolgus hepatocytes were plated as described in Example 1. Both cell lines were incubated at 37° C., 5% CO₂ for 48 hours prior to treatment with LNPs. LNPs were incubated in media containing 6% cynomolgus serum at 37° C. for 10 minutes. Post-incubation the LNPs were added to the human or cynomolgus hepatocytes in an 8 point 3-fold dose response curve starting at 300 ng Cas9 mRNA. The cells were lysed 96 hours post-treatment for NGS analysis as described in Example 1. The dose response curve data for the guide sequences in both cell lines is shown in FIGS. 4 and 5. The % editing at the 14.7 nM concentration are listed below in Tables 12 and 13.

Table 12 shows the average and standard deviation for % Edit, % Insertion (Ins), and % Deletion (Del) for the tested HAO1 sgRNAs at 14.7 nM delivered with Spy Cas9 via LNP in primary human hepatocytes. These samples were generated in triplicate.

TABLE 12
HAO1 editing data for sgRNAs expressed
in primary human hepatocytes at 14.7 nM

| GUIDE | Avg | Std Dev | Avg | Std Dev | Avg | Std Dev | |
|---|---|---|---|---|---|---|---|
| ID | % Edit | % Edit | % Ins | % Ins | % Del | % Del | EC50 |
| G009428 | 92.87 | 0.84 | 5.07 | 0.42 | 88.47 | 1.37 | 0.19 |
| G009429 | 96.97 | 0.35 | 0.80 | 0.00 | 96.23 | 0.38 | 0.36 |
| G009430 | 95.60 | 0.75 | 84.90 | 1.56 | 10.77 | 0.95 | 0.43 |
| G009431 | 56.23 | 2.20 | 9.40 | 1.56 | 47.13 | 1.19 | 2.12 |
| G009432 | 93.73 | 0.29 | 62.57 | 3.58 | 32.57 | 2.55 | 0.46 |
| G009433 | 95.67 | 0.61 | 1.37 | 0.15 | 94.30 | 0.61 | 0.79 |
| G009434 | 94.67 | 1.57 | 3.13 | 0.29 | 91.57 | 1.63 | 0.73 |
| G009435 | 95.87 | 0.49 | 15.83 | 1.35 | 80.17 | 1.44 | 0.71 |
| G009436 | 94.83 | 1.29 | 7.67 | 0.25 | 87.40 | 1.28 | 0.72 |
| G009437 | 83.57 | 1.27 | 8.10 | 1.31 | 75.63 | 2.29 | 1.46 |

Table 13 shows the average and standard deviation for % Edit, % Insertion (Ins), and % Deletion (Del) for the tested HAO1 sgRNAs at 14.7 nM delivered with Spy Cas9 via LNP in primary cynomolgus hepatocytes. These samples were generated in triplicate.

TABLE 13
HAO1 editing data for sgRNAs expressed in
primary cynomolgus hepatocytes at 14.7 nM

| GUIDE | Avg | Std Dev | Avg | Std Dev | Avg | Std Dev | |
|---|---|---|---|---|---|---|---|
| ID | % Edit | % Edit | % Ins | % Ins | % Del | % Del | EC50 |
| G009428 | 89.03 | 1.53 | 6.97 | 1.05 | 82.53 | 1.96 | 0.36 |
| G009429 | 94.87 | 1.85 | 3.23 | 0.55 | 92.20 | 1.61 | 1.02 |
| G009430 | 97.33 | 0.84 | 88.07 | 0.67 | 9.33 | 0.81 | 0.35 |
| G009431 | 55.93 | 0.38 | 8.07 | 1.56 | 48.80 | 1.54 | 1.77 |
| G009432 | 96.50 | 0.98 | 84.00 | 1.15 | 12.90 | 1.31 | 0.47 |
| G009437 | 69.10 | 2.62 | 6.80 | 0.66 | 63.20 | 2.82 | 2.30 |

### Example 3. Phenotypic Analysis

### Quantitative PCR Analysis of HAO1 Transcript

Primary human hepatocytes were treated with LNP (as described in Example 1) formulated with the modified sgRNAs from Table 2. The LNPs were formulated as described in Example 1. At twenty-one days post-transfection, cells were harvested and RNA was isolated and subjected to analysis by quantitative PCR as described in Example 1.

RNA was analyzed in triplicate for reduction of HAO1 mRNA, which was calculated using the Ct values determined from the StepOnePlus Real-Time PCR System. A ratio was calculated for the Ct values for 18S within each sample compared to the values for HAO1. Percent reduction of HAO1 mRNA was determined after the ratios were normalized to negative control. The data for % reduction of HAO1 mRNA is provided in Table 14.

TABLE 14
Relative HAO1 mRNA amount in primary
human hepatocytes treated with LNPs

| | | % Remaining | Plus % | Minus % |
|---|---|---|---|---|
| SEQ ID No | GUIDE ID | HAO1 mRNA | Error | Error |
| 251 | G009428 | 37.4 | 0.3 | 0.3 |
| 252 | G009429 | 3.7 | 1.4 | 1.0 |
| 253 | G009430 | 2.3 | 0.6 | 0.4 |
| 254 | G009431 | 7.1 | 1.7 | 1.4 |
| 255 | G009432 | 6.1 | 9.1 | 3.6 |
| 256 | G009433 | 1.8 | 7.6 | 1.5 |
| 257 | G009434 | 4.5 | 1.8 | 1.3 |
| 258 | G009435 | 2.1 | 0.3 | 0.3 |
| 259 | G009436 | 5.6 | 8.3 | 3.4 |
| 260 | G009437 | 33.1 | 3.9 | 3.5 |

### Western Blot Analysis of Intracellular Glycolate Oxidase

A portion of the cells from the quantitative PCR analysis of HAO1 were also harvested twenty-one days post-transfection and whole cell extracts (WCEs) were prepared and subjected to analysis by Western Blot as described in Example 1.

A portion of cells were also collected and processed for NGS sequencing as described herein. The editing data for these cells is provided in Table 15.

TABLE 15
HAO1 editing data for sgRNA delivered to
primary human and cynomolgus hepatocytes

| GUIDE ID | % Edit PHH | % Edit PCH |
|---|---|---|
| G009428 | 94 | 89 |
| G009429 | 95 | 91 |
| G009430 | 93 | 97 |
| G009431 | 72 | 70 |
| G009432 | 91 | 97 |
| G009433 | 93 | N/A |
| G009434 | 93 | N/A |
| G009435 | 97 | N/A |
| G009436 | 95 | N/A |
| G009437 | 91 | 99 |

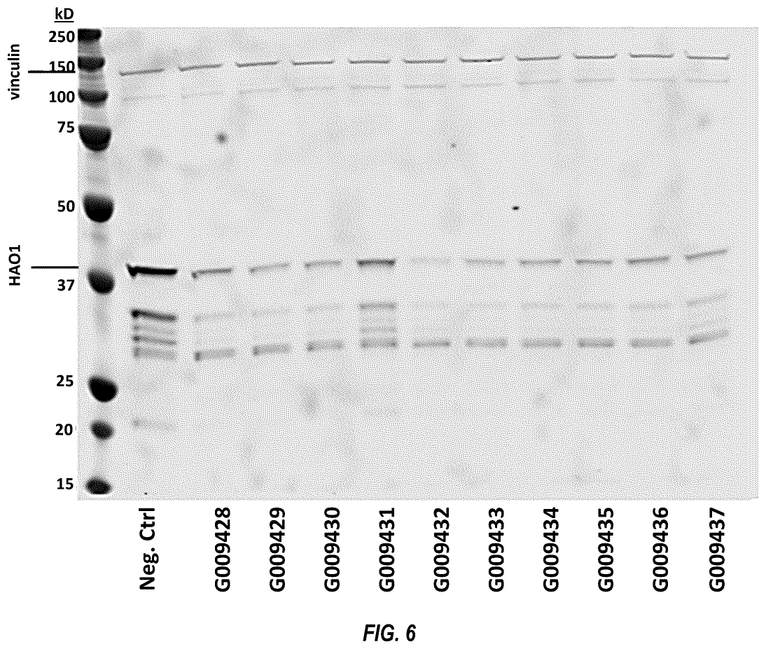

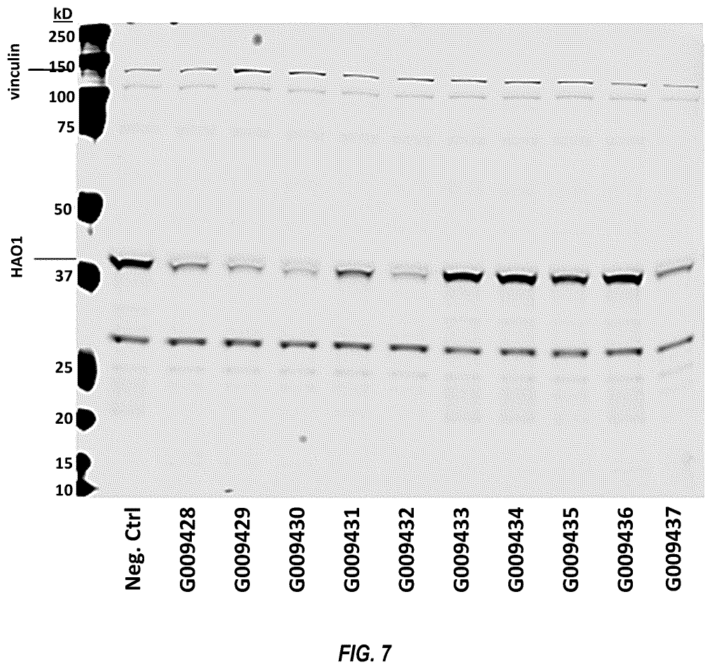

WCEs were analyzed by Western Blot for reduction of GO protein. Full length GO protein has 370 amino acids and a predicted molecular weight of 41 kD. A band at this molecular weight was observed in the control lanes (untreated cells) in the Western Blots (FIGS. 6 and 7).

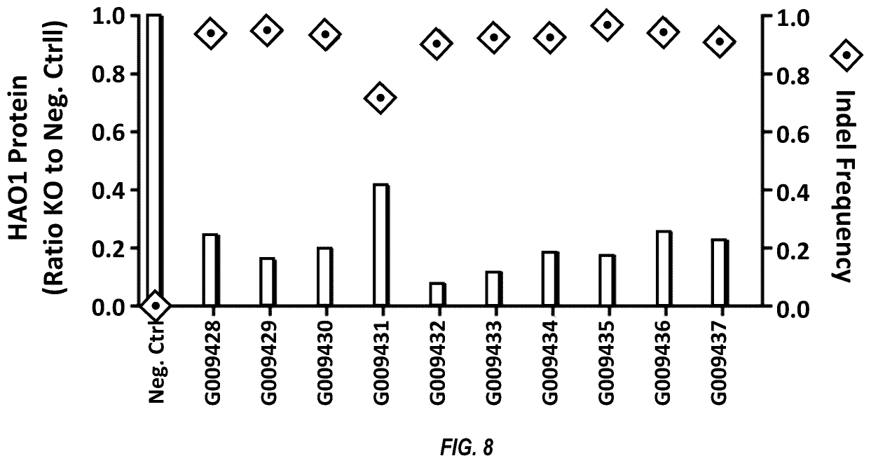

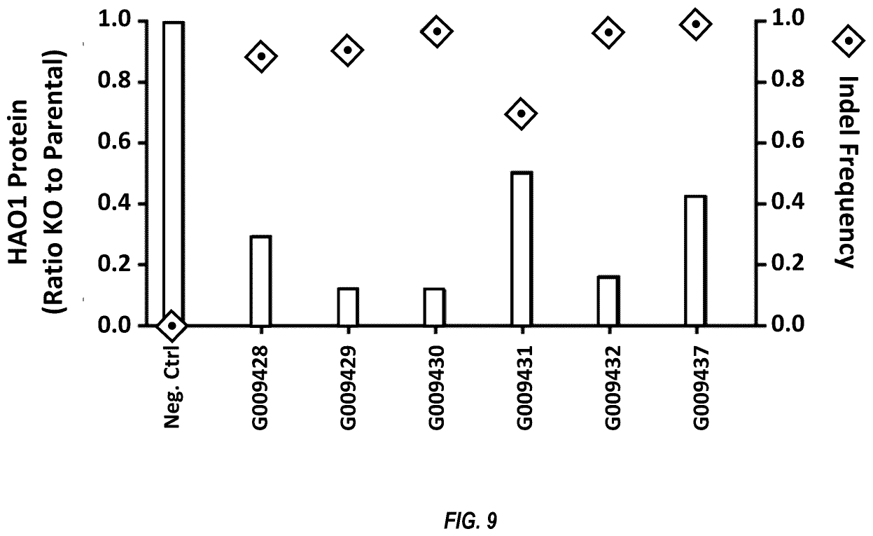

Percent reduction of GO protein was calculated using the Licor Odyssey Image Studio Ver 5.2 software. Vinculin was used as a loading control and probed simultaneously with GO. A ratio was calculated for the densitometry values for vinculin within each sample compared to the total region encompassing the band for GO. Percent reduction of GO protein was determined after the ratios were normalized to negative control lanes. Results are shown in Table 16 and depicted in FIGS. 8 and 9.

TABLE 16
Relative GO protein remaining in primary human
and cynomolgus hepatocytes treated with sgRNAs

| | Protein remaining (relative | Protein remaining (relative |
|---|---|---|
| GUIDE ID | to negative control) in PHH | to negative control) in PCH |
| G009428 | 0.25 | 0.30 |
| G009429 | 0.16 | 0.13 |
| G009430 | 0.20 | 0.12 |
| G009431 | 0.42 | 0.51 |
| G009432 | 0.8 | 0.17 |
| G009433 | 0.12 | N/A |
| G009434 | 0.19 | N/A |
| G009435 | 0.18 | N/A |
| G009436 | 0.26 | N/A |
| G009437 | 0.23 | 0.43 |

### Example 4. In Vivo Editing of Hao1 in a Mouse Model of PH1

Both wildtype and AGT-deficient mice (Agxt1^{-/-}), e.g., null mutant mice lacking liver AGXT mRNA and protein were used in this study. The AGT-deficient mice exhibit hyperoxaluria and crystalluria and thus represent a phenotypic model of PH1, as previously described by Salido et al., Proc Natl Acad Sci USA. 2006 Nov. 28; 103(48):18249-54. The wildtype mice were used to determine which formulation to test in the AGT-deficient mice.

Prior to formulating LNPs, dgRNAs targeting murine Hao1 were screened for editing efficiency similarly as described in Example 2 for the human and cyno HAO1-targeting gRNAs. Having identified active dgRNAs, a smaller set of modified sgRNAs were synthesized for further evaluation in vivo.

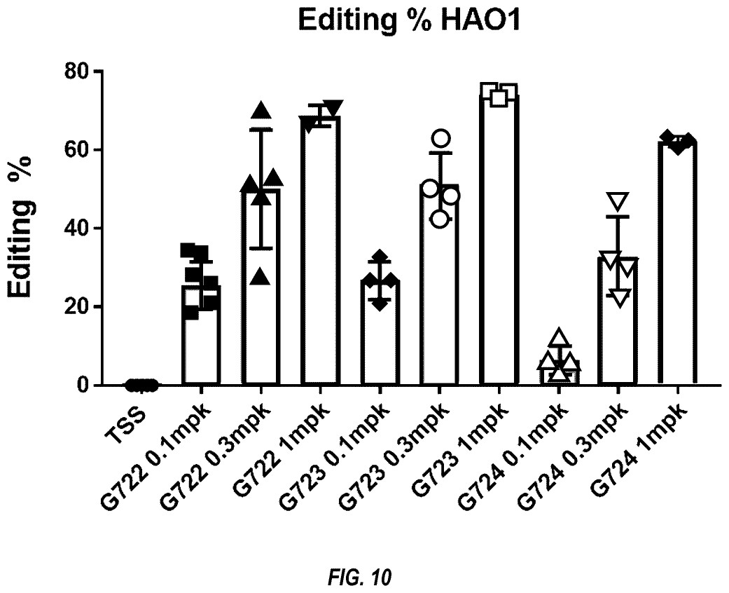

Animals were weighed and grouped according to body weight for preparing dosing solutions based on group average weight. LNPs containing modified sgRNAs targeting murine Hao1 (see Table 17 below) were dosed via the lateral tail vein in a volume of 0.2 mL per animal (approximately 10 mL per kilogram body weight). The LNPs were formulated as described in Example 1. One week post-treatment, wildtype mice were euthanized and liver tissue was collected for DNA extraction and analysis of editing of murine Hao1. As shown in FIG. 10 and Table 17 below, dose-dependent levels of editing were observed in treated mice.

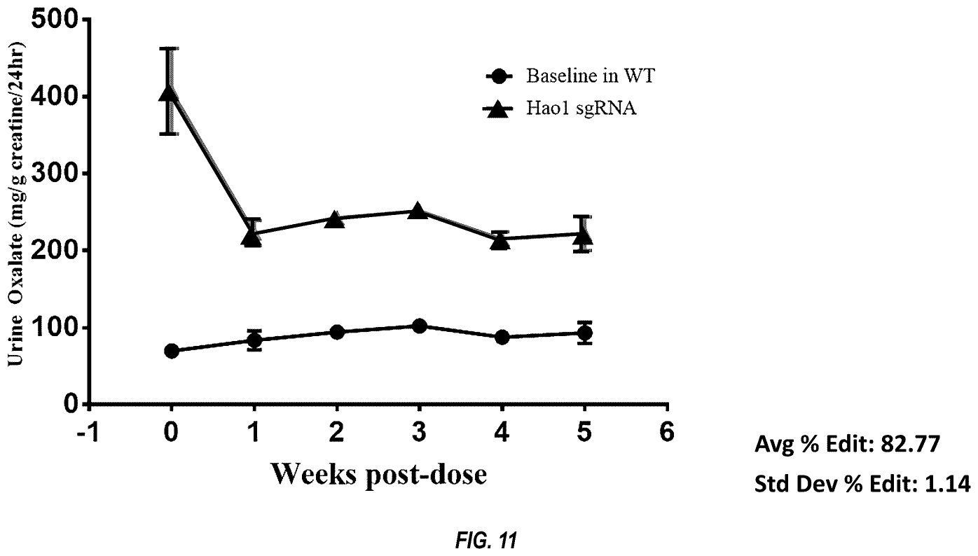

Having established the LNPs could edit the mouse Hao1 gene in vivo, LNP containing G723 was administered to the AGT-deficient mice at a dose of 2 mpk (n=4). These mice were housed in metabolic cages and urine was collected at various time points for oxalate levels, e.g., as described by Liebow et al., J Am Soc Nephrol. 2017 February; 28(2):494-503. Table 18 shows editing results for the AGT-deficient mice. The average % editing achieved (n=4) was 79.85, std. dev. 5.91. As shown in FIG. 11, urine oxalate levels were reduced one week following treatment and this level of reduction was sustained out to at least 5 weeks post-dose at which point the study was terminated. The data depicted in FIG. 11 are shown in Table 19. No reduction was observed (data not shown) in control (PBS injected) animals (n=3).

TABLE 17
Wildtype mouse model editing

| Guide | | Dose | Avg % | Std Dev | n |
|---|---|---|---|---|---|
| ID | sgRNA Sequence | (mpk) | Edit | % Edit | |
| G000722 | mU*mC*mA*CUGAUGCAGACCAGUCGG | 0.1 | 26.82 | 5.62 | 5 |
| | UUUUAGAmGmCmUmAmGmAmAmAmU | 0.3 | 45.90 | 11.83 | 4 |
| | mAmGmCAAGUUAAAAUAAGGCUAGUC | | | | |
| | CGUUAUCAmAmCmUmUmGmAmAmAmA | 1 | 71.07 | 3.37 | 3 |
| | mAmGmUmGmGmCmAmCmCmGmAmGm | | | | |
| | UmCmGmGmUmGmCmU*mU*mU*mU | | | | |
| | (SEQ ID NO: 169) | | | | |
| G000723 | mC*mA*mC*GUGAGCCAUGCACUGCAG | 0.1 | 27.23 | 5.40 | 4 |
| | UUUUAGAmGmCmUmAmGmAmAmAmU | 0.3 | 51.13 | 8.12 | 4 |
| | mAmGmCAAGUUAAAAUAAGGCUAGUC | | | | |
| | CGUUAUCAmAmCmUmUmGmAmAmAmA | 1 | 74.43 | 0.93 | 3 |
| | mAmGmUmGmGmCmAmCmCmGmAmGm | | | | |
| | UmCmGmGmUmGmCmU*mU*mU*mU | | | | |
| | (SEQ ID NO: 170) | | | | |
| G000724 | mU*mC*mU*UUUCUUACCUCGCACAGG | 0.1 | 7.30 | 4.06 | 4 |
| | UUUUAGAmGmCmUmAmGmAmAmAmU | 0.3 | 34.23 | 10.30 | 4 |
| | mAmGmCAAGUUAAAAUAAGGCUAGUC | | | | |
| | CGUUAUCAmAmCmUmUmGmAmAmAmA | 1 | 63.50 | 2.23 | 3 |
| | mAmGmUmGmGmCmAmCmCmGmAmGm | | | | |
| | UmCmGmGmUmGmCmU*mU*mU*mU | | | | |
| | (SEQ ID NO: 171) | | | | |

* = PS linkage; ‘m’ = 2′-O-Me nucleotide

TABLE 18
Agxt1^{-/-} Mouse Model Editing Data, 5 Week Study

| Mouse # | % Edit | % Insertion | % Deletion |
|---|---|---|---|
| 1 | 71.1 | 47.7 | 23.4 |
| 2 | 83.1 | 56 | 27.1 |
| 3 | 81.5 | 52.8 | 28.7 |
| 4 | 83.7 | 54.9 | 28.8 |

TABLE 19
Agxt1^{-/-} Mouse Model Average Urine Oxalate

| | Avg Urine Oxalate | Std Dev Avg |
|---|---|---|
| Week | (mg/g creatinine/24 hr) | Urine Oxalate |
| 0 | 407.00 | 55.70 |
| 1 | 222.75 | 16.90 |
| 2 | 243.00 | 6.38 |
| 3 | 251.75 | 3.86 |
| 4 | 215.00 | 9.70 |
| 5 | 222.00 | 22.11 |

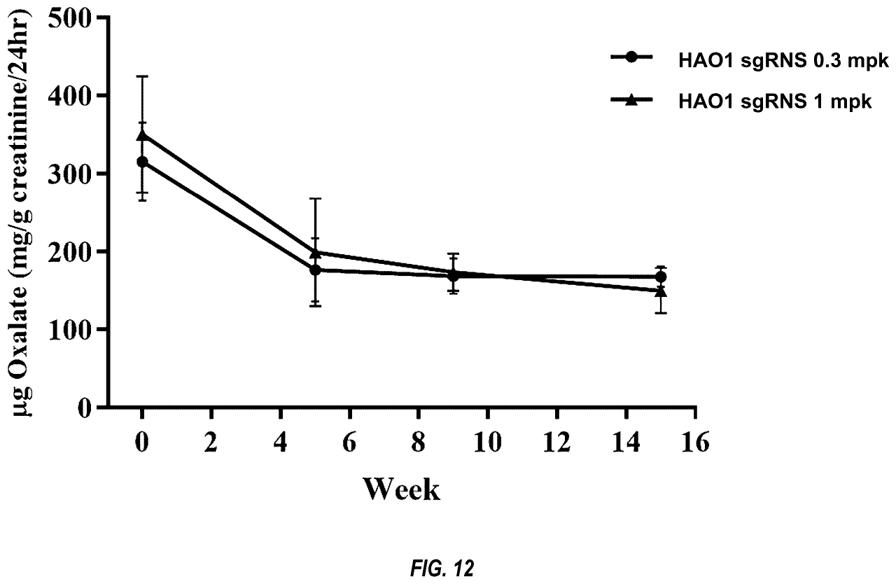

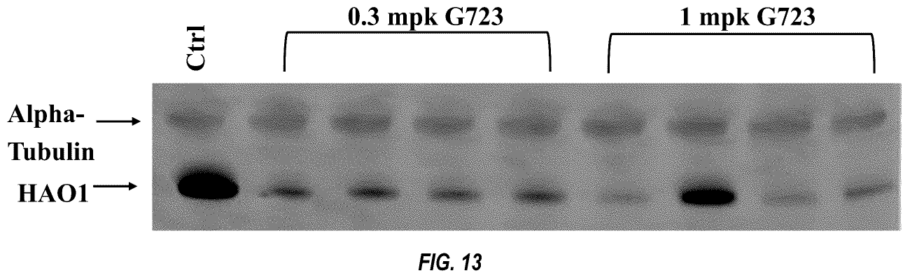

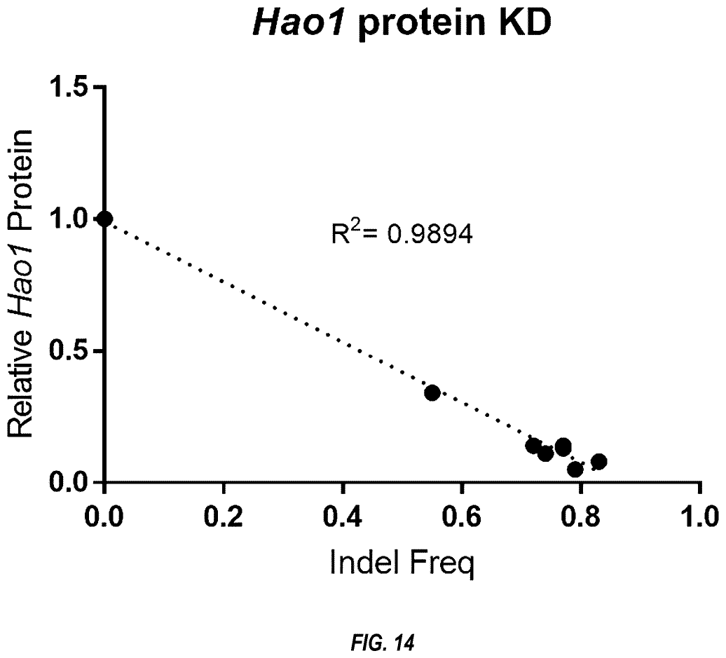

Having demonstrated sustained urine oxalate reduction in AGT-deficient mice up to 5 weeks after LNP treatment, an additional study was conducted to track urine oxalate up to 15 weeks post-dose. LNP containing G723 was administered to AGT-deficient mice at doses of 0.3 mpk (n=4) and 1 mpk (n=4). These mice were housed in metabolic cages and urine was collected at various time points for oxalate levels, as described above. Table 20 shows the editing results for the AGT-deficient mice in the 15 week study. The average % editing achieved at 0.3 mpk dose was 75.8, std. dev. 2.6. The average % editing achieved at 1 mpk dose was 72.75, std. dev. 12.50. As shown in FIG. 12, urine oxalate levels were reduced following treatment and this level of reduction was sustained to at least 15 weeks post-dose at which point the study was terminated. The data depicted in FIG. 12 are shown in Table 21. No reduction was observed (data not shown) in control (PBS injected) animals (n=3). Liver samples from the treated mice were processed and run on Western Blots as described in Example 1. Percent reduction of GO protein was calculated using the Licor Odyssey Image Studio Ver 5.2 software as described above and is displayed in Table 20. The Western Blot image is displayed in FIG. 13. FIG. 14 shows the correlation with an R² value of 0.99 between the editing and protein levels depicted in Table 20.

TABLE 20
Agxt1^{-/-} Mouse Model Editing and Protein Data, 15 Week Study

| | | | | | GO Protein |
|---|---|---|---|---|---|
| | mpk | % | % | % | remaining (relative |
| Mouse # | G723 | Edit | Insertion | Deletion | to negative control) |
| 1 | 0.3 | 77.3 | 53.8 | 23.5 | 0.14 |
| 2 | 0.3 | 77.0 | 55.1 | 21.9 | 0.14 |
| 3 | 0.3 | 77.0 | 55.7 | 21.3 | 0.13 |
| 4 | 0.3 | 71.9 | 50.1 | 21.8 | 0.14 |
| 5 | 1 | 79.2 | 54.0 | 25.3 | 0.05 |
| 6 | 1 | 54.9 | 41.9 | 13.1 | 0.34 |
| 7 | 1 | 83.1 | 51.7 | 31.4 | 0.08 |
| 8 | 1 | 73.8 | 57.1 | 16.7 | 0.11 |

TABLE 21
Agxt1^{-/-} Mouse Model Average Urine Oxalate

| | | Avg Urine Oxalate | Std Dev Avg |
|---|---|---|---|
| Week | Dose G723 (mpk) | (mg/g creatinine/24 hr) | Urine Oxalate |
| 0 | Control | 377.47 | 58.22 |
| 5 | Control | 413.72 | 77.33 |
| 9 | Control | 354.77 | 43.75 |
| 15 | Control | 345.95 | 88.18 |
| 0 | 0.3 | 315.80 | 50.30 |
| 5 | 0.3 | 176.96 | 40.47 |
| 9 | 0.3 | 169.09 | 22.82 |
| 15 | 0.3 | 168.01 | 13.00 |
| 0 | 1.0 | 350.74 | 74.90 |
| 5 | 1.0 | 199.25 | 68.81 |
| 9 | 1.0 | 173.92 | 23.80 |
| 15 | 1.0 | 150.35 | 29.26 |

1.-2. (canceled)

3. A method of treating primary hyperoxaluria type 1 (PH1), the method comprising administering a composition to a subject in need thereof, wherein the composition comprises:
a guide RNA comprising a guide sequence, wherein the guide sequence
is selected from any one of SEQ ID NOs:1-146; or
comprises at least 17, 18, 19, or 20 contiguous nucleotides of a sequence selected from any one of SEQ ID NOs:1-146; or
is at least 90% identical to a sequence selected from any one of SEQ ID NOs:1-146; or
comprises any one of SEQ ID NOs: 4, 5, 6, 8, 22, 35, 38, 39, 56, 73, 84, 100, 105, 113, 117, 129, 145; or
comprises any one of SEQ ID NOs: 8, 22, 35, 39, 73, 84, 100, 105, 113, 145,
thereby treating the PH1.

4.-7. (canceled)

8. The method of claim 3, wherein the method further comprises administering an RNA-guided DNA binding agent or a nucleic acid encoding an RNA-guided DNA binding agent.

9. A composition comprising:
a guide RNA comprising a guide sequence, wherein the guide sequence
is selected from any one of SEQ ID NOs:1-146; or
comprises at least 17, 18, 19, or 20 contiguous nucleotides of a sequence selected from any one of SEQ ID NOs:1-146; or
is at least 90% identical to a sequence selected from any one of SEQ ID NOs:1-146; or
comprises any one of SEQ ID NOs: 4, 5, 6, 8, 22, 35, 38, 39, 56, 73, 84, 100, 105, 113, 117, 129, 145; or
comprises any one of SEQ ID NOs: 8, 22, 35, 39, 73, 84, 100, 105, 113, 145.

10. The composition of claim 9, wherein the guide RNA is a short-single guide RNA (short-sgRNA), comprising:
the guide sequence;
and
a conserved portion of an sgRNA comprising a hairpin region, wherein the hairpin region lacks at least 5-10 nucleotides.

11.-52. (canceled)

53. The composition of claim 9, wherein the guide sequence constitutes a first guide sequence that is complementary to a first target sequence in the positive strand of the HAO1 gene and the composition further comprises a second guide sequence that is complementary to a second target sequence in the negative strand of the HAO1 gene.

54. The composition of claim 9, wherein the guide RNA comprises a guide sequence selected from any one of SEQ ID NOs:1-146 and further comprises a nucleotide sequence of SEQ ID NO: 200, wherein the nucleotides of SEQ ID NO: 200 follow the guide sequence at its 3′ end.

55. (canceled)

56. The composition of claim 9, wherein the guide RNA is a single guide (sgRNA).

57. The composition of claim 56, wherein the sgRNA comprises a guide sequence comprising any one of SEQ ID NOs: 4, 5, 6, 8, 22, 35, 38, 39, 56, 73, 84, 100, 105, 113, 117, 129, or 145 or the sgRNA comprises any one of SEQ ID NOs: 151-168 or 251-268.

58. (canceled)

59. The composition of claim 9, wherein the guide RNA is modified according to the pattern of SEQ ID NO: 300, wherein the Ns are collectively any one of SEQ ID NOs: 1-146.

60.-63. (canceled)

64. The composition of claim 9, wherein the guide RNA comprises at least one modification, wherein the modification is one or more of a 2′-O-methyl (2′-O-Me)-modified nucleotide; a phosphorothioate bond between nucleotides; and a 2′-fluoro (2′-F) modified nucleotide.

65.-75. (canceled)

76. The composition of claim 9, wherein the guide RNA is associated with a lipid nanoparticle (LNP) comprising a cationic lipid.

77. (canceled)

78. The composition of claim 76, wherein the cationic lipid is (9Z,12Z)-3-((4,4-bis(octyloxy)butanoyl)oxy)-2-((((3-(diethylamino)propoxy)carbonyl)oxy)methyl)propyl octadeca-9,12-dienoate.

79. The composition of claim 76, wherein the LNP further comprises a neutral lipid.

80. The composition of claim 79, wherein the neutral lipid is DSPC.

81. The composition of claim 76, wherein the LNP further comprises a helper lipid.

82. The composition of claim 81, wherein the helper lipid is cholesterol.

83. The composition of claim 76, wherein the LNP further comprises a stealth lipid.

84. The composition of claim 83, wherein the stealth lipid is PEG2k-DMG.

85. The composition of claim 9, wherein the composition further comprises an RNA-guided DNA binding agent.

86. (canceled)

87. The composition of claim 85, wherein the RNA-guided DNA binding agent is Cas9.

88.-264. (canceled)


SEQUENCE LISTING (602 sequences)

SEQ ID NO: 1 - RNA, 20 nt, synthetic construct
  source 1..20: mol_type = other RNA
taatagtcatatatagactt

SEQ ID NO: 2 - RNA, 20 nt, synthetic construct
  source 1..20: mol_type = other RNA
aatcattgatacaaattagc

SEQ ID NO: 3 - RNA, 20 nt, synthetic construct
  source 1..20: mol_type = other RNA
atcattgatacaaattagcc

SEQ ID NO: 4 - RNA, 20 nt, synthetic construct
  source 1..20: mol_type = other RNA
tcattgatacaaattagccg

SEQ ID NO: 5 - RNA, 20 nt, synthetic construct
  source 1..20: mol_type = other RNA
cattgatacaaattagccgg

SEQ ID NO: 6 - RNA, 20 nt, synthetic construct
  source 1..20: mol_type = other RNA
agccgggggagcattttcac

SEQ ID NO: 7 - RNA, 20 nt, synthetic construct
  source 1..20: mol_type = other RNA
ggcaaatgatgaagaaactt

SEQ ID NO: 8 - RNA, 20 nt, synthetic construct
  source 1..20: mol_type = other RNA
aacctgtgaaaatgctcccc

SEQ ID NO: 9 - RNA, 20 nt, synthetic construct
  source 1..20: mol_type = other RNA
cacatgagccatgcgctgca

SEQ ID NO: 10 - RNA, 20 nt, synthetic construct
  source 1..20: mol_type = other RNA
gccgtagcccccacacatat

SEQ ID NO: 11 - RNA, 20 nt, synthetic construct
  source 1..20: mol_type = other RNA
gatctgtttcagcaacattc

SEQ ID NO: 12 - RNA, 20 nt, synthetic construct
  source 1..20: mol_type = other RNA
gcaacattccggagcatcct

SEQ ID NO: 13 - RNA, 20 nt, synthetic construct
  source 1..20: mol_type = other RNA
catgcagcgcatggctcatg

SEQ ID NO: 14 - RNA, 20 nt, synthetic construct
  source 1..20: mol_type = other RNA
gatctgtcgacttctgtttt

SEQ ID NO: 15 - RNA, 20 nt, synthetic construct
  source 1..20: mol_type = other RNA
agctgtatccaaggatgctc

SEQ ID NO: 16 - RNA, 20 nt, synthetic construct
  source 1..20: mol_type = other RNA
ctagatggaagctgtatcca

SEQ ID NO: 17 - RNA, 20 nt, synthetic construct
  source 1..20: mol_type = other RNA
tgtgtccactgtcacaaata

SEQ ID NO: 18 - RNA, 20 nt, synthetic construct
  source 1..20: mol_type = other RNA
cgccacttcttcaattgagg

SEQ ID NO: 19 - RNA, 20 nt, synthetic construct
  source 1..20: mol_type = other RNA
cacttcttcaattgaggagg

SEQ ID NO: 20 - RNA, 20 nt, synthetic construct
  source 1..20: mol_type = other RNA
tcaacatcatgcccgttccc

SEQ ID NO: 21 - RNA, 20 nt, synthetic construct
  source 1..20: mol_type = other RNA
caacatcatgcccgttccca

SEQ ID NO: 22 - RNA, 20 nt, synthetic construct
  source 1..20: mol_type = other RNA
gcccgttcccagggactgac

SEQ ID NO: 23 - RNA, 20 nt, synthetic construct
  source 1..20: mol_type = other RNA
acagtggacacaccttacct

SEQ ID NO: 24 - RNA, 20 nt, synthetic construct
  source 1..20: mol_type = other RNA
gacagtggacacaccttacc

SEQ ID NO: 25 - RNA, 20 nt, synthetic construct
  source 1..20: mol_type = other RNA
caaggccatatttgtgacag

SEQ ID NO: 26 - RNA, 20 nt, synthetic construct
  source 1..20: mol_type = other RNA
ctggtcctgaggcacttcgt

SEQ ID NO: 27 - RNA, 20 nt, synthetic construct
  source 1..20: mol_type = other RNA
cacctcctcaattgaagaag

SEQ ID NO: 28 - RNA, 20 nt, synthetic construct
  source 1..20: mol_type = other RNA
gggcatgatgttgagttcct

SEQ ID NO: 29 - RNA, 20 nt, synthetic construct
  source 1..20: mol_type = other RNA
cgggcatgatgttgagttcc

SEQ ID NO: 30 - RNA, 20 nt, synthetic construct
  source 1..20: mol_type = other RNA
gcctgtcagtccctgggaac

SEQ ID NO: 31 - RNA, 20 nt, synthetic construct
  source 1..20: mol_type = other RNA
agcctgtcagtccctgggaa

SEQ ID NO: 32 - RNA, 20 nt, synthetic construct
  source 1..20: mol_type = other RNA
ttctcagcctgtcagtccct

SEQ ID NO: 33 - RNA, 20 nt, synthetic construct
  source 1..20: mol_type = other RNA
tttctcagcctgtcagtccc

SEQ ID NO: 34 - RNA, 20 nt, synthetic construct
  source 1..20: mol_type = other RNA
aaaatgccctttgcaacaat

SEQ ID NO: 35 - RNA, 20 nt, synthetic construct
  source 1..20: mol_type = other RNA
aggagaaaatgataaagtac

SEQ ID NO: 36 - RNA, 20 nt, synthetic construct
  source 1..20: mol_type = other RNA
tcattgccaattgttgcaaa

SEQ ID NO: 37 - RNA, 20 nt, synthetic construct
  source 1..20: mol_type = other RNA
atcattgccaattgttgcaa

SEQ ID NO: 38 - RNA, 20 nt, synthetic construct
  source 1..20: mol_type = other RNA
tcagctgggaagatatcaaa

SEQ ID NO: 39 - RNA, 20 nt, synthetic construct
  source 1..20: mol_type = other RNA
aatagacccatctatcagct

SEQ ID NO: 40 - RNA, 20 nt, synthetic construct
  source 1..20: mol_type = other RNA
cagtggacttgctgcatatg

SEQ ID NO: 41 - RNA, 20 nt, synthetic construct
  source 1..20: mol_type = other RNA
ttttctcctgaggaaaattt

SEQ ID NO: 42 - RNA, 20 nt, synthetic construct
  source 1..20: mol_type = other RNA
tactttatcattttctcctg

SEQ ID NO: 43 - RNA, 20 nt, synthetic construct
  source 1..20: mol_type = other RNA
ctgccaaaactcacagtggc

SEQ ID NO: 44 - RNA, 20 nt, synthetic construct
  source 1..20: mol_type = other RNA
gccatgtttaacagcctccc

SEQ ID NO: 45 - RNA, 20 nt, synthetic construct
  source 1..20: mol_type = other RNA
tggggctcgacaactcgatg

SEQ ID NO: 46 - RNA, 20 nt, synthetic construct
  source 1..20: mol_type = other RNA
atggggctcgacaactcgat

SEQ ID NO: 47 - RNA, 20 nt, synthetic construct
  source 1..20: mol_type = other RNA
catggggctcgacaactcga

SEQ ID NO: 48 - RNA, 20 nt, synthetic construct
  source 1..20: mol_type = other RNA
gatcttggtgtcgaatcatg

SEQ ID NO: 49 - RNA, 20 nt, synthetic construct
  source 1..20: mol_type = other RNA
ggatcttggtgtcgaatcat

SEQ ID NO: 50 - RNA, 20 nt, synthetic construct
  source 1..20: mol_type = other RNA
gggatcttggtgtcgaatca

SEQ ID NO: 51 - RNA, 20 nt, synthetic construct
  source 1..20: mol_type = other RNA
acatggcttgaatgggatct

SEQ ID NO: 52 - RNA, 20 nt, synthetic construct
  source 1..20: mol_type = other RNA
ctgttaaacatggcttgaat

SEQ ID NO: 53 - RNA, 20 nt, synthetic construct
  source 1..20: mol_type = other RNA
gctgttaaacatggcttgaa

SEQ ID NO: 54 - RNA, 20 nt, synthetic construct
  source 1..20: mol_type = other RNA
gccagggaggctgttaaaca

SEQ ID NO: 55 - RNA, 20 nt, synthetic construct
  source 1..20: mol_type = other RNA
tccaggtgatgatgccaggg

SEQ ID NO: 56 - RNA, 20 nt, synthetic construct
  source 1..20: mol_type = other RNA
ctctccaggtgatgatgcca

SEQ ID NO: 57 - RNA, 20 nt, synthetic construct
  source 1..20: mol_type = other RNA
tctctccaggtgatgatgcc

SEQ ID NO: 58 - RNA, 20 nt, synthetic construct
  source 1..20: mol_type = other RNA
tctccccacaaacacagcct

SEQ ID NO: 59 - RNA, 20 nt, synthetic construct
  source 1..20: mol_type = other RNA
ctttccgcacacccccgtcc

SEQ ID NO: 60 - RNA, 20 nt, synthetic construct
  source 1..20: mol_type = other RNA
gcgccaaggctgtgtttgtg

SEQ ID NO: 61 - RNA, 20 nt, synthetic construct
  source 1..20: mol_type = other RNA
ggcgccaaggctgtgtttgt

SEQ ID NO: 62 - RNA, 20 nt, synthetic construct
  source 1..20: mol_type = other RNA
tggcgccaaggctgtgtttg

SEQ ID NO: 63 - RNA, 20 nt, synthetic construct
  source 1..20: mol_type = other RNA
agctctggctcttggcgcca

SEQ ID NO: 64 - RNA, 20 nt, synthetic construct
  source 1..20: mol_type = other RNA
gttctgaaagctctggctct

SEQ ID NO: 65 - RNA, 20 nt, synthetic construct
  source 1..20: mol_type = other RNA
cactgatgttctgaaagctc

SEQ ID NO: 66 - RNA, 20 nt, synthetic construct
  source 1..20: mol_type = other RNA
ctggacgggggtgtgcggaa

SEQ ID NO: 67 - RNA, 20 nt, synthetic construct
  source 1..20: mol_type = other RNA
tcttcctggacgggggtgtg

SEQ ID NO: 68 - RNA, 20 nt, synthetic construct
  source 1..20: mol_type = other RNA
gtggaagtcttcctggacgg

SEQ ID NO: 69 - RNA, 20 nt, synthetic construct
  source 1..20: mol_type = other RNA
ggtggaagtcttcctggacg

SEQ ID NO: 70 - RNA, 20 nt, synthetic construct
  source 1..20: mol_type = other RNA
aggtggaagtcttcctggac

SEQ ID NO: 71 - RNA, 20 nt, synthetic construct
  source 1..20: mol_type = other RNA
aaggtggaagtcttcctgga

SEQ ID NO: 72 - RNA, 20 nt, synthetic construct
  source 1..20: mol_type = other RNA
agggaaggtggaagtcttcc

SEQ ID NO: 73 - RNA, 20 nt, synthetic construct
  source 1..20: mol_type = other RNA
tgttctgccagaaattgtgg

SEQ ID NO: 74 - RNA, 20 nt, synthetic construct
  source 1..20: mol_type = other RNA
tgatgttctgccagaaattg

SEQ ID NO: 75 - RNA, 20 nt, synthetic construct
  source 1..20: mol_type = other RNA
ggaagaattccggttggcca

SEQ ID NO: 76 - RNA, 20 nt, synthetic construct
  source 1..20: mol_type = other RNA
agatactaaaggaagaattc

SEQ ID NO: 77 - RNA, 20 nt, synthetic construct
  source 1..20: mol_type = other RNA
tcacttggttagggggagaa

SEQ ID NO: 78 - RNA, 20 nt, synthetic construct
  source 1..20: mol_type = other RNA
gcactgtcagatcttggaaa

SEQ ID NO: 79 - RNA, 20 nt, synthetic construct
  source 1..20: mol_type = other RNA
cagatcttggaaacggccaa

SEQ ID NO: 80 - RNA, 20 nt, synthetic construct
  source 1..20: mol_type = other RNA
tgtcgatgactttcacattc

SEQ ID NO: 81 - RNA, 20 nt, synthetic construct
  source 1..20: mol_type = other RNA
tcatcgacaagacattggtg

SEQ ID NO: 82 - RNA, 20 nt, synthetic construct
  source 1..20: mol_type = other RNA
gaaagtcatcgacaagacat

SEQ ID NO: 83 - RNA, 20 nt, synthetic construct
  source 1..20: mol_type = other RNA
agtctatatatgactattac

SEQ ID NO: 84 - RNA, 20 nt, synthetic construct
  source 1..20: mol_type = other RNA
atatatgactattacaggtc

SEQ ID NO: 85 - RNA, 20 nt, synthetic construct
  source 1..20: mol_type = other RNA
tatatgactattacaggtct

SEQ ID NO: 86 - RNA, 20 nt, synthetic construct
  source 1..20: mol_type = other RNA
atatgactattacaggtctg

SEQ ID NO: 87 - RNA, 20 nt, synthetic construct
  source 1..20: mol_type = other RNA
aaaaaataaattttcttacc

SEQ ID NO: 88 - RNA, 20 nt, synthetic construct
  source 1..20: mol_type = other RNA
ttttattttttaattctaga

SEQ ID NO: 89 - RNA, 20 nt, synthetic construct
  source 1..20: mol_type = other RNA
cgacttctgttttaggacag

SEQ ID NO: 90 - RNA, 20 nt, synthetic construct
  source 1..20: mol_type = other RNA
gacttctgttttaggacaga

SEQ ID NO: 91 - RNA, 20 nt, synthetic construct
  source 1..20: mol_type = other RNA
ggtcagcatgccaatatgtg

SEQ ID NO: 92 - RNA, 20 nt, synthetic construct
  source 1..20: mol_type = other RNA
gtcagcatgccaatatgtgt

SEQ ID NO: 93 - RNA, 20 nt, synthetic construct
  source 1..20: mol_type = other RNA
tcagcatgccaatatgtgtg

SEQ ID NO: 94 - RNA, 20 nt, synthetic construct
  source 1..20: mol_type = other RNA
cagcatgccaatatgtgtgg

SEQ ID NO: 95 - RNA, 20 nt, synthetic construct
  source 1..20: mol_type = other RNA
gccaatatgtgtgggggcta

SEQ ID NO: 96 - RNA, 20 nt, synthetic construct
  source 1..20: mol_type = other RNA
ggctacggccatgcagcgca

SEQ ID NO: 97 - RNA, 20 nt, synthetic construct
  source 1..20: mol_type = other RNA
cagcgcatggctcatgtgga

SEQ ID NO: 98 - RNA, 20 nt, synthetic construct
  source 1..20: mol_type = other RNA
cttcctcctacctctcacag

SEQ ID NO: 99 - RNA, 20 nt, synthetic construct
  source 1..20: mol_type = other RNA
ttcaattgaggaggtggccc

SEQ ID NO: 100 - RNA, 20 nt, synthetic construct
  source 1..20: mol_type = other RNA
ctcctcaattgaagaagtgg

SEQ ID NO: 101 - RNA, 20 nt, synthetic construct
  source 1..20: mol_type = other RNA
ttccgccacttcttcaattg

SEQ ID NO: 102 - RNA, 20 nt, synthetic construct
  source 1..20: mol_type = other RNA
attgaagaagtggcggaagc

SEQ ID NO: 103 - RNA, 20 nt, synthetic construct
  source 1..20: mol_type = other RNA
agtggcggaagctggtcctg

SEQ ID NO: 104 - RNA, 20 nt, synthetic construct
  source 1..20: mol_type = other RNA
tgcagccaacgaagtgcctc

SEQ ID NO: 105 - RNA, 20 nt, synthetic construct
  source 1..20: mol_type = other RNA
gctgcaactgtatatctaca

SEQ ID NO: 106 - RNA, 20 nt, synthetic construct
  source 1..20: mol_type = other RNA
ctagcttcttggtgacttct

SEQ ID NO: 107 - RNA, 20 nt, synthetic construct
  source 1..20: mol_type = other RNA
aagtcaccaagaagctagtg

SEQ ID NO: 108 - RNA, 20 nt, synthetic construct
  source 1..20: mol_type = other RNA
caccaagaagctagtgcggc

SEQ ID NO: 109 - RNA, 20 nt, synthetic construct
  source 1..20: mol_type = other RNA
tgcctgccgcactagcttct

SEQ ID NO: 110 - RNA, 20 nt, synthetic construct
  source 1..20: mol_type = other RNA
agtgcggcaggcagagaaga

SEQ ID NO: 111 - RNA, 20 nt, synthetic construct
  source 1..20: mol_type = other RNA
gtgcggcaggcagagaagat

SEQ ID NO: 112 - RNA, 20 nt, synthetic construct
  source 1..20: mol_type = other RNA
ggcagagaagatgggctaca

SEQ ID NO: 113 - RNA, 20 nt, synthetic construct
  source 1..20: mol_type = other RNA
accttacctgggcaaccgtc

SEQ ID NO: 114 - RNA, 20 nt, synthetic construct
  source 1..20: mol_type = other RNA
tccagacggttgcccaggta

SEQ ID NO: 115 - RNA, 20 nt, synthetic construct
  source 1..20: mol_type = other RNA
catcatccagacggttgccc

SEQ ID NO: 116 - RNA, 20 nt, synthetic construct
  source 1..20: mol_type = other RNA
tgttacgcacatcatccaga

SEQ ID NO: 117 - RNA, 20 nt, synthetic construct
  source 1..20: mol_type = other RNA
catggttacctgagttgtgg

SEQ ID NO: 118 - RNA, 20 nt, synthetic construct
  source 1..20: mol_type = other RNA
gatcatggttacctgagttg

SEQ ID NO: 119 - RNA, 20 nt, synthetic construct
  source 1..20: mol_type = other RNA
tcgtctccaaaattttcctc

SEQ ID NO: 120 - RNA, 20 nt, synthetic construct
  source 1..20: mol_type = other RNA
gaaaattttggagacgacag

SEQ ID NO: 121 - RNA, 20 nt, synthetic construct
  source 1..20: mol_type = other RNA
caatagacccatctatcagc

SEQ ID NO: 122 - RNA, 20 nt, synthetic construct
  source 1..20: mol_type = other RNA
tatcttcccagctgatagat

SEQ ID NO: 123 - RNA, 20 nt, synthetic construct
  source 1..20: mol_type = other RNA
atatcttcccagctgataga

SEQ ID NO: 124 - RNA, 20 nt, synthetic construct
  source 1..20: mol_type = other RNA
gcgtctgccaaaactcacag

SEQ ID NO: 125 - RNA, 20 nt, synthetic construct
  source 1..20: mol_type = other RNA
gccagaaattgtggaggctg

SEQ ID NO: 126 - RNA, 20 nt, synthetic construct
  source 1..20: mol_type = other RNA
tccacagcctccacaatttc

SEQ ID NO: 127 - RNA, 20 nt, synthetic construct
  source 1..20: mol_type = other RNA
gaaattgtggaggctgtgga

SEQ ID NO: 128 - RNA, 20 nt, synthetic construct
  source 1..20: mol_type = other RNA
aaattgtggaggctgtggaa

SEQ ID NO: 129 - RNA, 20 nt, synthetic construct
  source 1..20: mol_type = other RNA
tgtggaggctgtggaaggga

SEQ ID NO: 130 - RNA, 20 nt, synthetic construct
  source 1..20: mol_type = other RNA
ggaggctgtggaagggaagg

SEQ ID NO: 131 - RNA, 20 nt, synthetic construct
  source 1..20: mol_type = other RNA
ttgtggggagaccaatcgtt

SEQ ID NO: 132 - RNA, 20 nt, synthetic construct
  source 1..20: mol_type = other RNA
tgtggggagaccaatcgttt

SEQ ID NO: 133 - RNA, 20 nt, synthetic construct
  source 1..20: mol_type = other RNA
gtggggagaccaatcgtttg

SEQ ID NO: 134 - RNA, 20 nt, synthetic construct
  source 1..20: mol_type = other RNA
aaagctaagccccaaacgat

SEQ ID NO: 135 - RNA, 20 nt, synthetic construct
  source 1..20: mol_type = other RNA
catttctttgtccagttacc

SEQ ID NO: 136 - RNA, 20 nt, synthetic construct
  source 1..20: mol_type = other RNA
tgtatcttttcacttggtta

SEQ ID NO: 137 - RNA, 20 nt, synthetic construct
  source 1..20: mol_type = other RNA
gtatcttttcacttggttag

SEQ ID NO: 138 - RNA, 20 nt, synthetic construct
  source 1..20: mol_type = other RNA
tatcttttcacttggttagg

SEQ ID NO: 139 - RNA, 20 nt, synthetic construct
  source 1..20: mol_type = other RNA
agatgtcctcgagatactaa

SEQ ID NO: 140 - RNA, 20 nt, synthetic construct
  source 1..20: mol_type = other RNA
attcttcctttagtatctcg

SEQ ID NO: 141 - RNA, 20 nt, synthetic construct
  source 1..20: mol_type = other RNA
actaaaggaagaattccggt

SEQ ID NO: 142 - RNA, 20 nt, synthetic construct
  source 1..20: mol_type = other RNA
cactcagagccatggccaac

SEQ ID NO: 143 - RNA, 20 nt, synthetic construct
  source 1..20: mol_type = other RNA
agtcttaccactcagagcca

SEQ ID NO: 144 - RNA, 20 nt, synthetic construct
  source 1..20: mol_type = other RNA
atgtatgcattattttttca

SEQ ID NO: 145 - RNA, 20 nt, synthetic construct
  source 1..20: mol_type = other RNA
attggtgaggaaaaatcctt

SEQ ID NO: 146 - RNA, 20 nt, synthetic construct
  source 1..20: mol_type = other RNA
tattgtgcactgtcagatct

SEQ ID NO: 147 - RNA, 20 nt, synthetic construct
  source 1..20: mol_type = other RNA
gccagactccaagttctgcc

SEQ ID NO: 148 - RNA, 20 nt, synthetic construct
  source 1..20: mol_type = other RNA
taaggccagtggaaagaatt

SEQ ID NO: 149 - RNA, 20 nt, synthetic construct
  source 1..20: mol_type = other RNA
ggcagcgaggagtccacagt

SEQ ID NO: 150 - RNA, 20 nt, synthetic construct
  source 1..20: mol_type = other RNA
tctttccactggccttaacc

SEQ ID NO: 151 - RNA, 100 nt, synthetic construct
  source 1..100: mol_type = other RNA
aacctgtgaaaatgctccccgttttagagctagaaatagcaagttaaaataaggctagtccgttatcaacttgaaaaagtggcaccgagtcggtgctttt

SEQ ID NO: 152 - RNA, 100 nt, synthetic construct
  source 1..100: mol_type = other RNA
tgttctgccagaaattgtgggttttagagctagaaatagcaagttaaaataaggctagtccgttatcaacttgaaaaagtggcaccgagtcggtgctttt

SEQ ID NO: 153 - RNA, 100 nt, synthetic construct
  source 1..100: mol_type = other RNA
gcccgttcccagggactgacgttttagagctagaaatagcaagttaaaataaggctagtccgttatcaacttgaaaaagtggcaccgagtcggtgctttt

SEQ ID NO: 154 - RNA, 100 nt, synthetic construct
  source 1..100: mol_type = other RNA
aggagaaaatgataaagtacgttttagagctagaaatagcaagttaaaataaggctagtccgttatcaacttgaaaaagtggcaccgagtcggtgctttt

SEQ ID NO: 155 - RNA, 100 nt, synthetic construct
  source 1..100: mol_type = other RNA
aatagacccatctatcagctgttttagagctagaaatagcaagttaaaataaggctagtccgttatcaacttgaaaaagtggcaccgagtcggtgctttt

SEQ ID NO: 156 - RNA, 100 nt, synthetic construct
  source 1..100: mol_type = other RNA
atatatgactattacaggtcgttttagagctagaaatagcaagttaaaataaggctagtccgttatcaacttgaaaaagtggcaccgagtcggtgctttt

SEQ ID NO: 157 - RNA, 100 nt, synthetic construct
  source 1..100: mol_type = other RNA
ctcctcaattgaagaagtgggttttagagctagaaatagcaagttaaaataaggctagtccgttatcaacttgaaaaagtggcaccgagtcggtgctttt

SEQ ID NO: 158 - RNA, 100 nt, synthetic construct
  source 1..100: mol_type = other RNA
gctgcaactgtatatctacagttttagagctagaaatagcaagttaaaataaggctagtccgttatcaacttgaaaaagtggcaccgagtcggtgctttt

SEQ ID NO: 159 - RNA, 100 nt, synthetic construct
  source 1..100: mol_type = other RNA
accttacctgggcaaccgtcgttttagagctagaaatagcaagttaaaataaggctagtccgttatcaacttgaaaaagtggcaccgagtcggtgctttt

SEQ ID NO: 160 - RNA, 100 nt, synthetic construct
  source 1..100: mol_type = other RNA
attggtgaggaaaaatccttgttttagagctagaaatagcaagttaaaataaggctagtccgttatcaacttgaaaaagtggcaccgagtcggtgctttt

SEQ ID NO: 161 - RNA, 100 nt, synthetic construct; identical to SEQ ID NO: 151
  source 1..100: mol_type = other RNA

SEQ ID NO: 162 - RNA, 100 nt, synthetic construct; identical to SEQ ID NO: 152
  source 1..100: mol_type = other RNA

SEQ ID NO: 163 - RNA, 100 nt, synthetic construct; identical to SEQ ID NO: 153
  source 1..100: mol_type = other RNA

SEQ ID NO: 164 - RNA, 100 nt, synthetic construct; identical to SEQ ID NO: 155
  source 1..100: mol_type = other RNA

SEQ ID NO: 165 - RNA, 88 nt, synthetic construct
  source 1..88: mol_type = other RNA
aacctgtgaaaatgctccccgttttagagctagaaatagcaagttaaaataaggctagtccgttatcaacttggcaccgagtcggtgc

SEQ ID NO: 166 - RNA, 88 nt, synthetic construct
  source 1..88: mol_type = other RNA
tgttctgccagaaattgtgggttttagagctagaaatagcaagttaaaataaggctagtccgttatcaacttggcaccgagtcggtgc

SEQ ID NO: 167 - RNA, 88 nt, synthetic construct
  source 1..88: mol_type = other RNA
gcccgttcccagggactgacgttttagagctagaaatagcaagttaaaataaggctagtccgttatcaacttggcaccgagtcggtgc

SEQ ID NO: 168 - RNA, 88 nt, synthetic construct
  source 1..88: mol_type = other RNA
aatagacccatctatcagctgttttagagctagaaatagcaagttaaaataaggctagtccgttatcaacttggcaccgagtcggtgc

SEQ ID NO: 169 - RNA, 100 nt, synthetic construct
  source 1..100: mol_type = other RNA
tcactgatgcagaccagtcggttttagagctagaaatagcaagttaaaataaggctagtccgttatcaacttgaaaaagtggcaccgagtcggtgctttt

SEQ ID NO: 170 - RNA, 100 nt, synthetic construct
  source 1..100: mol_type = other RNA
cacgtgagccatgcactgcagttttagagctagaaatagcaagttaaaataaggctagtccgttatcaacttgaaaaagtggcaccgagtcggtgctttt

SEQ ID NO: 171 - RNA, 100 nt, synthetic construct
  source 1..100: mol_type = other RNA
tcttttcttacctcgcacaggttttagagctagaaatagcaagttaaaataaggctagtccgttatcaacttgaaaaagtggcaccgagtcggtgctttt

SEQ ID NO: 172-199 - skipped (28 entries)

SEQ ID NO: 200 - RNA, 22 nt, synthetic construct
  source 1..22: mol_type = other RNA
gttttagagctatgctgttttg

SEQ ID NO: 201 - RNA, 80 nt, synthetic construct
  source 1..80: mol_type = other RNA
gttttagagctagaaatagcaagttaaaataaggctagtccgttatcaacttgaaaaagtggcaccgagtcggtgctttt

SEQ ID NO: 202 - RNA, 68 nt, synthetic construct
  source 1..68: mol_type = other RNA
gttttagagctagaaatagcaagttaaaataaggctagtccgttatcaacttggcaccgagtcggtgc

SEQ ID NO: 203 - RNA, 76 nt, synthetic construct
  source 1..76: mol_type = other RNA
gttttagagctagaaatagcaagttaaaataaggctagtccgttatcaacttgaaaaagtggcaccgagtcggtgc

SEQ ID NO: 204-250 - skipped (47 entries)

SEQ ID NO: 251 - RNA, 100 nt, synthetic construct; identical to SEQ ID NO: 151
  source 1..100: mol_type = other RNA

SEQ ID NO: 252 - RNA, 101 nt, synthetic construct
  source 1..101: mol_type = other RNA
tgtctctgccagaaattgtgggttttagagctagaaatagcaagttaaaataaggctagtccgttatcaacttgaaaaagtggcaccgagtcggtgctttt

SEQ ID NO: 253 - RNA, 100 nt, synthetic construct; identical to SEQ ID NO: 153
  source 1..100: mol_type = other RNA

SEQ ID NO: 254 - RNA, 100 nt, synthetic construct; identical to SEQ ID NO: 154
  source 1..100: mol_type = other RNA

SEQ ID NO: 255 - RNA, 100 nt, synthetic construct; identical to SEQ ID NO: 155
  source 1..100: mol_type = other RNA

SEQ ID NO: 256 - RNA, 100 nt, synthetic construct; identical to SEQ ID NO: 156
  source 1..100: mol_type = other RNA

SEQ ID NO: 257 - RNA, 100 nt, synthetic construct; identical to SEQ ID NO: 157
  source 1..100: mol_type = other RNA

SEQ ID NO: 258 - RNA, 100 nt, synthetic construct; identical to SEQ ID NO: 158
  source 1..100: mol_type = other RNA

SEQ ID NO: 259 - RNA, 100 nt, synthetic construct; identical to SEQ ID NO: 159
  source 1..100: mol_type = other RNA

SEQ ID NO: 260 - RNA, 100 nt, synthetic construct; identical to SEQ ID NO: 160
  source 1..100: mol_type = other RNA

SEQ ID NO: 261 - RNA, 100 nt, synthetic construct; identical to SEQ ID NO: 151
  source 1..100: mol_type = other RNA

SEQ ID NO: 262 - RNA, 100 nt, synthetic construct; identical to SEQ ID NO: 152
  source 1..100: mol_type = other RNA

SEQ ID NO: 263 - RNA, 100 nt, synthetic construct; identical to SEQ ID NO: 153
  source 1..100: mol_type = other RNA

SEQ ID NO: 264 - RNA, 100 nt, synthetic construct; identical to SEQ ID NO: 155
  source 1..100: mol_type = other RNA

SEQ ID NO: 265 - RNA, 88 nt, synthetic construct; identical to SEQ ID NO: 165
  source 1..88: mol_type = other RNA

SEQ ID NO: 266 - RNA, 88 nt, synthetic construct; identical to SEQ ID NO: 166
  source 1..88: mol_type = other RNA

SEQ ID NO: 267 - RNA, 88 nt, synthetic construct; identical to SEQ ID NO: 167
  source 1..88: mol_type = other RNA

SEQ ID NO: 268 - RNA, 88 nt, synthetic construct; identical to SEQ ID NO: 168
  source 1..88: mol_type = other RNA

SEQ ID NO: 269-299 - skipped (31 entries)

SEQ ID NO: 300 - RNA, 100 nt, synthetic construct
  source 1..100: mol_type = other RNA
nnnnnnnnnnnnnnnnnnnngttttagagctagaaatagcaagttaaaataaggctagtccgttatcaacttgaaaaagtggcaccgagtcggtgctttt

SEQ ID NO: 301-399 - skipped (99 entries)

SEQ ID NO: 400 - RNA, 80 nt, synthetic construct; identical to SEQ ID NO: 201
  source 1..80: mol_type = other RNA

SEQ ID NO: 401 - RNA, 80 nt, synthetic construct; identical to SEQ ID NO: 201
  source 1..80: mol_type = other RNA

SEQ ID NO: 402 - RNA, 80 nt, synthetic construct; identical to SEQ ID NO: 201
  source 1..80: mol_type = other RNA

SEQ ID NO: 403 - RNA, 80 nt, synthetic construct; identical to SEQ ID NO: 201
  source 1..80: mol_type = other RNA

SEQ ID NO: 404 - RNA, 80 nt, synthetic construct; identical to SEQ ID NO: 201
  source 1..80: mol_type = other RNA

SEQ ID NO: 405 - RNA, 80 nt, synthetic construct; identical to SEQ ID NO: 201
  source 1..80: mol_type = other RNA

SEQ ID NO: 406 - RNA, 80 nt, synthetic construct; identical to SEQ ID NO: 201
  source 1..80: mol_type = other RNA

SEQ ID NO: 407 - RNA, 80 nt, synthetic construct; identical to SEQ ID NO: 201
  source 1..80: mol_type = other RNA

SEQ ID NO: 408 - RNA, 80 nt, synthetic construct; identical to SEQ ID NO: 201
  source 1..80: mol_type = other RNA

SEQ ID NO: 409 - RNA, 80 nt, synthetic construct; identical to SEQ ID NO: 201
  source 1..80: mol_type = other RNA

SEQ ID NO: 410 - RNA, 80 nt, synthetic construct; identical to SEQ ID NO: 201
  source 1..80: mol_type = other RNA

SEQ ID NO: 411 - RNA, 80 nt, synthetic construct; identical to SEQ ID NO: 201
  source 1..80: mol_type = other RNA

SEQ ID NO: 412 - RNA, 80 nt, synthetic construct; identical to SEQ ID NO: 201
  source 1..80: mol_type = other RNA

SEQ ID NO: 413 - RNA, 80 nt, synthetic construct; identical to SEQ ID NO: 201
  source 1..80: mol_type = other RNA

SEQ ID NO: 414 - RNA, 80 nt, synthetic construct; identical to SEQ ID NO: 201
  source 1..80: mol_type = other RNA

SEQ ID NO: 415 - RNA, 80 nt, synthetic construct; identical to SEQ ID NO: 201
  source 1..80: mol_type = other RNA

SEQ ID NO: 416 - RNA, 80 nt, synthetic construct; identical to SEQ ID NO: 201
  source 1..80: mol_type = other RNA

SEQ ID NO: 417 - RNA, 80 nt, synthetic construct; identical to SEQ ID NO: 201
  source 1..80: mol_type = other RNA

SEQ ID NO: 418 - RNA, 80 nt, synthetic construct; identical to SEQ ID NO: 201
  source 1..80: mol_type = other RNA

SEQ ID NO: 419 - RNA, 80 nt, synthetic construct; identical to SEQ ID NO: 201
  source 1..80: mol_type = other RNA

SEQ ID NO: 420 - RNA, 80 nt, synthetic construct; identical to SEQ ID NO: 201
  source 1..80: mol_type = other RNA

SEQ ID NO: 421 - RNA, 80 nt, synthetic construct; identical to SEQ ID NO: 201
  source 1..80: mol_type = other RNA

SEQ ID NO: 422 - RNA, 80 nt, synthetic construct; identical to SEQ ID NO: 201
  source 1..80: mol_type = other RNA

SEQ ID NO: 423 - RNA, 80 nt, synthetic construct; identical to SEQ ID NO: 201
  source 1..80: mol_type = other RNA

SEQ ID NO: 424 - RNA, 80 nt, synthetic construct; identical to SEQ ID NO: 201
  source 1..80: mol_type = other RNA

SEQ ID NO: 425 - RNA, 80 nt, synthetic construct; identical to SEQ ID NO: 201
  source 1..80: mol_type = other RNA

SEQ ID NO: 426 - RNA, 80 nt, synthetic construct; identical to SEQ ID NO: 201
  source 1..80: mol_type = other RNA

SEQ ID NO: 427 - RNA, 80 nt, synthetic construct; identical to SEQ ID NO: 201
  source 1..80: mol_type = other RNA

SEQ ID NO: 428 - RNA, 80 nt, synthetic construct; identical to SEQ ID NO: 201
  source 1..80: mol_type = other RNA

SEQ ID NO: 429 - RNA, 80 nt, synthetic construct; identical to SEQ ID NO: 201
  source 1..80: mol_type = other RNA

SEQ ID NO: 430 - RNA, 80 nt, synthetic construct; identical to SEQ ID NO: 201
  source 1..80: mol_type = other RNA

SEQ ID NO: 431 - RNA, 80 nt, synthetic construct; identical to SEQ ID NO: 201
  source 1..80: mol_type = other RNA

SEQ ID NO: 432 - RNA, 80 nt, synthetic construct; identical to SEQ ID NO: 201
  source 1..80: mol_type = other RNA

SEQ ID NO: 433 - RNA, 80 nt, synthetic construct; identical to SEQ ID NO: 201
  source 1..80: mol_type = other RNA

SEQ ID NO: 434 - RNA, 80 nt, synthetic construct; identical to SEQ ID NO: 201
  source 1..80: mol_type = other RNA

SEQ ID NO: 435 - RNA, 80 nt, synthetic construct; identical to SEQ ID NO: 201
  source 1..80: mol_type = other RNA

SEQ ID NO: 436 - RNA, 80 nt, synthetic construct; identical to SEQ ID NO: 201
  source 1..80: mol_type = other RNA

SEQ ID NO: 437 - RNA, 80 nt, synthetic construct; identical to SEQ ID NO: 201
  source 1..80: mol_type = other RNA

SEQ ID NO: 438 - RNA, 80 nt, synthetic construct; identical to SEQ ID NO: 201
  source 1..80: mol_type = other RNA

SEQ ID NO: 439 - RNA, 80 nt, synthetic construct; identical to SEQ ID NO: 201
  source 1..80: mol_type = other RNA

SEQ ID NO: 440 - RNA, 80 nt, synthetic construct; identical to SEQ ID NO: 201
  source 1..80: mol_type = other RNA

SEQ ID NO: 441 - RNA, 80 nt, synthetic construct; identical to SEQ ID NO: 201
  source 1..80: mol_type = other RNA

SEQ ID NO: 442 - RNA, 80 nt, synthetic construct; identical to SEQ ID NO: 201
  source 1..80: mol_type = other RNA

SEQ ID NO: 443 - RNA, 80 nt, synthetic construct; identical to SEQ ID NO: 201
  source 1..80: mol_type = other RNA

SEQ ID NO: 444 - RNA, 80 nt, synthetic construct; identical to SEQ ID NO: 201
  source 1..80: mol_type = other RNA

SEQ ID NO: 445 - RNA, 80 nt, synthetic construct; identical to SEQ ID NO: 201
  source 1..80: mol_type = other RNA

SEQ ID NO: 446 - RNA, 80 nt, synthetic construct; identical to SEQ ID NO: 201
  source 1..80: mol_type = other RNA

SEQ ID NO: 447 - RNA, 80 nt, synthetic construct; identical to SEQ ID NO: 201
  source 1..80: mol_type = other RNA

SEQ ID NO: 448-449 - skipped (2 entries)

SEQ ID NO: 450 - RNA, 68 nt, synthetic construct; identical to SEQ ID NO: 202
  source 1..68: mol_type = other RNA

SEQ ID NO: 451-499 - skipped (49 entries)

SEQ ID NO: 500 - DNA, 4104 bp, synthetic construct
  source 1..4104: mol_type = other DNA
atggataagaagtactcaatcgggctggatatcggaactaattccgtgggttgggcagtgatcacggatgaatacaaagtgccgtccaagaagttcaaggtcctggggaacaccgatagacacagcatcaagaaaaatctcatcggagccctgctgtttgactccggcgaaaccgcagaagcgacccggctcaaacgtaccgcgaggcgacgctacacccggcggaagaatcgcatctgctatctgcaagagatcttttcgaacgaaatggcaaaggtcgacgacagcttcttccaccgcctggaagaatctttcctggtggaggaggacaagaagcatgaacggcatcctatctttggaaacatcgtcgacgaagtggcgtaccacgaaaagtacccgaccatctaccatctgcggaagaagttggttgactcaactgacaaggccgacctcagattgatctacttggccctcgcccatatgatcaaattccgcggacacttcctgatcgaaggcgatctgaaccctgataactccgacgtggataagcttttcattcaactggtgcagacctacaaccaactgttcgaagaaaacccaatcaatgctagcggcgtcgatgccaaggccatcctgtccgcccggctgtcgaagtcgcggcgcctcgaaaacctgatcgcacagctgccgggagagaaaaagaacggacttttcggcaacttgatcgctctctcactgggactcactcccaatttcaagtccaattttgacctggccgaggacgcgaagctgcaactctcaaaggacacctacgacgacgacttggacaatttgctggcacaaattggcgatcagtacgcggatctgttccttgccgctaagaacctttcggacgcaatcttgctgtccgatatcctgcgcgtgaacaccgaaataaccaaagcgccgcttagcgcctcgatgattaagcggtacgacgagcatcaccaggatctcacgctgctcaaagcgctcgtgagacagcaactgcctgaaaagtacaaggagatcttcttcgaccagtccaagaatgggtacgcagggtacatcgatggaggcgctagccaggaagagttctataagttcatcaagccaatcctggaaaagatggacggaaccgaagaactgctggtcaagctgaacagggaggatctgctccggaaacagagaacctttgacaacggatccattccccaccagatccatctgggtgagctgcacgccatcttgcggcgccaggaggacttttacccattcctcaaggacaaccgggaaaagatcgagaaaattctgacgttccgcatcccgtattacgtgggcccactggcgcgcggcaattcgcgcttcgcgtggatgactagaaaatcagaggaaaccatcactccttggaatttcgaggaagttgtggataagggagcttcggcacaaagcttcatcgaacgaatgaccaacttcgacaagaatctcccaaacgagaaggtgcttcctaagcacagcctcctttacgaatacttcactgtctacaacgaactgactaaagtgaaatacgttactgaaggaatgaggaagccggcctttctgtccggagaacagaagaaagcaattgtcgatctgctgttcaagaccaaccgcaaggtgaccgtcaagcagcttaaagaggactacttcaagaagatcgagtgtttcgactcagtggaaatcagcggggtggaggacagattcaacgcttcgctgggaacctatcatgatctcctgaagatcatcaaggacaaggacttccttgacaacgaggagaacgaggacatcctggaagatatcgtcctgaccttgacccttttcgaggatcgcgagatgatcgaggagaggcttaagacctacgctcatctcttcgacgataaggtcatgaaacaactcaagcgccgccggtacactggttggggccgcctctcccgcaagctgatcaacggtattcgcgataaacagagcggtaaaactatcctggatttcctcaaatcggatggcttcgctaatcgtaacttcatgcaattgatccacgacgacagcctgacctttaaggaggacatccaaaaagcacaagtgtccggacagggagactcactccatgaacacatcgcgaatctggccggttcgccggcgattaagaagggaattctgcaaactgtgaaggtggtcgacgagctggtgaaggtcatgggacggcacaaaccggagaatatcgtgattgaaatggcccgagaaaaccagactacccagaagggccagaaaaactcccgcgaaaggatgaagcggatcgaagaaggaatcaaggagctgggcagccagatcctgaaagagcacccggtggaaaacacgcagctgcagaacgagaagctctacctgtactatttgcaaaatggacgggacatgtacgtggaccaagagctggacatcaatcggttgtctgattacgacgtggaccacatcgttccacagtcctttctgaaggatgactcgatcgataacaaggtgttgactcgcagcgacaagaacagagggaagtcagataatgtgccatcggaggaggtcgtgaagaagatgaagaattactggcggcagctcctgaatgcgaagctgattacccagagaaagtttgacaatctcactaaagccgagcgcggcggactctcagagctggataaggctggattcatcaaacggcagctggtcgagactcggcagattaccaagcacgtggcgcagatcttggactcccgcatgaacactaaatacgacgagaacgataagctcatccgggaagtgaaggtgattaccctgaaaagcaaacttgtgtcggactttcggaaggactttcagttttacaaagtgagagaaatcaacaactaccatcacgcgcatgacgcatacctcaacgctgtggtcggtaccgccctgatcaaaaagtaccctaaacttgaatcggagtttgtgtacggagactacaaggtctacgacgtgaggaagatgatagccaagtccgaacaggaaatcgggaaagcaactgcgaaatacttcttttactcaaacatcatgaactttttcaagactgaaattacgctggccaatggagaaatcaggaagaggccactgatcgaaactaacggagaaacgggcgaaatcgtgtgggacaagggcagggacttcgcaactgttcgcaaagtgctctctatgccgcaagtcaatattgtgaagaaaaccgaagtgcaaaccggcggattttcaaaggaatcgatcctcccaaagagaaatagcgacaagctcattgcacgcaagaaagactgggacccgaagaagtacggaggattcgattcgccgactgtcgcatactccgtcctcgtggtggccaaggtggagaagggaaagagcaaaaagctcaaatccgtcaaagagctgctggggattaccatcatggaacgatcctcgttcgagaagaacccgattgatttcctcgaggcgaagggttacaaggaggtgaagaaggatctgatcatcaaactccccaagtactcactgttcgaactggaaaatggtcggaagcgcatgctggcttcggccggagaactccaaaaaggaaatgagctggccttgcctagcaagtacgtcaacttcctctatcttgcttcgcactacgaaaaactcaaagggtcaccggaagataacgaacagaagcagcttttcgtggagcagcacaagcattatctggatgaaatcatcgaacaaatctccgagttttcaaagcgcgtgatcctcgccgacgccaacctcgacaaagtcctgtcggcctacaataagcatagagataagccgatcagagaacaggccgagaacattatccacttgttcaccctgactaacctgggagccccagccgccttcaagtacttcgatactactatcgatcgcaaaagatacacgtccaccaaggaagttctggacgcgaccctgatccaccaaagcatcactggactctacgaaactaggatcgatctgtcgcagctgggtggcgat

SEQ ID NO: 501 - DNA, 4411 bp, synthetic construct
  source 1..4411: mol_type = other DNA
gggtcccgcagtcggcgtccagcggctctgcttgttcgtgtgtgtgtcgttgcaggccttattcggatccgccaccatggacaagaagtacagcatcggactggacatcggaacaaacagcgtcggatgggcagtcatcacagacgaatacaaggtcccgagcaagaagttcaaggtcctgggaaacacagacagacacagcatcaagaagaacctgatcggagcactgctgttcgacagcggagaaacagcagaagcaacaagactgaagagaacagcaagaagaagatacacaagaagaaagaacagaatctgctacctgcaggaaatcttcagcaacgaaatggcaaaggtcgacgacagcttcttccacagactggaagaaagcttcctggtcgaagaagacaagaagcacgaaagacacccgatcttcggaaacatcgtcgacgaagtcgcataccacgaaaagtacccgacaatctaccacctgagaaagaagctggtcgacagcacagacaaggcagacctgagactgatctacctggcactggcacacatgatcaagttcagaggacacttcctgatcgaaggagacctgaacccggacaacagcgacgtcgacaagctgttcatccagctggtccagacatacaaccagctgttcgaagaaaacccgatcaacgcaagcggagtcgacgcaaaggcaatcctgagcgcaagactgagcaagagcagaagactggaaaacctgatcgcacagctgccgggagaaaagaagaacggactgttcggaaacctgatcgcactgagcctgggactgacaccgaacttcaagagcaacttcgacctggcagaagacgcaaagctgcagctgagcaaggacacatacgacgacgacctggacaacctgctggcacagatcggagaccagtacgcagacctgttcctggcagcaaagaacctgagcgacgcaatcctgctgagcgacatcctgagagtcaacacagaaatcacaaaggcaccgctgagcgcaagcatgatcaagagatacgacgaacaccaccaggacctgacactgctgaaggcactggtcagacagcagctgccggaaaagtacaaggaaatcttcttcgaccagagcaagaacggatacgcaggatacatcgacggaggagcaagccaggaagaattctacaagttcatcaagccgatcctggaaaagatggacggaacagaagaactgctggtcaagctgaacagagaagacctgctgagaaagcagagaacattcgacaacggaagcatcccgcaccagatccacctgggagaactgcacgcaatcctgagaagacaggaagacttctacccgttcctgaaggacaacagagaaaagatcgaaaagatcctgacattcagaatcccgtactacgtcggaccgctggcaagaggaaacagcagattcgcatggatgacaagaaagagcgaagaaacaatcacaccgtggaacttcgaagaagtcgtcgacaagggagcaagcgcacagagcttcatcgaaagaatgacaaacttcgacaagaacctgccgaacgaaaaggtcctgccgaagcacagcctgctgtacgaatacttcacagtctacaacgaactgacaaaggtcaagtacgtcacagaaggaatgagaaagccggcattcctgagcggagaacagaagaaggcaatcgtcgacctgctgttcaagacaaacagaaaggtcacagtcaagcagctgaaggaagactacttcaagaagatcgaatgcttcgacagcgtcgaaatcagcggagtcgaagacagattcaacgcaagcctgggaacataccacgacctgctgaagatcatcaaggacaaggacttcctggacaacgaagaaaacgaagacatcctggaagacatcgtcctgacactgacactgttcgaagacagagaaatgatcgaagaaagactgaagacatacgcacacctgttcgacgacaaggtcatgaagcagctgaagagaagaagatacacaggatggggaagactgagcagaaagctgatcaacggaatcagagacaagcagagcggaaagacaatcctggacttcctgaagagcgacggattcgcaaacagaaacttcatgcagctgatccacgacgacagcctgacattcaaggaagacatccagaaggcacaggtcagcggacagggagacagcctgcacgaacacatcgcaaacctggcaggaagcccggcaatcaagaagggaatcctgcagacagtcaaggtcgtcgacgaactggtcaaggtcatgggaagacacaagccggaaaacatcgtcatcgaaatggcaagagaaaaccagacaacacagaagggacagaagaacagcagagaaagaatgaagagaatcgaagaaggaatcaaggaactgggaagccagatcctgaaggaacacccggtcgaaaacacacagctgcagaacgaaaagctgtacctgtactacctgcagaacggaagagacatgtacgtcgaccaggaactggacatcaacagactgagcgactacgacgtcgaccacatcgtcccgcagagcttcctgaaggacgacagcatcgacaacaaggtcctgacaagaagcgacaagaacagaggaaagagcgacaacgtcccgagcgaagaagtcgtcaagaagatgaagaactactggagacagctgctgaacgcaaagctgatcacacagagaaagttcgacaacctgacaaaggcagagagaggaggactgagcgaactggacaaggcaggattcatcaagagacagctggtcgaaacaagacagatcacaaagcacgtcgcacagatcctggacagcagaatgaacacaaagtacgacgaaaacgacaagctgatcagagaagtcaaggtcatcacactgaagagcaagctggtcagcgacttcagaaaggacttccagttctacaaggtcagagaaatcaacaactaccaccacgcacacgacgcatacctgaacgcagtcgtcggaacagcactgatcaagaagtacccgaagctggaaagcgaattcgtctacggagactacaaggtctacgacgtcagaaagatgatcgcaaagagcgaacaggaaatcggaaaggcaacagcaaagtacttcttctacagcaacatcatgaacttcttcaagacagaaatcacactggcaaacggagaaatcagaaagagaccgctgatcgaaacaaacggagaaacaggagaaatcgtctgggacaagggaagagacttcgcaacagtcagaaaggtcctgagcatgccgcaggtcaacatcgtcaagaagacagaagtccagacaggaggattcagcaaggaaagcatcctgccgaagagaaacagcgacaagctgatcgcaagaaagaaggactgggacccgaagaagtacggaggattcgacagcccgacagtcgcatacagcgtcctggtcgtcgcaaaggtcgaaaagggaaagagcaagaagctgaagagcgtcaaggaactgctgggaatcacaatcatggaaagaagcagcttcgaaaagaacccgatcgacttcctggaagcaaagggatacaaggaagtcaagaaggacctgatcatcaagctgccgaagtacagcctgttcgaactggaaaacggaagaaagagaatgctggcaagcgcaggagaactgcagaagggaaacgaactggcactgccgagcaagtacgtcaacttcctgtacctggcaagccactacgaaaagctgaagggaagcccggaagacaacgaacagaagcagctgttcgtcgaacagcacaagcactacctggacgaaatcatcgaacagatcagcgaattcagcaagagagtcatcctggcagacgcaaacctggacaaggtcctgagcgcatacaacaagcacagagacaagccgatcagagaacaggcagaaaacatcatccacctgttcacactgacaaacctgggagcaccggcagcattcaagtacttcgacacaacaatcgacagaaagagatacacaagcacaaaggaagtcctggacgcaacactgatccaccagagcatcacaggactgtacgaaacaagaatcgacctgagccagctgggaggagacggaggaggaagcccgaagaagaagagaaaggtctagctagccatcacatttaaaagcatctcagcctaccatgagaataagagaaagaaaatgaagatcaatagcttattcatctctttttctttttcgttggtgtaaagccaacaccctgtctaaaaaacataaatttctttaatcattttgcctcttttctctgtgcttcaattaataaaaaatggaaagaacctcgag

SEQ ID NO: 502-599 - skipped (98 entries)

SEQ ID NO: 600 - protein, 7 aa, synthetic construct
PKKKRKV

SEQ ID NO: 601 - protein, 7 aa, synthetic construct
PKKKRRV

SEQ ID NO: 602 - protein, 16 aa, synthetic construct
KRPAATKKAGQAKKKK